(19) United States
(12) Patent Application Publication
(10) Pub. No.: US 2025/0360220 A1
(43) Pub. Date: Nov. 27, 2025

(54) REGULATION OF TAU USING PROTEIN-LIKE POLYMERS AND USES THEREOF

(71) Applicant: NORTHWESTERN UNIVERSITY, Evanston, IL (US)

(72) Inventors: Nathan C. GIANNESCHI, Evanston, IL (US); Mara A. FATTAH, Evanston, IL (US); Richard I. MORIMOTO, Evanston, IL (US)

(73) Assignee: NORTHWESTERN UNIVERSITY, Evanston, IL (US)

(21) Appl. No.: 18/992,900

(22) PCT Filed: Jul. 13, 2023
(86) PCT No.: PCT/US2023/070163

§ 371 Date: Jan. 9, 2025

Related U.S. Application Data
(60) Provisional application No. 63/389,616, filed on Jul. 15, 2022.

(51) Int. Cl. A61K 47/58; A61K 47/64

(52) U.S. Cl. CPC A61K 47/58; A61K 47/64

(57) ABSTRACT

Disclosed are protein-like polymers and uses thereof. The protein-like polymers generally comprise a polymer of formula (FX1). The polymer of formula (FX1) in some aspects comprises a peptide that (i) inhibits aggregation of, (ii) accelerates aggregation of, (iii) binds to, and/or (iv) mimics: (a) at least a portion of Tau protein, and/or (b) at least a portion of microtubulin protein.

### CROSS-REFERENCE TO RELATED APPLICATIONS

This application claims the benefit of priority to U.S. Provisional Patent Application No. 63/389,616, filed Jul. 15, 2022, which is hereby incorporated by reference in its entirety.

### STATEMENT REGARDING FEDERALLY SPONSORED RESEARCH OR DEVELOPMENT

This invention was made with government support under Award Number 1F31AG076334-01, awarded by the Department of Health and Human Services, National Institute of Health. The government has certain rights in the invention.

### REFERENCE TO AN ELECTRONIC SEQUENCE LISTING

The content of the electronic sequence listing (339677_93-21_WO_ST1.xml; Size: 14,746 bytes; and Date of Creation: Jul. 11, 2023) is herein incorporated by reference in its entirety.

### BACKGROUND

Many hundreds of human diseases, collectively known as protein conformational diseases or protein folding disorders, result from protein misfolding due to intrinsic and extrinsic errors amplified by exposures to environmental and physiological stress conditions. Zhao J-H et al., Chemical Chaperone and Inhibitor Discovery: Potential Treatments for Protein Conformational Diseases. Perspect Medicin Chem 2007; 1:PMC.S212; Voisine C et al., Chaperone networks: Tipping the balance in protein folding diseases. Neurobiol Dis 2010; 40:12-20; Ciryam P et al., Widespread Aggregation and Neurodegenerative Diseases Are Associated with Supersaturated Proteins. Cell Rep 2013; 5:781-90. Such events challenge the integrity of the proteome and can lead to premature clearance, mislocalization, and dysfunction or aggregation of proteins, thus affecting cellular robustness, health, and longevity. These diseases include Type II diabetes, cystic fibrosis, cancer, and neurodegenerative diseases, as exemplified by Alzheimer's disease (AD), frontotemporal dementia, Parkinson's disease, amyotrophic lateral sclerosis (ALS), and Huntington's disease.

In each case, inhibiting, controlling and even probing the progression of these processes has proven elusive via traditional approaches including small molecule and antibody type systems. As described herein, we focus on neurodegenerative diseases, and specifically on the dysregulation and the off-pathway behavior of the microtubule-associated protein tau (MAPT) which is causative for tauopathies including AD, frontotemporal dementia, and traumatic brain injury. It is thought that mediating the dysregulation of tau should alleviate the pathogenesis of dementia in tauopathies. Here, we disclose an approach to probe and drug tau in transient, early, toxic states of amyloid formation by engaging the cellular machinery and phase-separated states of tau (FIG. 1). It is believed this approach may provide a foundation leading to the treatment of the larger class of age-associated degenerative diseases of protein misfolding.

We provide the use of proteomimetic material termed protein-like polymers (PLPs) to engage proteins and the quality control machinery within cells (FIG. 1). Callmann C E et al., Poly(peptide): Synthesis, Structure, and Function of Peptide-Polymer Amphiphiles and Protein-like Polymers. Acc Chem Res 2020; 53:400-13; Gianneschi N C et al., Biomolecular Densely Grafted Brush Polymers: Oligonucleotides, Oligosaccharides and Oligopeptides. Angew Chemie Int Ed 2020; Blum A P, Kammeyer J K, Gianneschi N C. Activating peptides for cellular uptake via polymerization into high density brushes. Chem Sci 2016; 7:989-94; Sun H et al., Peptide-Brush Polymers as Globular Proteomimetics. The proposed PLPs are modular, scalable, and rapidly formulated using advanced polymerization strategies, offering cell penetration, specific target binding, and multivalency. We believe that successfully integrating PLPs into native protein assemblies will have broad impacts in understanding and treating many diseases linked to misfolding of key proteins like TDP43, Huntingtin, and SOD1.

As a proof-of-concept, disclosed herein is a focus on a single protein/disease (tau/Alzheimer's), to create a robust methodological framework for translating these proteomimetic polymers into a therapeutic platform that can ultimately be widely adopted for protein misfolding disorders. It is believed that PLPs can be optimized to alter the biological consequences of the phase transition of tau protein into amyloid fibers. This strategy builds upon the evidence that misfolding and aggregation is the fundamental problem for tauopathies in Alzheimer's disease, yet current efforts to develop small molecule or antibody treatments have proven challenging at best and at worst have been unsuccessful.

Thus, there remains a need for therapeutics and methods targeting age-associated degenerative diseases of protein misfolding. The invention provides such therapeutics and methods. This and other advantages of the present invention will become apparent from the detailed description provided herein.

### SUMMARY OF THE INVENTION

In an aspect, the invention provides a polymer comprising a first repeating unit comprising a first polymer backbone subunit directly or indirectly covalently linked to a first functional sidechain comprising a peptide, which (i) inhibits aggregation of, (ii) accelerates aggregation of, (iii) binds to, and/or (iv) mimics at least a portion of tau protein.

The present invention further includes a polymer comprising a first repeating unit comprising a first polymer backbone subunit directly or indirectly covalently linked to a first functional sidechain comprising a peptide, which (i) inhibits aggregation of, (ii) accelerates aggregation of, (iii) binds to, and/or (iv) mimics at least a portion of microtubulin protein.

Further disclosed herein is a polymer characterized by a formula (FX1):

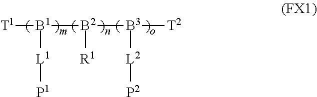

wherein: each P¹ independently comprises a peptide; each P² independently comprises a peptide, and each instance of P² is different from each instance of P¹; at least one P¹ independently, or in combination with other instances of P¹, (i) inhibits aggregation of, (ii) accelerates aggregation of, (iii) binds to, and/or (iv) mimics: (a) at least a portion of Tau protein, and/or (b) at least a portion of microtubulin protein; T¹ and T² are each independently polymer backbone terminating groups that can be the same or different; B¹, B², and B³ are each independently a polymer backbone subunit; L¹ and L² are each independently a linking group; R¹ is independently a substituent; m is an integer from 2 to 1000; n is an integer from 0 to 1000; o is an integer from 0 to 1000; each connecting line in the formula (FX1) represents a covalent linkage comprising at least one of a single bond, a double bond, one or more atoms, or any combination thereof, optionally wherein the one or more atoms comprise carbon, nitrogen, and/or oxygen atoms; each instance of B¹, B², B³, L¹, L², R¹, P¹, and P² is the same as or different from any other instance of B¹, B², B³, L¹, L², R¹, P¹, and P², respectively; and when (i) n is an integer from 1 to 1000, o is an integer from 1 to 1000, at least one instance of P¹ is different from another instance of P¹, and/or at least one instance of P² is different from another instance of P², then (ii) the polymer is a block copolymer or a statistical copolymer.

In other aspects, the present invention provides a method for preventing, disrupting, accelerating, or detecting Tau and/or microtubulin protein aggregation, the method comprising: contacting an oligomer, protofibril, amyloid fibril, and/or cross-β sheet amyloid species of Tau and/or microtubulin with a therapeutically effective amount of any of the polymers disclosed herein, or a composition thereof.

The present invention further includes a method for preventing, treating, or detecting a tauopathy-related disease or condition in a subject, the method comprising: administering to the subject a therapeutically effective amount of any of the polymers disclosed herein, or a composition thereof.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1: Polymer science-based proteomimetic approach to engaging tau and altering, detecting, tuning, and redirecting off-pathway aggregation. The on-pathway process (top left box) by which tau associates with microtubules in healthy neurons is disrupted, leading to off-pathway aggregates and amyloid (top right box). Protein-like polymers (PLPs) are designed to accelerate aggregation, inhibit aggregation, detect tau aggregates via a fluorescent label and initiate tau degradation by recruiting the ubiquitin-proteasome system. It is believed that PLPs act as decoys, capable of engaging phase separated states, altering the intercellular fate of tau.

FIG. 2: Engaged pathways in liquid-liquid phase separated state in cells. It is hypothesized that tau aggregates interact in these multiple novel pathways with embodiments of the PLPs that phase separate into tau condensates.

FIG. 3: Initial STEM results of PLP homopolymers incorporating either SEQ ID: 4 (top) or SEQ ID: 2 (bottom) as peptide sidechains.

FIG. 4: Bradford concentration assay of K18. K18-1 and K18-2 represent results from two separate runs of the same experimental protocol. K18-1: 1.657 μg/μl; 6.036 μl=10 μg. K18-2: 1.286 μg/μl; 7.780 μl=10 μg.

FIG. 5: Silver stain for purity of K18. Uncut K18 refers to His-tagged K18. TEV refers to TEV protease which was used to cleave His from K18. FPLC refers to fast protein liquid chromatography.

FIG. 6: Bradford concentration assay of K18 post dialysis into PBS with MgCl₂ and DTT. K18: 1.192 μg/μl; 6.036 μl=10 μg—Total volume: 24 ml.

FIG. 7A-7C: Polymerization confirmation results (FIG. 7A: molecular weight; FIG. 7C: polymerization kinetics) for a PLP homopolymer incorporating SEQ ID: 16 as peptide sidechains. Cell permeability assay results shown in FIG. 7B—scale: 10 μm.

FIG. 8: Polymerization kinetics for a PLP homopolymer incorporating SEQ ID: 1 (“TP3”) as peptide side chains.

FIG. 9A-9B: Polymerization confirmation results (FIG. 9A: molecular weight; FIG. 9B: polymerization kinetics) for a PLP homopolymer incorporating either SEQ ID: 4 (“TP4”) or SEQ ID: 2 (“TP7”) as peptide sidechains.

FIG. 10A-10B: Polymerization confirmation results (FIG. 10A: molecular weight; FIG. 10B: polymerization kinetics) for a PLP homopolymer incorporating either SEQ ID: 4 (“TP4”) or SEQ ID: 2 (“TP7”) as peptide sidechains.

FIG. 11A: Results from liquid-liquid phase separation (LLPS) protocols for a PLP homopolymer incorporating SEQ ID: 4 as peptide sidechains. HEPES: 10 mM HEPES buffer (pH 7.4) with 0.1M EDTA and 2 mM DTT. Media: Dulbecco Modified Eagles Medium (high glucose) with 2 mM L-glutamine with 10% Fetal Bovine Serum, 1% Penicillin/Streptomycin. Scale: 50 μm.

FIG. 11B: Results from liquid-liquid phase separation (LLPS) protocols for a PLP homopolymer incorporating SEQ ID: 2 as peptide sidechains. HEPES: 10 mM HEPES buffer (pH 7.4) with 0.1M EDTA and 2 mM DTT. Media: Dulbecco Modified Eagles Medium (high glucose) with 2 mM L-glutamine with 10% Fetal Bovine Serum, 1% Penicillin/Streptomycin. Scale: 50 μm.

FIG. 12: Demonstrates PEG-induced LLPS phase condensates of a PLP homopolymer incorporating SEQ ID: 4 as peptide sidechains. HEPES: 10 mM HEPES buffer (pH 7.4) with 0.1M EDTA and 2 mM DTT. Scale: 20 μm (left); 50 μm (middle and right).

FIG. 13: Cell permeability assay results for PLP homopolymers incorporating either SEQ ID: 4 (a-d) or SEQ ID: 2 (e-h) as peptide sidechains—scale: 20 μm. Cells: Striatal neurons grown in dish for 24 hours, treated with PLPs separately, then imaged 24 hours later—cells at a concentration of 1 μM with respect to dye (1:1 with PLPs). Bright spots in a and e show PLP; b and f show cell membrane; c and g show nucleus; and d and h provide an overlay.

FIG. 14: Results from liquid-liquid phase separation (LLPS)—with heparin—protocols for a PLP homopolymer incorporating SEQ ID: 4 as peptide sidechains. Test run with HEPES buffer: 10 mM HEPES buffer (pH 7.4) with 0.1M EDTA and 2 mM DTT. The dark background image (left) is a black and white representation of red channel output for rhodamine labelled polymers undergoing LLPS. The lighter background figure (middle) is from the brightfield images. The right-most figure depicts an overlay. Scale: 20 μm.

FIG. 15: Results from liquid-liquid phase separation (LLPS)—with heparin—protocols for a PLP homopolymer incorporating SEQ ID: 2 as peptide sidechains. Test run with HEPES buffer: 10 mM HEPES buffer (pH 7.4) with 0.1M EDTA and 2 mM DTT. The dark background image (left) is a black and white representation of red channel output for rhodamine labelled polymers undergoing LLPS. The lighter background figure (middle) is from the brightfield images. The right-most figure depicts an overlay. Scale: 20 μm.

FIG. 16: Results from Thioflavin T LLPS protocols for PLP homopolymers incorporating either SEQ ID: 4 (top four photos) or SEQ ID: 2 (bottom four photos) as peptide sidechains—scale: 50 μm. For each group of four, the image channels are: top left—red channel of rhodamine labeled PLP; top right—green channel of Thioflavin T; bottom left—brightfield; and bottom right—overlay of the 3 channels.

FIG. 17: SEM (top) and STEM (bottom) results for PLP homopolymer incorporating SEQ ID: 2 as peptide sidechains. Solution: water. Scale from left to right: 5 μm, 20 μm, 5 μm, 10 μm.

FIG. 18: Dry-state TEM results for PLP homopolymer incorporating SEQ ID: 2 as peptide sidechains after PLP was treated with heparin and heated in HEPES buffer. Scale: 1 μm (left and middle); 200 nm (right).

FIG. 19: Dry-state TEM results for K18 after K18 was treated with heparin and heated in PBS buffer. Scale: 1 μm.

FIG. 20: Circular dichroism (CD) results showing random coil formations before heparin was added (top) and beta sheet formation of polymers after addition of heparin (bottom) and binding to K18 Tau at 37° C. TP4: PLPs having SEQ ID: 4 as peptide sidechains; TP7: PLPs having SEQ ID: 2 as peptide side chains; TP12: PLPs having SEQ ID: 10 as peptide side chains.

FIG. 21: CD results showing beta sheet formation of peptides before and after addition of heparin at 37° C. P4 refers to a peptide characterized by SEQ ID: 4; P7 refers to a peptide characterized by SEQ ID: 2; P12 refers to a peptide characterized by SEQ ID: 10.

FIG. 22A-22B: Protein purification results of K18 Tau (FIG. 22A-22B, top) and BSA assay (FIG. 22B, bottom) to quantify protein.

FIG. 23: Two batches (split in two due to volume limits of column) of cleaved K18 over FPLC.

FIG. 24: Assessment of effect of concentration on K18 Tau kinetics. ThioT control: no Tau, no heparin, thioflavin T and PBS. Tau ThioT control: Tau, thioflavin T, no heparin, and PBS. ThioT heparin control: no Tau, thioflavin T, heparin, and PBS. Tau heparin control: Tau, no thioflavin T, heparin, and PBS. Tau: Tau, thioflavin T, heparin, and PBS. n=6. 1× Tau or Tau labels refer to 10 μM Tau final concentration in the well with 100 μl total volume.

FIG. 25: Concentration dependence of polymer activation of Thioflavin T. TP4: PLP homopolymer incorporating SEQ ID: 4 as peptide sidechains. TP7: PLP homopolymer incorporating SEQ ID: 2 as peptide sidechains. TP4 and TP7 controls: PLP homopolymer, thioflavin T, no heparin, and PBS. The remainder of controls were at baseline and were removed from this FIG. 25 to improve readability. ThioT control: no Tau, no heparin, thioflavin T and PBS. ThioT heparin control: no Tau, no PLPs, thioflavin T, heparin, and PBS. n=3.

FIG. 26: Addition of mimetic polymers to measure influence on aggregation of tau. TP4: PLP homopolymer incorporating SEQ ID: 4 as peptide sidechains. TP7: PLP homopolymer incorporating SEQ ID: 2 as peptide sidechains. The controls were at baseline and were removed from this FIG. 26 to improve readability. ThioT control: no Tau, no heparin, thioflavin T and PBS. Tau ThioT control: Tau, thioflavin T, no heparin, and PBS. ThioT heparin control: no Tau, thioflavin T, heparin, and PBS. Tau heparin control: Tau, no thioflavin T, heparin, and PBS. Tau: Tau, thioflavin T, heparin, and PBS. TP4 and TP7 controls: PLP homopolymer, thioflavin T, no heparin, and PBS. n=6. 1× Tau or Tau labels refer to 10 μM Tau final concentration in the well with 100 μl total volume.

FIG. 27: Addition of mimetic polymers to tau to evaluate fibrilization in the absence of heparin. TP4: PLP homopolymer incorporating SEQ ID: 4 as peptide sidechains. TP7: PLP homopolymer incorporating SEQ ID: 2 as peptide sidechains. The controls were at baseline and were removed from this FIG. 27 to improve readability. ThioT control: no Tau, no heparin, thioflavin T and PBS. Tau ThioT control: Tau, thioflavin T, no heparin, and PBS. ThioT heparin control: no Tau, thioflavin T, heparin, and PBS. Tau heparin control: Tau, no thioflavin T, heparin, and PBS. Tau: Tau, thioflavin T, heparin, and PBS. n=3. 1× Tau or tau labels refer to 10 μM tau final concentration in the well with 100 μl total volume.

FIG. 28: Influence of peptides on au kinetics. P4 refers to a peptide characterized by SEQ ID: 4. P7 refers to a peptide characterized by SEQ ID: 2. P12 refers to a peptide characterized by SEQ ID: 10. Tau: Tau, thioflavin T, heparin, and PBS. n=3.

FIG. 29: Addition of inhibitor polymer to tau to evaluate impact on au aggregation. TP12: PLP homopolymer incorporating SEQ ID: 10 as peptide sidechains. TP12 control: PLP homopolymer, no Tau, thioflavin T, heparin, and PBS. The remainder of controls were at baseline and were removed from this FIG. 29 to improve readability. ThioT control: no Tau, no heparin, thioflavin T and PBS. Tau ThioT control: Tau, thioflavin T, no heparin, and PBS. ThioT heparin control: no Tau, thioflavin T, heparin, and PBS. Tau heparin control: Tau, no thioflavin T, heparin, and PBS. Tau: Tau, thioflavin T, heparin, and PBS. n=3.

FIG. 30: Impact of inhibitor polymer at various concentrations on tau kinetics. TP12: PLP homopolymer incorporating SEQ ID: 10 as peptide sidechains. TP12 control: PLP homopolymer, no Tau, thioflavin T, heparin, and PBS. The remainder of controls were at baseline and were removed from this FIG. 30 to improve readability. ThioT control: no Tau, no heparin, thioflavin T and PBS. Tau ThioT control: Tau, thioflavin T, no heparin, and PBS. ThioT heparin control: no Tau, thioflavin T, heparin, and PBS. Tau heparin control: Tau, no thioflavin T, heparin, and PBS. Tau: Tau, thioflavin T, heparin, and PBS. n=3.

FIG. 31: Repeat of impact of inhibitor polymer at various concentrations on tau kinetics. TP12: PLP homopolymer incorporating SEQ ID: 10 as peptide sidechains. TP12 control: PLP homopolymer, no Tau, thioflavin T, heparin, and PBS. The remainder of controls were at baseline and were removed from this FIG. 31 to improve readability. ThioT control: no Tau, no heparin, thioflavin T and PBS. Tau ThioT control: Tau, thioflavin T, no heparin, and PBS. ThioT heparin control: no Tau, thioflavin T, heparin, and PBS. Tau heparin control: Tau, no thioflavin T, heparin, and PBS. Tau: Tau, thioflavin T, heparin, and PBS. n=3.

FIG. 32: Polymerization kinetics for a PLP homopolymer incorporating SEQ ID: 10 (“TP12”) as peptide side chains.

FIG. 33: SEC-MALS results depicting polymer characteristics of a PLP homopolymer incorporating SEQ ID: 10 (“TP12”) as peptide side chains. Mn: 25.160 kDa (±0.671%); Mw: 26.050 kDa (±0.671%); PDI: 1.035 (±0.949%).

FIG. 34: SDS-PAGE results for rhodamine labelled PLPs. TP4 refers to a PLP homopolymer incorporating SEQ ID: 4. TP7 refers to a PLP homopolymer incorporating SEQ ID: 2.

FIG. 35A-35B: A schematic demonstrating the characteristics of the PLP platform. Protein-like polymers (PLPs) are peptide-brush polymers that fold into globular proteomimetics that: (a) are flexible and adaptable (b) are proteolytically resistant, (c) are cell-penetrating for cytosolic distribution, and (d) exhibit multivalent target engagement (FIG. 35A). They can be readily tuned for desired physical or biological behavior. (e) Synthesis of PLPs using graft-through ring-opening metathesis polymerization (ROMP) and (f) reversible addition-fragmentation chain transfer polymerization (RAFT) (FIG. 35B).

### STATEMENTS REGARDING CHEMICAL COMPOUNDS AND NOMENCLATURE

The following abbreviations are used herein: RP-HPLC refers to reverse-phase high performance liquid chromatography; ESI-MS refers to electrospray ionization mass spectrometry; SEC-MALS refers to size-exclusion chromatography coupled with multiangle light scattering; PLP refers to protein-like polymer; SPPS refers to solid-phase peptide synthesis; TEM refers to transmission electron microscopy; STEM refers to scanning TEM; SE or SEM refers to scanning electron microscopy; CD refers to circular dichroism; FPLC refers to fast protein liquid chromatography; and DP refers to degree of polymerization.

In an embodiment, a peptide, a polymer, or a composition (e.g., formulation) of the invention is isolated or purified. In an embodiment, an isolated or purified peptide, polymer, or composition (e.g., formulation) is at least partially isolated or purified as would be understood in the art. In an embodiment, the peptide, polymer, or composition (e.g., formulation) of the invention has a chemical purity of at least 95%, optionally for some applications at least 99%, optionally for some applications at least 99.9%, optionally for some applications at least 99.99%, and optionally for some applications at least 99.999% pure. The invention includes isolated and purified compositions of any of the brush block polymers described herein including the peptide brush and block copolymers and brush and brush block copolymers having one or more side chains comprising the peptide analogues, derivative, variants or fragments.

As used herein, the term “polymer” refers to a molecule composed of repeating structural units connected by covalent chemical bonds often characterized by a substantial number of repeating units (e.g., equal to or greater than 3 repeating units, optionally, in some embodiments equal to or greater than 5 repeating units, in some embodiments greater or equal to 10 repeating units) and a high molecular weight (e.g., greater than or equal to 1 kDa, in some embodiments greater than or equal to 5 kDa or greater than or equal to 50 kDa). Polymers are commonly the polymerization product of one or more monomer precursors. The term polymer includes homopolymers, or polymers consisting essentially of a single repeating monomer subunit. The term polymer also includes copolymers which are formed when two or more different types of monomers are linked in the same polymer. Copolymers may comprise two or more monomer subunits (e.g., 3 or more monomer subunits, 4 or more monomer subunits, 5 or more monomer subunits, or 6 or more monomer subunits), and include random, block, brush, brush block, alternating, segmented, grafted, tapered and other architectures. In some embodiments, copolymers of the invention comprise from 2 to 10 different monomer subunits. Useful polymers include organic polymers that may be in amorphous, semi-amorphous, crystalline or semi-crystalline states. Cross linked polymers having linked monomer chains are useful for some applications, for example linked by one or more disulfide linkages. The invention provides polymers comprising therapeutic agents, such as brush polymers having at least a portion of the repeating units comprising polymer side chains such as peptide side chains.

An “oligomer” refers to a molecule composed of repeating structural units connected by covalent chemical bonds often characterized by a number of repeating units less than that of a polymer (e.g., equal to or less than 3 repeating units) and a lower molecular weights (e.g., less than or equal to 1,000 Da) than polymers. Oligomers may be the polymerization product of one or more monomer precursors.

A “peptide” or “oligopeptide” herein are used interchangeably and refer to a polymer of repeating structural units connected by a peptide bond. Typically, the repeating structural units of the peptide are amino acids including naturally occurring amino acids, non-naturally occurring amino acids, analogues of amino acids or any combination of these. The number of repeating structural units of a peptide, as understood in the art, are typically less than a “protein”, and thus the peptide often has a lower molecular weight than a protein. In some embodiments, a peptide has a chain length of 3 to 150 amino acids, optionally 3 to 100 amino acids, optionally 5 to 50 amino acids, and optionally 5 to 30 amino acids.

“Block copolymers” are a type of copolymer comprising blocks or spatially segregated domains, wherein different domains comprise different polymerized monomers, for example, including at least two chemically distinguishable blocks. Block copolymers may further comprise one or more other structural domains, such as hydrophobic groups, hydrophilic groups, etc. In a block copolymer, adjacent blocks are constitutionally different, i.e., adjacent blocks comprise constitutional units derived from different species of monomer or from the same species of monomer but with a different composition or sequence distribution of constitutional units. Different blocks (or domains) of a block copolymer may reside on different ends or the interior of a polymer (e.g., [A][B]), or may be provided in a selected sequence ([A][B][A][B]). “Diblock copolymer” refers to block copolymer having two different polymer blocks. “Triblock copolymer” refers to a block copolymer having three different polymer blocks, including compositions in which two non-adjacent blocks are the same or similar. “Pentablock” copolymer refers to a copolymer having five different polymer including compositions in which two or more non-adjacent blocks are the same or similar.

“Statistical copolymers,” also generally known in the art as “random copolymers,” are copolymers in which the ordering of backbone groups is dictated by reaction kinetics. Statistical copolymers generally are antithetical to block copolymers.

“Polymer backbone group” or “polymer backbone subunit” refers to groups that are covalently linked to make up a backbone of a polymer, such as a block copolymer. Polymer backbone groups may be linked to side chain groups, such as polymer side chain groups. Some polymer backbone groups useful in the present compositions are derived from polymerization of a monomer selected from the group consisting of a substituted or unsubstituted norbornene, olefin, cyclic olefin, norbornene anhydride, cyclooctene, cyclopentadiene, styrene, acrylamide, and acrylate. Some polymer backbone groups useful in the present compositions are obtained from a ring opening metathesis polymerization (ROMP) reaction. Polymer backbones may terminate in a range of backbone terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³²—COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane, acrylamide, acrylate, or catechol; wherein each of R³⁰—R⁴² is independently hydrogen, C₁-C₁₀ alkyl or C₅-C₁₀ aryl.

“Polymer side chain group” (also sometimes referred to herein as “substituent,” e.g., with respect to R¹) refers to a group covalently linked (directly or indirectly) to a polymer backbone group that comprises a polymer side chain, optionally imparting steric properties to the polymer. In an embodiment, for example, a polymer side chain group is characterized by a plurality of repeating units having the same, or similar, chemical composition. A polymer side chain group may be directly or indirectly linked to the polymer back bone groups. In some embodiments, polymer side chain groups provide steric bulk and/or interactions that result in an extended polymer backbone and/or a rigid polymer backbone. Some polymer side chain groups useful in the present compositions include unsubstituted or substituted peptide groups. Some polymer side chain groups useful in the present compositions comprise repeating units obtained via anionic polymerization, cationic polymerization, free radical polymerization, group transfer polymerization, or ring-opening polymerization. A polymer side chain may terminate in a wide range of polymer side chain terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³1R³²—COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane, acrylamide, acrylate, or catechol; wherein each of R³⁰—R⁴² is independently hydrogen or C₁-C₅ alkyl.

As used herein, the term “polymer segment” (e.g., first polymer segment, second polymer segment, etc.) refers to a section (e.g., portion) of the polymer comprising a particular monomer or arrangement of monomers. A polymer segment can be a homopolymer or a copolymer. In embodiments where a polymer segment is a copolymer, the copolymer can exist in any suitable arrangement of monomers (e.g., random, block, brush, brush block, alternating, segmented, grafted, tapered, statistical and other architectures). In some embodiments, the polymer segments are homopolymers, random copolymers, statistical copolymers, or block copolymers. Any polymer (e.g., brush polymer) described herein can have a single polymer segment or multiple polymer segments. In embodiments where the polymer has multiple polymer segments, the polymer segments can exist in any suitable arrangement (random, block, brush, brush block, alternating, segmented, grafted, tapered, statistical, and other architectures).

As used herein, the term “degree of polymerization” refers to the average number of monomer units per polymer chain. For example, for certain polymers described herein, comprising B¹, B², and/or B³ backbone units, the degree of polymerization would be represented by the sum total of B¹, B², and B³ backbone units. Since the degree of polymerization can vary from polymer to polymer, the degree of polymerization is generally represented by an average.

As used herein, the term “brush polymer” refers to a polymer comprising repeating units each independently comprising a polymer backbone group covalently linked to at least one polymer side chain group. A brush polymer may be characterized by brush density which refers to the percentage of the repeating units comprising polymer side chain groups. Brush polymers of certain aspects are characterized by a brush density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects are characterized by a brush density selected from the range 50% to 100%, optionally some embodiments a density selected from the range of 75% to 100%, or optionally for some embodiments a density selected from the range of 90% to 100%. Brush polymers, such as the polymers disclosed herein (e.g., a polymer of formula (1)), can be prepared by any suitable methods including, “grafting from” methods, “grafting onto” methods, “grafting through” methods, or any combination thereof. Such suitable methods can include, for example, ring opening metathesis polymerization (ROMP) synthetic pathways and/or non-ROMP synthetic pathways, such as, by way of example, reversible addition fragmentation chain transfer (RAFT) polymerization, stable free radical mediated polymerization and atom transfer radical polymerization (ATRP).

As used herein, the term “peptide density” refers to the percentage of monomer units in the polymer chain which have a peptide covalently linked thereto, and such “peptide density” can be calculated generally for all peptides or for a specific peptide. The percentage is based on the overall sum of monomer units in the polymer chain. For example, for certain polymers described herein, the density of peptide P¹ (or percentage of monomer units comprising peptide P¹) in a polymer having m repeat units of peptide P¹, n repeat units of B²—R¹, and o repeat units of peptide P², is represented by the formula:

$$\frac{m}{m + n + o} \times 100 ,$$

where each variable refers to the number of monomer units of that type in the polymer chain. Polymers of certain aspects are characterized by a peptide density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Polymers of certain aspects are characterized by a peptide density selected from the range 50% to 100%, optionally some embodiments a density selected from the range of 75% to 100%, or optionally for some embodiments a density selected from the range of 90% to 100%. In some embodiments, the brush density is equal to the peptide density.

In an aspect, the polymer side chain groups (e.g., also termed substituents herein) can have any suitable spacing on the polymer backbone. Typically, the space between adjacent polymer side chain groups is from 3 angstroms to 30 angstroms, and optionally 5 to 20 angstroms and optionally 5 to 10 angstroms. By way of illustration, in certain embodiments having a brush density of 100%, the polymer side chain groups typically are spaced 6±5 angstroms apart on the polymer backbone. In some embodiments the brush polymer has a high a brush density (e.g., greater than 70%), wherein the polymer side chain groups are spaced 5 to 20 angstroms apart on the polymer backbone.

As used herein, the term “sequence homology” or “sequence identity” means the proportion of amino acid matches between two amino acid sequences of interest in two different peptides considering the ordering of the amino acids. Matches occur when amino acids are in the same order in one peptide compared to the other peptide. When sequence homology is expressed as a percentage, e.g., 50%, the percentage denotes the fraction of matches over the length of sequence that is compared to some other sequence, considering the amino acid order. Gaps (in either of the two sequences) are permitted to maximize matching; for example, wherein gap lengths of 5 amino acids or less, optionally 3 amino acids or less, are usually used. In other words, a sequence having 75% or greater sequence identity to an amino acid sequence with 9 amino acids can indicate that the 9 amino acid sequence can have one or two point mutations (i.e., amino acid change), one or two amino acid deletions, one or two amino acid additions, one point mutation and one amino acid deletion, or one point mutation and one amino acid addition. Even with two such amino acids being different, 7 out of 9 amino acids still match in the correct order, such that there is greater than 75% sequence identity. For clarity, the analysis of whether there sequence homology between two amino acid sequences of interest is conducted with respect to a particular portion of one peptide or protein (i.e., a first amino acid sequence of interest) relative to a particular portion of another peptide or protein (i.e., a second amino acid sequence of interest), and is not conducted relative to all amino acids present in a peptide or protein (i.e., the analysis does not include amino acids outside of the particular amino acid sequence of interest).

As used herein, the term “amino acid composition similarity” or “amino acid similarity” means the proportion of amino acid matches between two amino acid sequences of interest in two different peptides regardless of the ordering of the amino acids. Matches occur when amino acids are present in both amino acid sequences regardless of order. When amino acid composition similarity is expressed as a percentage, e.g., 50%, the percentage denotes the fraction of matches over the length of sequence that is compared to some other sequence, regardless of amino acid order. Gaps (in either of the two sequences) are permitted to maximize matching; for example, wherein gap lengths of 5 amino acids or less, optionally 3 amino acids or less, are usually used. By way of example, if two amino acid sequences each containing ten amino acids have three amino acids in common, in any order, then there is 30% amino acid composition similarity between the sequences. For clarity, the analysis of whether there is amino acid composition similarity between two amino acid sequences of interest is conducted with respect to a particular portion of one peptide or protein (i.e., a first amino acid sequence of interest) relative to a particular portion of another peptide or protein (i.e., a second amino acid sequence of interest), and is not conducted relative to all amino acids present in a peptide or protein (i.e., the analysis does not include amino acids outside of the particular amino acid sequence of interest).

The term “fragment” refers to a portion, but not all of, a composition or material, such as a peptide composition or material. In an embodiment, a fragment of a peptide refers to 50% or more of the sequence of amino acids, optionally 70% or more of the sequence of amino acids and optionally 90% or more of the sequence of amino acids.

“Polymer blend” refers to a mixture comprising at least one polymer, such as a brush polymer, e.g., brush block copolymer, and at least one additional component, and optionally more than one additional component. In some embodiments, for example, a polymer blend of the invention comprises a first brush copolymer and one or more addition brush polymers having a composition different than the first brush copolymer. In some embodiments, for example, a polymer blend of the invention further comprises one or more additional brush block copolymers, homopolymers, copolymers, block copolymers, brush block copolymers, oligomers, solvent, small molecules (e.g., molecular weight less than 500 Da, optionally less than 100 Da), or any combination of these. Polymer blends useful for some applications comprise a first brush polymer, and one or more additional components comprising polymers, block copolymers, brush polymers, linear block copolymers, random copolymers, homopolymers, or any combinations of these. Polymer blends of the invention include mixture of two, three, four, five and more polymer components.

As used herein, the term “compound” can be used to refer to any of the peptides or polymers described herein. Alternatively, or additionally, the term compound can refer to any of the synthetic precursors, reagents, additives, excipients, etc. used in preparation of or formulation with the peptides or polymers described herein.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

As used herein, the term “substituted” generally refers to a compound wherein a hydrogen is replaced by another functional group, unless otherwise contradicted by context.

Unless otherwise specified, the term “average molecular weight” or “molecular weight” refers to number average molecular weight. Number average molecular weight is the defined as the total weight of a sample volume divided by the number of molecules within the sample. As is customary and well known in the art, peak average molecular weight and weight average molecular weight may also be used to characterize the molecular weight of the distribution of polymers within a sample.

As used herein, the term “K18” refers to a recombinant Tau protein fragment having the four domains responsible for microtubule binding and amyloid fibril formation. The 129-residue-long chain is considered the core peptide of Tau, or the active Tau monomer.

As used herein, “mimic,” “mimicking,” “mimetic,” and grammatically equivalent variations in reference to a compound, oligomer, and/or polymer mimicking a given species (e.g., “proteomimetic”), such as one or more oligo- or poly-peptides (e.g., proteins), means that the compound, oligomer, and/or polymer has a portion with a similar and/or corresponding amino acid sequence to a portion of the given species. In some aspects, the similar and/or corresponding portion typically relates to there being a certain level of sequence homology and/or amino acid composition similarity between the given species and the one or more oligo- or poly-peptides (e.g., proteins). In aspects, a mimetic refers to a material capable of imitating key structures and/or functions of a peptide or protein. Mimetics may be synthetically produced and modified to comprise specific properties depending on desired outcome, including variable size, greater stability, greater affinity, protease-resistance and improved solubility. In aspects, mimetic refers to a protein-like polymer (PLP) designed to engage proteins and the quality control machinery within cells. Callmann C E et al., Poly(peptide): Synthesis, Structure, and Function of Peptide-Polymer Amphiphiles and Protein-like Polymers. Acc Chem Res 2020; 53:400-13; Gianneschi N C et al., Biomolecular Densely Grafted Brush Polymers: Oligonucleotides, Oligosaccharides and Oligopeptides. Angew Chemie Int Ed 2020; Blum A P, Kammeyer J K, Gianneschi N C, each of which is incorporated by reference herein in its entirety, and more specifically to facilitate the understanding of PLPs, to the extent not inconsistent with the description herein.

In aspects of the invention, a mimetic may be modified to comprise a residue-specific modification, a peptide backbone modification, an N-terminal modification, a C-terminal modification, or any combination thereof. In examples, the modification may improve peptide stability, alter peptide structure, incorporate imaging and/or detection agents, improve solubility, enhance non-specific enzyme resistance, reduce steric hindrance, increase cellular penetration, improve binding affinities to targets, enhance safety, or any combination thereof. For example, a modification may include one or more of: biotin labeling, contrast agent labeling such as Gd-DOTA labeling, fluorescent dye labeling such as cyanine labeling, fluorescein and 7-methoxycoumarin acetic acid labeling, dansyl and/or 2,4-dinitrophenyl labeling, EDANS labeling, coumarin labeling, and/or rhodamine labeling, one or more point mutations, introduction of one or more spacers, isotopic labeling, introduction of one or more chelating agents, acetylation, amidation, methylation, palmitylation, hydroxylation, glycosylation, sulfation and sulfonation, esterification, phosphorylation, peptide stapling, lipidation, cyclization, or any combination thereof.

As used herein, the phrase “charge modulating domain” refers to one or more amino acids added to the peptide sequences described herein to modulate the charge of the peptide. For example, the charge modulating domain can be a TAT sequence, a glycine-serine domain, a cationic residue domain, or a combination thereof, or optionally a glycine-serine domain, a cationic residue domain, or a combination thereof. In certain embodiments, the charge modulating domain has from 2 to 7 amino acid residues. The 2 to 7 amino acids can be added in a single block containing from 2 to 7 amino acid residues or more than one block containing from 1 to 6 amino acid residues. In some embodiments, the charge modulating domain is a cationic residue domain having from 2 to 7 amino acid residues selected from lysine, arginine, histidine, or a combination thereof. In some embodiments, the charge modulating domain comprises an aspartic acid residue. Generally, the charge modulating domain modulates the charge of the peptide to have a net positive charge. Without wishing to be bound by any particular theory, it is believed that the net positive charge increases the cellular uptake of the peptide or polymer comprising the peptide. The overall charge of the peptide or copolymer comprising the peptide can be determined by any suitable means. For example, the overall charge can be determined by (i) structural analysis of the functional residues on the peptide sequence and their respective pKa, (ii) physical characterization by measuring the zeta potential, and/or (iii) by virtue of the material moving towards a negative pole in an electrophoresis polymer gel. In certain embodiments, the overall charge of the peptide or copolymer comprising the peptide is determined by measuring the zeta potential.

As used herein, a “degrader agent” or “degron” refers to a class of agents capable of directly or indirectly facilitating the regulation of protein degradation. For example, the regulation may comprise promoting degradation, inhibiting degradation, increasing the rate of degradation, and/or decreasing the rate of degradation of a protein. In embodiments, such as wherein the degrader agent is incorporated in a PLP, the degrader agent facilitates specific degradation of a targeted protein. In some aspects of the invention, the degrader agent facilitates ubiquitin recruitment. Without subscribing to a particular theory, it is believed that in aspects wherein the degrader agent facilitates ubiquitin recruitment, the degrader agent participates in the polyubiquitination process to target proteins, or fragments thereof, for degradation by a proteasome. In these aspects, the degrader agent may be referred to herein as a “proteasome recruiter.” In embodiments, the degrader agent comprises a proteasome-targeting chimera (“PROTAC”). In aspects, the degrader agent comprises any suitable number of amino acid units so long as the peptide comprises a sequence having 75% or greater (e.g., 75% or greater, 80% or greater, 85% or greater, 90% or greater, 95% or greater, or 100%) sequence identity of SEQ ID 11: (ALAPYIP) or SEQ ID: 12 (ALAPYIPR).

In some embodiments, the degrader agent is a degrader peptide or component or fragment thereof. In embodiments, the degrader agent may be a degrader peptide having a chain length of 3 to 150 amino acids, optionally of 3 to 100 amino acids, optionally 5 to 50 amino acids, optionally 5 to 20 amino acids, and optionally 4 to 10 amino acids. In some embodiments, the degrader agent may comprise a small molecule degrader. In examples, the small molecule degrader comprises a low molecular weight organic compound having a molecular weight of less than or equal to 2 kDa, optionally less than or equal to 1.5 kDa, or optionally less than or equal to 1 kDa. In some embodiments, the degrader agent is characterized by molecular weight between 100 Da and 2000 Da. In some embodiments, the degrader agent is characterized by molecular weight between 250 Da and 1500 Da.

As used herein, “HYDRAC” refers to Heterofunctional polYmeric DegRading Chimeras (HYDRACs). In aspects, HYDRACS are a subclass of PLPs which contain heterologous side chains with distinct functionalities, wherein one domain binds to a protein of interest and a second targets it for degradation.

As used herein, the terms “alkylene” and “alkylene group” are used synonymously and refer to a divalent group derived from an alkyl group as defined herein. The invention includes compounds having one or more alkylene groups. Alkylene groups in some compounds function as linking and/or spacer groups. Compounds of the invention may have substituted and/or unsubstituted C₁-C₂₀ alkylene, C₁-C₁₀ alkylene and C₁-C₅ alkylene groups, for example, as one or more linking groups (e.g., L¹, L²).

As used herein, the terms “cycloalkylene” and “cycloalkylene group” are used synonymously and refer to a divalent group derived from a cycloalkyl group as defined herein. The invention includes compounds having one or more cycloalkylene groups. Cycloalkyl groups in some compounds function as linking and/or spacer groups. Compounds of the invention may have substituted and/or unsubstituted C₃-C₂₀ cycloalkylene, C₃-C₁₀ cycloalkylene and C₃-C₅ cycloalkylene groups, for example, as one or more linking groups (e.g., L¹, L²).

As used herein, the terms “arylene” and “arylene group” are used synonymously and refer to a divalent group derived from an aryl group as defined herein. The invention includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as linking and/or spacer groups. Arylene groups in some compounds function as chromophore, fluorophore, aromatic antenna, dye and/or imaging groups. Compounds of the invention include substituted and/or unsubstituted C₃-C₃₀ arylene, C₃-C₂₀ arylene, C₃-C₁₀ arylene and C₁-C₅ arylene groups, for example, as one or more linking groups (e.g., L¹, L²).

As used herein, the terms “heteroarylene” and “heteroarylene group” are used synonymously and refer to a divalent group derived from a heteroaryl group as defined herein. The invention includes compounds having one or more heteroarylene groups. In some embodiments, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as linking and/or spacer groups. Heteroarylene groups in some compounds function as chromophore, aromatic antenna, fluorophore, dye and/or imaging groups. Compounds of the invention include substituted and/or unsubstituted C₃-C₃₀ heteroarylene, C₃-C₂₀ heteroarylene, C₁-C₁₀ heteroarylene and C₃-C₅ heteroarylene groups, for example, as one or more linking groups (e.g., L¹, L²).

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as linking and/or spacer groups. Compounds of the invention include substituted and/or unsubstituted C₂-C₂₀ alkenylene, C₂-C₁₀ alkenylene and C₂-C₅ alkenylene groups, for example, as one or more linking groups (e.g., L¹, L²).

As used herein, the terms “cycloalkenylene” and “cycloalkenylene group” are used synonymously and refer to a divalent group derived from a cycloalkenyl group as defined herein. The invention includes compounds having one or more cycloalkenylene groups. Cycloalkenylene groups in some compounds function as linking and/or spacer groups. Compounds of the invention include substituted and/or unsubstituted C₃-C₂₀ cycloalkenylene, C₃-C₁₀ cycloalkenylene and C₃-C₅ cycloalkenylene groups, for example, as one or more linking groups (e.g., L¹, L²).

As used herein, the terms “alkynylene” and “alkynylene group” are used synonymously and refer to a divalent group derived from an alkynyl group as defined herein. The invention includes compounds having one or more alkynylene groups. Alkynylene groups in some compounds function as linking and/or spacer groups. Compounds of the invention include substituted and/or unsubstituted C₂-C₂₀ alkynylene, C₂-C₁₀ alkynylene and C₂-C₅ alkynylene groups, for example, as one or more linking groups (e.g., L¹, L²).

As used herein, the term “halo” refers to a halogen group such as a fluoro (—F), chloro (—Cl), bromo (—Br), iodo (—I) or astato (—At).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

As used herein, the term “polyalkoxyalkyl” refers to a substituent of the formula alkyl-(alkoxy)ₙ-alkoxy wherein n is an integer from 1 to 10, preferably 1 to 4, and more preferably for some embodiments 1 to 3.

Amino acids include glycine, alanine, valine, leucine, isoleucine, methionine, proline, phenylalanine, tryptophan, asparagine, glutamine, glycine, serine, threonine, serine, threonine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids. Peptides are comprised of two or more amino acids connected via peptide bonds.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, or 7-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO—refers to CH₃O—. Compositions of some embodiments of the invention comprise alkyl groups as terminating groups, such as polymer backbone terminating groups and/or polymer side chain terminating groups.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenyl groups can also carry alkyl groups. Cycloalkenyl groups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms. Compositions of some embodiments of the invention comprise alkenyl groups as terminating groups, such as polymer backbone terminating groups and/or polymer side chain terminating groups.

Aryl groups include groups having one or more 5-, 6- or 7-member aromatic rings, including heterocyclic aromatic rings. The term heteroaryl specifically refers to aryl groups having at least one 5-, 6- or 7-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those which are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-membered ring and one or more additional five- or six-membered aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents. Compositions of some embodiments of the invention comprise aryl groups as terminating groups, such as polymer backbone terminating groups and/or polymer side chain terminating groups.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms. Compositions of some embodiments of the invention comprise arylalkyl groups as terminating groups, such as polymer backbone terminating groups and/or polymer side chain terminating groups.

As to any of the groups described herein which contain one or more substituents, it is understood that such groups do not contain any substitution or substitution patterns which are sterically impractical and/or synthetically non-feasible. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others: halogen, including fluorine, chlorine, bromine or iodine; pseudohalides, including —CN;

—COOR where R is a hydrogen or an alkyl group or an aryl group and more specifically where R is a methyl, ethyl, propyl, butyl, or phenyl group all of which groups are optionally substituted;

—COR where R is a hydrogen or an alkyl group or an aryl group and more specifically where R is a methyl, ethyl, propyl, butyl, or phenyl group all of which groups are optionally substituted;

—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;

—SR, where R is hydrogen or an alkyl group or an aryl group and more specifically where R is hydrogen, methyl, ethyl, propyl, butyl, or a phenyl group, which are optionally substituted;

—SO₂R, or —SOR where R is an alkyl group or an aryl group and more specifically where R is a methyl, ethyl, propyl, butyl, or phenyl group, all of which are optionally substituted;

—OCOOR where R is an alkyl group or an aryl group;

—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms;

—OR where R is H, an alkyl group, an aryl group, or an acyl group all of which are optionally substituted. In a particular example R can be an acyl yielding —OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

As to any of the above groups which contain one or more substituents, it is understood that such groups do not contain any substitution or substitution patterns which are sterically impractical and/or synthetically non-feasible.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, which is combined with buffer prior to use.

Thus, the compounds, oligomers, or polymers disclosed herein may exist as salts, such as with pharmaceutically acceptable acids. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds, oligomers, or polymers disclosed herein can exist in unsolvated forms as well as solvated forms, including hydrated forms. In general, the solvated forms are equivalent to unsolvated forms and are encompassed within the scope of the present invention. Certain compounds disclosed herein may exist in multiple crystalline or amorphous forms. In general, all physical forms are equivalent for the uses contemplated herein and are intended to be within the scope of the disclosed compounds, oligomers, or polymers.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or D- or L-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms. Isomers include structural isomers and stereoisomers such as enantiomers.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

As is customary and well known in the art, hydrogen atoms in formulas (FX1) and (S1a)-(S2c) are not always explicitly shown, for example, hydrogen atoms bonded to the carbon atoms of aromatic, heteroaromatic, and alicyclic rings are not always explicitly shown in formulas (FX1) and (S1a)-(S2c). The structures provided herein, for example in the context of the description of formulas (FX1) and (S1a)-(S2c) and schematics and structures in the drawings, are intended to convey to one of reasonable skill in the art the chemical composition of compounds of the methods and compositions of the invention, and as will be understood by one of skill in the art, the structures provided do not indicate the specific positions and/or orientations of atoms and the corresponding bond angles between atoms of these compounds.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to a subject, such as a patient in need of treatment; slowing in the rate of degeneration or decline; making the final point of degeneration less debilitating; improving a subject's physical or mental well-being. The treatment or amelioration of symptoms can be based on objective or subjective parameters; including the results of a physical examination, neuropsychiatric exams, and/or a psychiatric evaluation.

An “effective amount” is an amount sufficient to accomplish a stated purpose (e.g., achieve the effect for which it is administered, treat a disease, reduce enzyme activity, increase enzyme activity, reduce transcriptional activity, increase transcriptional activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist (inhibitor) required to decrease the activity of an enzyme or protein (e.g., transcription factor) relative to the absence of the antagonist. An “activity increasing amount,” as used herein, refers to an amount of agonist (activator) required to increase the activity of an enzyme or protein (e.g., transcription factor) relative to the absence of the agonist. A “function disrupting amount,” as used herein, refers to the amount of antagonist (inhibitor) required to disrupt the function of an enzyme or protein (e.g., transcription factor) relative to the absence of the antagonist. A “function increasing amount,” as used herein, refers to the amount of agonist (activator) required to increase the function of an enzyme or protein (e.g., transcription factor) relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “inhibition,” “inhibit,” “inhibiting,” and grammatically equivalent variations in reference to a compound, oligomer, and/or polymer inhibiting aggregation means disrupting, preventing, or otherwise negatively affecting (e.g., decreasing) the ability of a given species, such as one or more oligo- or poly-peptides (e.g., proteins), to bind together (e.g., noncovalently) or otherwise associate relative to the level of association of such species in the absence of the compound, oligomer, and/or polymer. As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g., agonist) interaction means positively affecting (e.g., increasing) the activity or function of the protein.

The term “modulator” refers to a composition that increases or decreases the level of a target molecule or the function of a target molecule.

“Patient”, “subject”, or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a mammal. In some embodiments, a patient is a mouse. In some embodiments, a patient is an experimental animal. In some embodiments, a patient is a rat. In some embodiments, a patient is a test animal.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). In embodiments, administration includes direct administration to a tumor. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent or chemotherapeutic). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212, 162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der waal's bond(s)/interactions, hydrogen bond(s), polar bond(s), or combinations or mixtures thereof).

As used herein, the term “about” means a range of values including the specified value, which a person of ordinary skill in the art would consider reasonably similar to the specified value. In some aspects, about means within a standard deviation using measurements generally acceptable in the art. In some aspects, about means a range extending to +/−10% of the specified value. In embodiments, about means the specified value.

Various polymers disclosed herein are characterized, in part, by the relative amounts of distinct functional side chains present in the polymer. In aspects, the relative amounts of distinct functional side chains is represented as an average ratio defined herein as “peptide ratio.” Since the degree of polymerization can vary from polymer to polymer, the composition of monomers (and functional side chains of said monomers) can also vary. Therefore, the peptide ratio should be understood as an average. It will be appreciated by one having skill in the art that polymerization methods and subsequent analysis methods are subject to random, experimental error, and the peptide ratios should therefore be read to encompass reasonable variations from the stated value. Specifically, in some aspects, peptide ratios associated with functional side chains of polymers include variations of 20% of the stated ratio. In keeping with this aspect, a P¹:P² ratio of 2:1 includes variations of P¹:P² ratios of 1.6:1 to 2.4:1 (e.g., 1.6:1, 1.8:1, 2:1, 2.2:1, 2.4:1) and 2:0.8 to 2:1.2 (e.g., 2:0.8, 2:0.9, 2:1, 2:1.1, 2:1.2). In some aspects, peptide ratios associated with functional side chains of polymers include variations of 10% of the stated ratio. In some aspects, peptide ratios associated with functional side chains of polymers include variations of ±5% of the stated ratio. In some aspects, peptide ratios associated with functional side chains of polymers include variations of ±1% of the stated ratio.

As used herein, the term “acceleration,” “accelerate,” “accelerating,” and grammatically equivalent variations in reference to a compound, oligomer, and/or polymer accelerating aggregation means promoting, facilitating, or otherwise positively affecting (e.g., increasing or speeding up) the ability of a given species, such as one or more oligo- or poly-peptides (e.g., proteins), to bind together (e.g., noncovalently) or otherwise associate relative to the level of association of such species in the absence of the compound, oligomer, and/or polymer. Acceleration of aggregation is relevant, for example, in the context of speeding up aggregation past a toxic species, such as nanofibirils, towards higher order aggregates that are less toxic, so as to reduce overall toxicity of the system.

As used herein, the term “bind,” “binding,” and grammatically equivalent variations in reference to a compound, oligomer, and/or polymer binding to a given species, such as one or more oligo- or poly-peptides (e.g., proteins), means that the compound, oligomer, and/or polymer makes multiple noncovalent bonds to the given species. In some aspects, the multiple noncovalent bonds are possible because the compound, oligomer, and/or polymer has a portion with a similar and/or corresponding amino acid sequence to a portion of the given species.

As used herein, the phrase “at least a portion of each instance of P¹ independently comprises at least” a specified percentage of amino acid composition similarity and/or sequence homology, and similar phrasing, refers to a discrete segment of P¹ having the indicated amino acid composition similarity and/or sequence homology. For clarity, the specified percentage is not determined by references to single amino acids taken from unrelated segments of P¹.

As used herein, the phrase “at least a portion of each instance of P¹ independently is or comprises at least one of” specified amino acid sequences, and similar phrasing, means that each P¹ can be any of the specified amino acid sequences or any combination of the specified amino acid sequences.

As used herein, “triggering an organism's ubiquitination cellular machinery,” or similar phrasing, means activating an organism's molecular components, such as enzymes, to cause ubiquitination of an oligo- or poly-peptide (e.g., protein) of interest, such as tau and/or microtubulin.

As used herein, “liquid-liquid phase separation” (LLPS) is where an initially homogeneous solution condenses into droplets upon exceeding a critical saturation concentration, forming a dense (protein-rich) phase of droplets within a dilute (protein-poor) phase.

As used herein, an “aggregative region” of an oligo- or poly-peptide (e.g., protein) is the portion thereof that is prone to aggregate with the same or similar oligo- or poly-peptides to form, for example, aggregated species up to and including amyloids (e.g., of tau and/or microtubulin proteins).

As used herein, “metaphilic” means a compound, oligomer, or polymer that is transiently amphiphilic, such as by way of a hydrophobic backbone with hydrophilic side chains. In some aspect, the metaphilicity leads to globular but fluxional structures, which may be useful for penetrating cell walls.

### DETAILED DESCRIPTION

In the following description, numerous specific details of the devices, device components and methods of the present invention are set forth in order to provide a thorough explanation of the precise nature of the invention. It will be apparent, however, to those of skill in the art that the invention can be practiced without these specific details.

The microtubule-associated protein tau has been strongly implicated in Alzheimer's disease (AD) and related dementias. Upon hyperphosphorylation, tau aggregates into insoluble neurofibrillary tangles (NFTs). Tau aggregation causes dysfunction of a myriad of cellular properties including aggregate-associated toxicity, an impairment of the cellular quality control machinery, and trans-synaptic spreading of aggregate template pathologic “seeds” to adjacent neurons that leads to widespread impairment of brain function. Regardless of the molecular basis for neurotoxicity of tau, accelerating, reducing or preventing tau aggregation should alleviate pathogenesis of dementia in AD and other tauopathies.

Numerous hypotheses for pathogenesis and therefore potential therapeutic targets have been posited for AD: β-amyloid (Aβ) plaques, tau aggregation, chronic inflammation, oxidative stress, and acetylcholine abnormalities. While immunotherapeutic approaches targeting tau are enticing, one of the concerns for treatment in the preclinical phase are an undesired immune response. Likewise, small molecule approaches have typically displayed poor pharmacokinetics and off-target binding side effects. Here, we propose that PLPs can be modularly designed to directly incorporate key peptide fragments implicated in the pathogenesis of AD, followed by a systematic investigation to determine the specific mechanisms that drive tau aggregation in AD.

The dynamic and unstructured peptide sidechains of PLPs described herein serve as a counterpoint to the prevailing view that an exact conformation of antibody is needed to target disordered tau, while the brush architecture offers multivalency in binding not seen in small molecules, which mimics the multivalency of disordered proteins. Further, the disclosed PLPs are inherently metaphilic in nature (hydrophobic backbones with hydrophilic side chains, combined with globular but fluxional structures). In embodiments, this is a feature that is exploited for cell penetration and for driving the materials into phase-separated states.

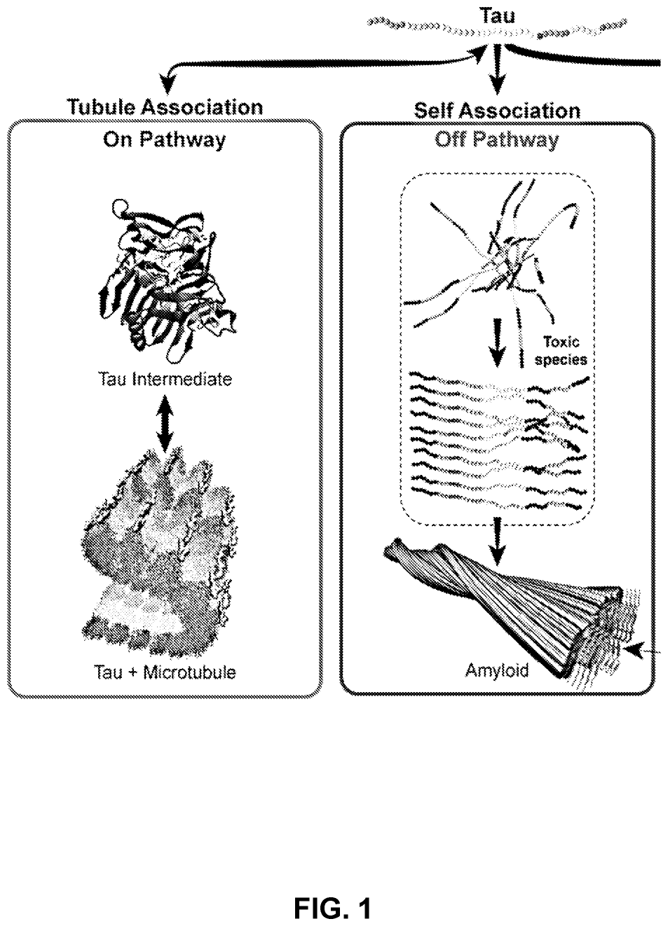

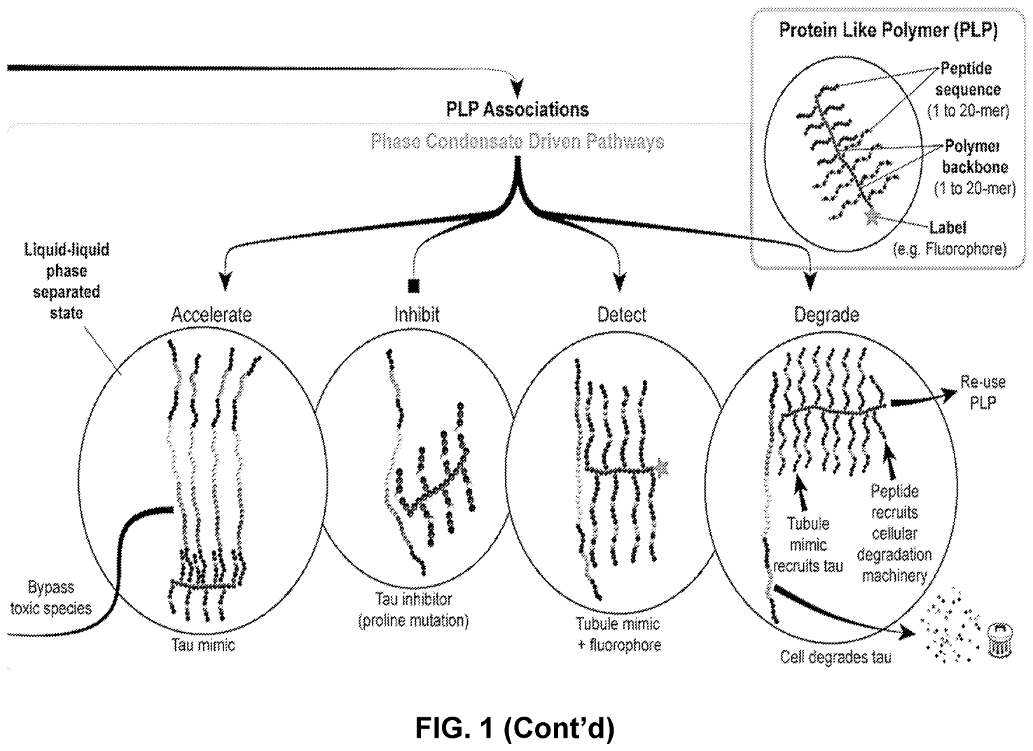

It is believed that the PLPs described herein are ideal disordered protein-targeting therapeutics by possessing one or more, or preferably all, of the following properties: 1) antibody-like specificity, while exhibiting conformation agnostic binding to the target protein, 2) the ability to alter the phase separation properties of the target protein, and 3) a sufficient interaction energy with the target protein to induce a change in the energy landscape in the context of the cell by speeding aggregation, capping aggregation, dissolving condensates, or degrading aggregates (FIG. 1). In some aspects of the invention, a polymer is disclosed that is capable of assembly into phase separated states of multiple types. In some aspects, the polymer's ability to bind with a target is not conformation dependent. In keeping with this aspect, it is believed this non-conformation dependent characteristic facilitates binding to non-aggregated tau protein.

In some embodiments, the invention provides a polymer comprising a first polymer segment comprising at least 2 first repeating units; wherein each of the first repeating units of the first polymer s comprises a first polymer backbone subunit directly or indirectly covalently linked to a first functional side chain group comprising a peptide; which (i) inhibits aggregation of, (ii) accelerates aggregation of, (iii) binds to, and/or (iv) mimics at least a portion of tau protein. In some embodiments, the invention provides a polymer comprising a first polymer segment comprising at least 2 first repeating units; wherein each of the first repeating units of the first polymer s comprises a first polymer backbone subunit directly or indirectly covalently linked to a first functional side chain group comprising a peptide; which (i) inhibits aggregation of, (ii) accelerates aggregation of, (iii) binds to, and/or (iv) mimics at least a portion of microtubulin protein. In some embodiments, the invention provides a polymer comprising a first polymer segment comprising at least 2 first repeating units; wherein each of the first repeating units of the first polymer s comprises a first polymer backbone subunit directly or indirectly covalently linked to a first functional side chain group comprising a peptide; wherein the peptide comprises a sequence having 75% or greater (e.g., 80% or greater, 85% or greater, 90% or greater, 95% or greater, or 100%) sequence identity of one or more of SEQ ID NO: 1-16. The inventive polymer can be any suitable polymer type described herein and can comprise, or be derived from, any suitable number of monomers. For example, in some embodiments, the polymer is a homopolymer (i.e., derived from/incorporating one type of monomer). Alternatively, in some embodiments, the polymer can be a copolymer comprising (e.g., derived from/incorporating) more than one type of monomer (e.g., from 2 to 10 types of monomers). It will be understood that the inventive polymer, along with the linked polymer side chains, can have any suitable configuration. For example, in some embodiments wherein the polymer is a homopolymer, the polymer can be a brush polymer. In other embodiments wherein the polymer is a copolymer, the polymer can be a brush block copolymer or brush random/statistical copolymer.

In some aspects, the polymer is characterized by a P¹ peptide density selected from the range 50% to 100%, optionally some embodiments a density selected from the range of 75% to 100%, or optionally for some embodiments a density selected from the range of 90% to 100%. In aspects, the polymer is characterized by a P² peptide density selected from the range 50% to 100%, optionally some embodiments a density selected from the range of 75% to 100%, or optionally for some embodiments a density selected from the range of 90% to 100%.

In aspects, the peptide of the functional side chain group comprises any suitable number of amino acid units so long as the peptide comprises a sequence having 75% or greater (e.g., 80% or greater, 85% or greater, 90% or greater, 95% or greater, or 100%) sequence identity to a sequence found in a tau protein or microtubulin protein. In aspects, the peptide of the functional side chain group comprises any suitable number of amino acid units so long as the peptide comprises a sequence having 75% or greater (e.g., 80% or greater, 85% or greater, 90% or greater, 95% or greater, or 100%) sequence identity to one or more of SEQ ID: 1-5 or SEQ ID: 13. In aspects, the peptide of the functional side chain group comprises any suitable number of amino acid units so long as the peptide comprises a sequence having 75% or greater (e.g., 80% or greater, 85% or greater, 90% or greater, 95% or greater, or 100%) sequence identity to one or more of SEQ ID: 6-7 or SEQ ID: 16. In aspects, the peptide of the functional side chain group comprises any suitable number of amino acid units so long as the peptide comprises a sequence having 75% or greater (e.g., 80% or greater, 85% or greater, 90% or greater, 95% or greater, or 100%) sequence identity to one or more of SEQ ID: 8-10 or SEQ ID: 14-15. In aspects, the peptide of the functional side chain group comprises any suitable number of amino acid units so long as the peptide comprises a sequence having 75% or greater (e.g., 80% or greater, 85% or greater, 90% or greater, 95% or greater, or 100%) sequence identity to one or more of SEQ ID: 11-12. In some aspects, the peptide comprises at least 5 amino acid residues. For example, the peptide comprises 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, or 33 or more amino acid units. Alternatively, or in addition, the peptide can comprise 100 or less amino acid units, for example, 90 or less, 80 or less, 70 or less, 60 or less, 59 or less, 58 or less, 57 or less, 56 or less, 55 or less, 54 or less, 53 or less, 52 or less, 51 or less, 50 or less, 49 or less, 48 or less, 47 or less, 46 or less, 45 or less, 44 or less, 43 or less, 42 or less, 41 or less, 40 or less amino acid units. Thus, the peptide can comprise a number of amino acid units bounded by any two of the aforementioned endpoints. For example, the peptide can comprise 5 to 100 amino acid units, for example, 5 to 100, 5 to 90, 5 to 80, 5 to 70, 5 to 60, 5 to 50, 5 to 40, 5 to 30, 5 to 20, 5 to 16, 5 to 15, 5 to 14, 6 to 100, 6 to 90, 6 to 80, 6 to 70, 6 to 60, 6 to 50, 6 to 40, 6 to 30, 6 to 20, 6 to 16, 6 to 15, 6 to 14, 7 to 100, 7 to 90, 7 to 80, 7 to 70, 7 to 60, 7 to 50, 7 to 40, 7 to 30, 7 to 20, 7 to 16, 7 to 15, 7 to 14, 8 to 100, 10 to 100, 10 to 90, 10 to 80, 10 to 70, 10 to 60, 10 to 50, 10 to 40, 10 to 30, 10 to 20, 10 to 16, 10 to 15, 10 to 14, 11 to 16, 11 to 15, 11 to 14, 12 to 20, 12 to 16, 12 to 15, or 12 to 14 amino acid units. In some embodiments, the peptide comprises 6 to 20 amino acid residues. In certain embodiments, the peptide comprises 6 to 16 amino acid residues.

The functional side group comprising a peptide can have any suitable structure (e.g., primary, secondary, tertiary, or quaternary structure) described herein. The peptide can be a branched peptide, a linear peptide, cyclic peptide, or a cross-linked peptide. In some embodiments, the polymer is characterized by a structure wherein at least a portion of the peptide is linked to the polymer backbone group via an enzymatically degradable linker, such a matrix metalloproteinase (MMP) cleavage sequence, cathepsin B cleavage sequence, ester bond, reductive sensitive bond-disulfide bond, pH sensitive bond-imine bond or any combinations of these. In other embodiments, the polymer is characterized by a structure wherein at least a portion of the peptide side-chain is linked to the polymer backbone or consists of a degradable or triggerable linker.

In some aspects, the peptide comprises a sequence having hydrophobic regions, such as leucine-rich regions. In aspects, the hydrophobic regions may be modified to substitute hydrophilic amino acid residues or non-hydrophobic amino acid residues. It is believed that such substitutions may facilitate improved solubility of the polymer if necessary for certain applications. In aspects where superior stability is desired, the polymer may also be modified. Acceptable polymer modifications include asparagine O-hydroxylation, higher degrees of polymerization (e.g., greater than 10 DP, greater than 15 DP, greater than 30 DP, or greater than 45 DP), single point mutations, and other suitable modifications.

Additionally, the peptide may comprise one or more gaps in its sequence. For example, the peptide may comprise 5 consecutive amino acid residues which do not impact, or do not contribute to, the properties of the peptide's sequence. In aspects, the one or more gaps is a spacer molecule, 5 or less amino acid residues, 3 or less amino acid residues, or a combination thereof.

In some specific embodiments, the polymer comprises a tag for imaging and/or analysis. In aspects, the polymer comprises a fluorescein-, biotin-, or rhodamine-based tag resulting in a fluorescently labelled PLP. For example, each polymer segment B¹, B², or B³ of formula (FX1) can independently comprise a tag for imaging and/or analysis. Similarly, each P¹ or P² of formula (FX1) can independently comprise a tag for imaging and/or analysis. Additionally, each T¹ or T² of any of the formulas described herein can independently comprise a tag for imaging and/or analysis. For example, the polymer can comprise one or more of a dye, a radiolabeling agent, an imaging agent, titration agent, and the like.

The inventive polymers can have any suitable degrees of polymerization. If the degree of polymerization is too low, the polymer may not be resistant to adhesive force or may not be resistant to enzymatic cleavage by proteases or may be cleared too rapidly from the body since the polymer's molecular weight would be lower than the clearance threshold through the kidney. Additionally, if too low, the polymer may exhibit poor solubility and structural instability. Alternatively, if the degree of polymerization is too high, the peptide side chain groups displayed on the polymer may be too dense to engage their biological targets such as cell receptors, enzymes, PPIs etc. Additionally, the high degree of polymerization may result in a polymer that is too large to penetrate cells. Typically, the polymer has a degree of polymerization of 2 to 1000 (e.g., 2 to 500, 2 to 250, 2 to 100, 2 to 60, 2 to 50, 2 to 30, 5 to 1000, 5 to 500, 5 to 250, 5 to 100, 5 to 60, 5 to 50, 5 to 45, 5 to 30, 7 to 45, 20 to 500, 20 to 250, 20 to 100, 20 to 50, or 20 to 30). In certain embodiments, the polymer has a degree of polymerization of 5 to 100. In preferred embodiments, the polymer has a degree of polymerization of 7 to 30. For example, the polymer can have a degree of polymerization of 2 or about 2, 5 or about 5, a degree of polymerization of 10 or about 10 (e.g., 11), a degree of polymerization of 15 or about 15 (e.g., 17), a degree of polymerization of 20 or about 20, a degree of polymerization of 30 or about 30, a degree of polymerization of 50 or about 50, a degree of polymerization of 60 or about 60, a degree of polymerization of 100 or about 100, a degree of polymerization of 150 or about 150, or a degree of polymerization of 200 or about 200. In some embodiments, the polymer has a degree of polymerization of 2 to 50. In certain embodiments, the polymer has a degree of polymerization of at least 5. In other certain embodiments, the polymer has a degree of polymerization of at least 7.

Additionally, in aspects the polymer comprises a brush density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects have a brush density selected from the range 50% to 100%, optionally some embodiments a density selected from the range of 75% to 100%, or optionally for some embodiments a density selected from the range of 90% to 100%. Brush polymers of preferred aspects have a brush density selected from the range 75% to 100%. Brush polymers of certain aspects have a “high brush density” selected from the range 90% to 100%, optionally some embodiments a density selected from the range of 95% to 100%, or optionally for some embodiments a density selected from the range of 99% to 100%. For example, in aspects of the invention, the polymer may be characterized by a formulation wherein 90% of its polymer segments comprise a polymer backbone group covalently linked to a functional side chain group, wherein each polymer segment may comprise the same or different functional side chain group. In aspects, said functional side chain group comprises a peptide which comprises a sequence having 75% or greater sequence identity of a sequence found in a tau protein, such as a portion of K18 tau. In aspects, said functional side chain group comprises a peptide which comprises a sequence having 75% or greater sequence identity of a sequence found in a microtubulin protein, such as SEQ ID: 6. In some embodiments, the brush density of the polymer is equal to the peptide density of a particular peptide (e.g., all polymer segments of the polymer comprising polymer backbones covalently linked to a polymer side chain comprising P¹). In other aspects, the brush density of the polymer is different from the peptide density of a particular peptide (e.g., at least one polymer segment comprises a polymer backbone covalently linked to a polymer side chain comprising P¹ and at least one other polymer segment comprises a polymer backbone covalently linked to a polymer side chain comprising P² wherein P¹ and P² are characterized by different sequences).

Additionally, for each of the polymers characterized by the formula (FX1) described herein, it will be understood that the first repeating unit

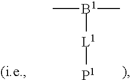

the second repeating unit

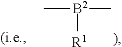

when present, and the third repeating unit

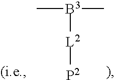

when present, can be arranged in any suitable order. For example, the first repeating unit, the second repeating unit, and the third repeating unit can be arranged as a random polymer, block polymer, brush, brush block, alternating, segmented, grafted, tapered and other architectures. In other words, variables “m”, “n”, and “o” merely define the total number of that particular monomer in the polymer and do not imply any particular order. For each of the polymers characterized by the formula (FX1), each of m, n, and o can be independently selected from any suitable integer. Suitability of the integer is based on the desired DP for each repeating unit as discussed herein.

The polymer backbone groups units (e.g., each of B¹, B², and B³ of the polymers characterized by the formula (FX1) and/or the substructures (S1a)-(S2c)), can be independently selected from any suitable polymer backbone subunit. In aspects, each of the first polymer backbone subunit, the second polymer backbone subunit, and the third polymer backbone subunit can be a monomer capable of undergoing ring opening metathesis. For example, each of B¹, B², and B³ can independently be a substituted or unsubstituted norbornene, oxanorbornene, olefin, cyclic olefin, cyclooctene, or cyclopentadiene. In some aspects, each of the first polymer backbone group subunit, the second polymer backbone group subunit, and/or the third polymer backbone group subunit is a polymerized norbornene dicarboxyimide monomer. In some embodiments, each polymer backbone subunit of the polymer is a polymerized norbornene dicarboxyimide monomer. In aspects where the polymer has poor solubility, one or more of the polymer backbone subunits may be substituted with an oxanorbornene-based subunit (if not already in use) or other suitable hydrophilic backbone subunit.

Preferably, in any embodiment of a polymer, a method, a use, a composition, or a medicament disclosed herein, the polymer is stable against enzymatic digestion. Optionally in any embodiment of a a polymer, a method, a use, a composition, or a medicament disclosed herein, the polymer is stable against enzymatic digestion by a metalloproteinase. Optionally in any embodiment of a polymer, a method, a use, a composition, or a medicament disclosed herein, the polymer is stable against enzymatic digestion by matrix metalloproteinases and thermolysin. Preferably in any embodiment of a polymer, a method, a use, a composition, or a medicament disclosed herein, the polymer is stable against enzymatic digestion for at least 450 minutes. Optionally in any embodiment a polymer, a method, a use, a composition, or a medicament disclosed herein, each polymer individually solvated by water when a plurality of said polymers is dispersed in water.

In another aspect, the invention provides a pharmaceutical composition comprising one or more peptides and/or one or more polymers described herein. In some embodiments, the composition comprises one or more pharmaceutically acceptable excipients. For example, the peptides and/or polymers of the invention can be formulated for parenteral administration, such as intravenous (IV) administration or administration into a body cavity or lumen of an organ. Alternatively, the peptides and/or polymers can be injected intra-tumorally. Formulations for injection will commonly comprise a solution of the peptide and/or polymer dissolved in a pharmaceutically acceptable carrier. Among the acceptable vehicles and solvents that can be employed are water and an isotonic sodium chloride. In addition, sterile fixed oils can conventionally be employed as a solvent or suspending medium. For this purpose, any bland fixed oil can be employed including synthetic monoglycerides or diglycerides. In addition, fatty acids such as oleic acid can likewise be used in the preparation of injectables. These solutions are sterile and generally free of undesirable matter. These formulations can be sterilized by conventional, well known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the peptide and/or polymer in these formulations can vary widely, and will be selected primarily based on fluid volumes, viscosities, body weight, and the like, in accordance with the particular mode of administration selected and the patient's needs. In certain embodiments, the concentration of a peptide and/or polymer in a solution formulation for injection will range from 0.1% (w/w) to 10% (w/w) or about 0.1% (w/w) to about 10% (w/w).

The present invention further provides a method for making the inventive polymers disclosed herein. In aspects of the method for making the inventive polymer, the at least one peptide is capped at the terminal end with any suitable polymerizable monomer. In aspects, the polymerizable monomer may comprise an ethylenically unsaturated monomer. In aspects, the polymerizable monomer may comprise an olefin-based functional group, a norbornene-amide hexanoic acid, (meth)acrylate, or a norbornene dicarboxyamide. The polymerizable monomers may be polymerized by ROMP, RAFT, or ATRP, aspects of which are further described in Kammeyer et al., Polymerization of Protecting-Group-Free Peptides via ROMP, Polym. Chem. 2013, 4 (14), 3929-3933 and Nomura et al., Precise Synthesis of Polymers Containing Functional End Groups by Living Ring-Opening Metathesis Polymerization (ROMP): Efficient Tools for Synthesis of Block Graft Copolymers, Polym. 2010, 51(9), 1861-1881, each of which is incorporated by reference herein in its entirety, and more specifically for methodologies of making a polymer, to the extent not inconsistent with the description herein.

After polymerization the inventive polymers may be characterized using any suitable technique(s). Typically, the inventive polymers are characterized by SDS-PAGE or size-exclusion chromatography with multiangle light scattering (SEC-MALS), sometimes referred to as gel permeation chromatography (GPC), to ascertain degree of polymerization (DP) and molecular weight distribution (dispersity or Mw/Mn). Alternatively, or in addition to, the inventive polymers may be characterized by SDS-PAGE to ascertain degree of polymerization (DP) and molecular weight. Preferably, there is suitable agreement between the obtained DP and the theoretical DP based on the initial monomer-to-initiator ratio ([M]₀/[I]₀).

The present invention further provides methods for using the inventive polymers disclosed herein. In aspects, the inventive polymers may be used as a therapeutic, a PPI disrupting agent, a tau or microtubulin aggregation detecting agent, a tau or microtubulin aggregation accelerating agent, or any combination thereof. In some embodiments, the methods described herein can be used to treat or manage a neurodegenerative disease or condition, such as Alzheimer's disease (AD). In some embodiments, the methods described herein can be used to treat or manage a tauopathy-related disease or condition. The method includes administering a therapeutically effective amount of a polymer described herein and a pharmaceutically acceptable excipient to a subject, a cell, or a tissue in need thereof. In some aspects, the proper dosage of the pharmaceutical composition is determined in a conventional manner, based upon factors such as the subject's condition, immune status, body weight and age. For example, the amount of polymer or peptide required to be administered for treating or managing a neurodegenerative disease or condition in a subject will vary depending upon factors such as the risk and severity of the underlying condition(s), any other medical conditions or diseases, age, the form of the composition, and other medications being administered. Further the amount may vary depending upon whether the polymer or peptide is being used to treat (when the dose may be higher) or whether the polymer or peptide is being used as a secondary prevention/maintenance (when the dose may be lower). However, the required amount can be readily set by a medical practitioner. For example, the methods can include administering the polymer to provide a dose of from 10 ng/kg to 50 mg/kg to the subject. For example, the polymer dose can range from 5 mg/kg to 50 mg/kg, from 10 μg/kg to 5 mg/kg, or from 100 μg/kg to 1 mg/kg. The polymer dose can also lie outside of these ranges, depending on the particular polymer as well as the type of disease being treated. Frequency of administration can range from a single dose to multiple doses per week, or more frequently. In some embodiments, the polymer is administered from about once per month to about five times per week. In some embodiments, the polymer is administered once per week.

The polymer can be administered by oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. In some embodiments, the polymer is administered intravenously, subcutaneously, intramuscularly, topically, orally, or a combination thereof. In some embodiments, the polymer is administered to a subject's brain, spinal cord, cerebrospinal fluid, or any combination thereof. The methods described herein can comprise contacting a target tissue of the subject with the polymer or a metabolite or product thereof, contacting a target cell of the subject with the polymer or a metabolite or product thereof, and/or contacting a target receptor of the subject with the polymer or a metabolite or product thereof, and/or contacting a target peptide of the subject with the polymer or a metabolite or product thereof. In embodiments, the polymers described herein pass through the cell membrane and contact an intracellular target. Without wishing to be bound by any particular theory, it is believed that the PLP structure and charge described herein play an integral role in providing cell permeability.

### Aspects of the Invention

Various aspects are contemplated herein, several of which are set forth in the paragraphs below. It is explicitly contemplated that any aspect or portion thereof can be combined to form an aspect. Furthermore, although the aspects below are subdivided into aspects A, B, C, D, and so forth, it is explicitly contemplated that aspects in each of subdivisions A, B, C, D, etc. can be combined in any manner. Moreover, the term “any preceding aspect” means any aspect that appears prior to the aspect that contains such phrase (in other words, the sentence “Aspect B13: The method of any one of aspects B1—B12, or any preceding aspect, . . . ” means that any aspect prior to aspect B13 is referenced, including aspects B1-B12 and all of the “A” aspects). For example, it is contemplated that, optionally, any method or composition of any of the below aspects may be useful with or combined with any other aspect provided below. Further, for example, it is contemplated that any embodiment described elsewhere herein, including above this paragraph, may optionally be combined with any of the below listed aspects. In some instances in the aspects below, or elsewhere herein, two open ended ranges are disclosed to be combinable into a range. For example, “at least X” is disclosed to be combinable with “less than Y” to form a range, in which X and Y are numeric values. For the purposes of forming ranges herein, it is explicitly contemplated that “at least X” combined with “less than Y” forms a range of X-Y inclusive of value X and value Y, even through “less than Y” in isolation does not include Y.

Aspect A1: A polymer comprising a first repeating unit comprising a first polymer backbone subunit directly or indirectly covalently linked to a first functional sidechain comprising a peptide, which (i) inhibits aggregation of, (ii) accelerates aggregation of, (iii) binds to, and/or (iv) mimics at least a portion of tau protein.

Aspect B1: A polymer comprising a first repeating unit comprising a first polymer backbone subunit directly or indirectly covalently linked to a first functional sidechain comprising a peptide, which (i) inhibits aggregation of, (ii) accelerates aggregation of, (iii) binds to, and/or (iv) mimics at least a portion of microtubulin protein.

Aspect C1: A polymer characterized by a formula (FX1):

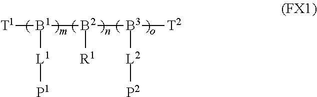

wherein:
each P¹ independently comprises a peptide;
each P² independently comprises a peptide, and each instance of P² is different from each instance of P¹;
at least one P¹ independently, or in combination with other instances of P¹, (i) inhibits aggregation of, (ii) accelerates aggregation of, (iii) binds to, and/or (iv) mimics:
(a) at least a portion of tau protein, and/or
(b) at least a portion of microtubulin protein;
T¹ and T² are each independently polymer backbone terminating groups that can be the same or different;
B¹, B², and B³ are each independently a polymer backbone subunit;
L¹ and L² are each independently a linking group;
R¹ is independently a substituent;
m is an integer from 2 to 1000 (e.g., 2 to 1000, 2 to 500, 2 to 100, 2 to 50, 2 to 30, 5 to 1000, 5 to 500, 5 to 250, 5 to 100, 5 to 50, 5 to 30, 7 to 1000, 7 to 500, 7 to 250, 7 to 100, 7 to 50, 7 to 30, 20 to 500, 20 to 250, 20 to 100, 20 to 50, 20 to 30);
n is an integer from 0 to 1000 (e.g., 0 to 500, 0 to 250, 0 to 100, 0 to 50, 0 to 30, 2 to 1000, 2 to500, 2 to250, 2 to 100, 2 to50, 2 to30, 5 to 1000, 5 to500, 5 to250, 5 to 100, 5 to50, 5 to30, 7 to 1000, 7 to 500, 7 to 250, 7 to 100, 7 to 50, 7 to 30, 20 to 500, 20 to 250, 20 to 100, 20 to 50, or 20 to 30);
is an integer from 0 to 1000 (e.g., 0 to 500, 0 to 250, 0 to 100, 0 to 50, 0 to 30, 2 to 1000, 2 to500, 2 to250, 2 to 100, 2 to50, 2 to30, 5 to 1000, 5 to500, 5 to250, 5 to 100, 5 to50, 5 to30, 7 to 1000, 7 to 500, 7 to 250, 7 to 100, 7 to 50, 7 to 30, 20 to 500, 20 to 250, 20 to 100, 20 to 50, or 20 to 30);
each connecting line in the formula (FX1) represents a covalent linkage comprising at least one of a single bond, a double bond, one or more atoms, or any combination thereof, optionally wherein the one or more atoms comprise carbon, nitrogen, and/or oxygen atoms;
each instance of B¹, B², B³, L¹, L², R¹, P¹, and P² is the same as or different from any other instance of B¹, B², B³, L¹, L², R¹, P¹, and P², respectively; and
when (i) n is an integer from 1 to 1000, o is an integer from 1 to 1000, at least one instance of P¹ is different from another instance of P¹, and/or at least one instance of P² is different from another instance of P², then (ii) the polymer is a block copolymer or a statistical copolymer.

Aspect C2: The polymer of aspect C1, or any preceding aspect, wherein:
each instance of P¹ independently comprises from 5 to 50 amino acids (e.g., 5 to 50, 5 to 40, 5 to 30, 5 to 20, 6 to 50, 6 to 40, 6 to 30, or 6 to 20 amino acids).

Aspect C3: The polymer of aspect C1 or C2, or any preceding aspect, wherein:
at least one P¹ comprises a sequence having 75% or greater (e.g., 75% or greater, 80% or greater, 85% or greater, 90% or greater, 95% or greater, or 99% or greater) sequence identity of:

SEQ ID NO: 1
(VQIVYK);
SEQ ID NO: 2
(DRVQIVYKRR);
SEQ ID NO: 3
(VQIINK);
SEQ ID NO: 4
(VQIINKRR);
SEQ ID NO: 5
(KVQIINKKLDRR);
SEQ ID NO: 6
(YQQYQDATADEQG);
SEQ ID NO: 7
(YQQYQDATADEQGRRR);
SEQ ID NO: 8
(WMINK);
SEQ ID NO: 9
(WMINKRR);
SEQ ID NO: 10
(VQPINK);
SEQ ID NO: 13
(VQIVYKRR);
SEQ ID NO: 14
(DRWMINKRR);
SEQ ID NO: 15
(VQPINKR);
or
SEQ ID NO: 16
(YQQYQDATADEQGRR).

Aspect C4: The polymer of any one of aspects C₁-C₃, or any preceding aspect, wherein:
at least one P¹ comprises a sequence having 85% (e.g., 85% or greater, 90% or greater, 95% or greater, or 99% or greater) or greater sequence identity of:

SEQ ID NO: 1
(VQIVYK);
SEQ ID NO: 2
(DRVQIVYKRR);
SEQ ID NO: 3
(VQIINK);
SEQ ID NO: 4
(VQIINKRR);
SEQ ID NO: 5
(KVQIINKKLDRR);
SEQ ID NO: 6
(YQQYQDATADEQG);
SEQ ID NO: 7
(YQQYQDATADEQGRRR);
SEQ ID NO: 8
(WMINK);
SEQ ID NO: 9
(WMINKRR);
SEQ ID NO: 10
(VQPINK);
SEQ ID NO: 13
(VQIVYKRR);
SEQ ID NO: 14
(DRWMINKRR);
SEQ ID NO: 15
(VQPINKR);
or
SEQ ID NO: 16
(YQQYQDATADEQGRR).

Aspect C5: The polymer of any one of aspect C₁-C₄, or any preceding aspect, wherein:
at least one P¹ comprises:

SEQ ID NO: 1
(VQIVYK);
SEQ ID NO: 2
(DRVQIVYKRR);
SEQ ID NO: 3
(VQIINK);
SEQ ID NO: 4
(VQIINKRR);
SEQ ID NO: 5
(KVQIINKKLDRR);
SEQ ID NO: 6
(YQQYQDATADEQG);
SEQ ID NO: 7
(YQQYQDATADEQGRRR);
SEQ ID NO: 8
(WMINK);
SEQ ID NO: 9
(WMINKRR);
SEQ ID NO: 10
(VQPINK);
SEQ ID NO: 13
(VQIVYKRR);
SEQ ID NO: 14
(DRWMINKRR);
SEQ ID NO: 15
(VQPINKR);
or
SEQ ID NO: 16
(YQQYQDATADEQGRR).

Aspect C6: The polymer of any one of aspects C₁-C₅, or any preceding aspect, wherein:
at least a portion of each instance of P¹ independently comprises:

SEQ ID NO: 1
(VQIVYK);
SEQ ID NO: 2
(DRVQIVYKRR);
SEQ ID NO: 3
(VQIINK);
SEQ ID NO: 4
(VQIINKRR);
SEQ ID NO: 5
(KVQIINKKLDRR);
SEQ ID NO: 6
(YQQYQDATADEQG);
SEQ ID NO: 7
(YQQYQDATADEQGRRR);
SEQ ID NO: 8
(WMINK);
SEQ ID NO: 9
(WMINKRR);
SEQ ID NO: 10
(VQPINK);
SEQ ID NO: 13
(VQIVYKRR);
SEQ ID NO: 14
(DRWMINKRR);
SEQ ID NO: 15
(VQPINKR);
SEQ ID NO: 16
(YQQYQDATADEQGRR);

any combination thereof.

Aspect C7: The polymer of any one of aspects C₁-C₆, or any preceding aspect, wherein:
at least 75% (e.g., 75% or greater, 80% or greater, 85% or greater, 90% or greater, 95% or greater, or 99% or greater) of all instances of P¹ on a number basis independently comprise:

SEQ ID NO: 1
(VQIVYK);
SEQ ID NO: 2
(DRVQIVYKRR);
SEQ ID NO: 3
(VQIINK);
SEQ ID NO: 4
(VQIINKRR);
SEQ ID NO: 5
(KVQIINKKLDRR);
SEQ ID NO: 6
(YQQYQDATADEQG);
SEQ ID NO: 7
(YQQYQDATADEQGRRR);
SEQ ID NO: 8
(WMINK);
SEQ ID NO: 9
(WMINKRR);
SEQ ID NO: 10
(VQPINK);
SEQ ID NO: 13
(VQIVYKRR);
SEQ ID NO: 14
(DRWMINKRR);
SEQ ID NO: 15
(VQPINKR);
SEQ ID NO: 16
(YQQYQDATADEQGRR);

or
any combination thereof.

Aspect C8: The polymer of any one of aspects C1-C7, or any preceding aspect, wherein:
at least one P¹ comprises a sequence having 75% or greater (e.g., 75% or greater, 80% or greater, 85% or greater, 90% or greater, 95% or greater, or 99% or greater) sequence identity of:

SEQ ID NO: 2
(DRVQIVYKRR);
SEQ ID NO: 4
(VQIINKRR);
SEQ ID NO: 10
(VQPINK);
or
SEQ ID NO: 15
(VQPINKR).

Aspect C9: The polymer of any one of aspects C1-C8, or any preceding aspect, wherein:
at least one P¹ comprises a sequence having 85% or greater (e.g., 85% or greater, 90% or greater, 95% or greater, or 99% or greater) sequence identity of:

SEQ ID NO: 2
(DRVQIVYKRR);
SEQ ID NO: 4
(VQIINKRR);
SEQ ID NO: 10
(VQPINK);
or
SEQ ID NO: 15
(VQPINKR).

Aspect C10: The polymer of any one of aspects C1-C9, or any preceding aspect, wherein:
at least one P¹ comprises:

SEQ ID NO: 2
(DRVQIVYKRR);
SEQ ID NO: 4
(VQIINKRR);
SEQ ID NO: 10
(VQPINK);
or
SEQ ID NO: 15
(VQPINKR).

Aspect C11: The polymer of any one of aspects C1-C10, or any preceding aspect, wherein:
at least a portion of each instance of P independently comprises:

SEQ ID NO: 2
(DRVQIVYKRR);
SEQ ID NO: 4
(VQIINKRR);
SEQ ID NO: 10
(VQPINK);
or
SEQ ID NO: 15
(VQPINKR).

or
any combination thereof.

Aspect C12: The polymer of any one of aspects C1-C11, or any preceding aspect, wherein:
at least 75% (e.g., 75% or greater, 80% or greater, 85% or greater, 90% or greater, 95% or greater, or 99% or greater) of all instances of P¹ on a number basis comprise:

SEQ ID NO: 2
(DRVQIVYKRR);
SEQ ID NO: 4
(VQIINKRR);
SEQ ID NO: 10
(VQPINK);
or
SEQ ID NO: 15
(VQPINKR).

or
any combination thereof

Aspect C13: The polymer of any one of aspects C1-C12, or any preceding aspect, wherein:
at least one P¹ comprises:

SEQ ID NO: 1
(VQIVYK)
or
SEQ ID NO: 2
(DRVQIVYKRR);

and
at least one other P¹ comprises:

SEQ ID NO: 8
(WMINK)
or
SEQ ID NO: 9
(WMINKRR).

Aspect C14: The polymer of aspect C13, or any preceding aspect, wherein the polymer is characterized by proportionate amounts of the at least one P¹ and the at least one other P¹.

Aspect C15: The polymer of aspect C13, or any preceding aspect, wherein the polymer is characterized by a peptide ratio of the at least one P¹: the at least one other P¹ of 1:14. For example, a peptide ratio between the range of 0.8:14 to 1.2:14 or 1:11.2 to 1:16.8.

Aspect C16: The polymer of aspect C14, or any preceding aspect, wherein the polymer is characterized by a peptide ratio of the at least one P¹: the at least one other P¹ of 7:8. For example, a peptide ratio between the range of 5.6:8 to 8.4:8 or 7:6.4 to 7:9.6.

Aspect C17: The polymer of aspect C14, or any preceding aspect, wherein the polymer is characterized by a peptide ratio of the at least one P¹: the at least one other P¹ of 14:1. For example, a peptide ratio between the range of 11.2:1 to 16.8:1 or 14:0.8 to 14:1.2.

Aspect C18: The polymer of any one of aspects C1-C17, or any preceding aspect, wherein:
at least one P¹ comprises:

SEQ ID NO: 4
(VQIINKRR)
or
SEQ ID NO: 3
(VQIINK);

and
at least one other P¹ comprises:

SEQ ID NO: 8
(WMINK)
or
SEQ ID NO: 9
(WMINKRR).

Aspect C19: The polymer of aspect C18, or any preceding aspect, wherein the polymer is characterized by proportionate amounts of the at least one P¹ and the at least one other P¹.

Aspect C20: The polymer of aspect C18, or any preceding aspect, wherein the polymer is characterized by a peptide ratio of the at least one P¹: the at least one other P¹ of 1:14. For example, a peptide ratio between the range of 0.8:14 to 1.2:14 or 1:11.2 to 1:16.8.

Aspect C21: The polymer of aspect C18, or any preceding aspect, wherein the polymer is characterized by a peptide ratio of the at least one P¹: the at least one other P¹ of 7:8. For example, a peptide ratio between the range of 5.6:8 to 8.4:8 or 7:6.4 to 7:9.6.

Aspect C22: The polymer of aspect C18, or any preceding aspect, wherein the polymer is characterized by a peptide ratio of the at least one P¹: the at least one other P¹ of 14:1. For example, a peptide ratio between the range of 11.2:1 to 16.8:1 or 14:0.8 to 14:1.2.

Aspect C23: The polymer of any ones of aspects C1-C22, or any preceding aspect, wherein:
at least one P¹ comprises:

SEQ ID NO: 10
(VQPINK)
or
SEQ ID NO: 15
(VQPINKR);

and
at least one other P¹ comprises:

SEQ ID NO: 3
(VQIINK),
SEQ ID NO: 4
(VQIINKRR),
SEQ ID NO: 5
(KVQIINKKLDRR),
SEQ ID NO: 1
(VQIVYK),
or
SEQ ID NO: 2
(DRVQIVYKRR).

Aspect C24: The polymer of aspect C23, or any preceding aspect, wherein the polymer is characterized by proportionate amounts of the at least one P¹ and the at least one other P¹.

Aspect C25: The polymer of aspect C23, or any preceding aspect, wherein the polymer is characterized by a peptide ratio of the at least one P¹: the at least one other P¹ of 1:14. For example, a peptide ratio of 0.8:14 to 1.2:14 or 1:11.2 to 1:16.8.

Aspect C26: The polymer of aspect C23, or any preceding aspect, wherein the polymer is characterized by a peptide ratio of the at least one P¹: the at least one other P¹ of 8:7. For example, a peptide ratio between the range of 6.4:7 to 9.6:7 or 8:5.6 to 8:8.4.

Aspect C27: The polymer of aspect C23, or any preceding aspect, wherein the polymer is characterized by a peptide ratio of the at least one P¹: the at least one other P¹ of 14:1. For example, a peptide ratio between the range of 11.2:1 to 16.8:1 or 14:0.8 to 14:1.2.

Aspect C28: The polymer of any one of aspects C1-C27, or any preceding aspect, wherein:
each instance of P², if present, independently comprises 5 to 50 amino acids (e.g., 5 to 50, 5 to 40, 5 to 30, 5 to 20, 6 to 50, 6 to 40, 6 to 30, or 6 to 20 amino acids).

Aspect C29: The polymer of any one of aspects C1-C28, or any preceding aspect, wherein:
is an integer from 1 to 1000 (e.g., 1 to 500, 1 to 250, 1 to 100, 1 to 50, 1 to 30, 2 to 1000, 2 to500, 2 to250, 2 to 100, 2 to50, 2 to30, 5 to 1000, 5 to500, 5 to250, 5 to 100, 5 to50, 5 to30, 7 to 1000, 7 to 500, 7 to 250, 7 to 100, 7 to 50, 7 to 30, 20 to 500, 20 to 250, 20 to 100, 20 to 50, or 20 to 30); and
at least one P² comprises a sequence that is capable of triggering an organism's ubiquitination cellular machinery, optionally wherein the amino acid sequence binds to E3 ubiquitin ligase.

Aspect C30: The polymer of aspect C29, or any preceding aspect, wherein:
the sequence that is capable of triggering an organism's ubiquitination cellular machinery (i) has at least 75% (e.g., at least 75%, at least 80%, at least 85%, at least 90%, at least 95%, or at least 99%) or at least 90% (e.g., at least 90%, at least 95%, or at least 99%) amino acid composition similarity to, (ii) has at least 60% (e.g., at least 60%, at least 65%, at least 70%, at least 75%, at least 80%, at least 85%, at least 90%, at least 95%, or at least 99%) or at least 80% (e.g., at least 80%, at least 85%, at least 90%, at least 95%, or at least 99%) sequence identity to, or (iii) comprises:

SEQ ID NO: 11
(ALAPYIP)
or
SEQ ID NO: 12
(ALAPYIPRR).

Aspect C31: The polymer of aspect C30, or any preceding aspect, wherein:
at least one P¹ comprises SEQ ID NO: 6 (YQQYQDATADEQG), SEQ ID NO: 7 (YQQYQDATADEQGRRR), SEQ ID NO: 16 (YQQYQDATADEQGRR), SEQ ID NO: 3 (VQIINK), SEQ ID NO: 4 (VQIINKRR), SEQ ID NO: 5 (KVQIINKKLDRR), SEQ ID NO: 1 (VQIVYK), or SEQ ID NO: 2 (DRVQIVYKRR); and
at least one P² comprises SEQ ID NO: 11 (ALAPYIP) or SEQ ID NO: 12 (ALAPYIPRR).

Aspect C32: The polymer of any one of aspects C1-C31, or any preceding aspect, wherein o is an integer from 1 to 1000 (e.g., 1 to 500, 1 to 250, 1 to 100, 1 to 50, 1 to 30, 2 to 1000, 2 to 500, 2 to 250, 2 to 100, 2 to 50, 2 to 30, 5 to 1000, 5 to 500, 5 to 250, 5 to 100, 5 to 50, 5 to 30, 7 to 1000, 7 to 500, 7 to 250, 7 to 100, 7 to 50, 7 to 30, 20 to 500, 20 to 250, 20 to 100, 20 to 50, or 20 to 30) and the polymer is characterized by a peptide ratio of P¹:P² of 14:1. For example, a peptide ratio between the range of 11.2:1 to 16.8:1 or 14:0.8 to 14:1.2.

Aspect C33: The polymer of any one of aspects C1-C31, or any preceding aspect, wherein o is an integer from 1 to 1000 (e.g., 1 to 500, 1 to 250, 1 to 100, 1 to 50, 1 to 30, 2 to 1000, 2 to 500, 2 to 250, 2 to 100, 2 to 50, 2 to 30, 5 to 1000, 5 to 500, 5 to 250, 5 to 100, 5 to 50, 5 to 30, 7 to 1000, 7 to 500, 7 to 250, 7 to 100, 7 to 50, 7 to 30, 20 to 500, 20 to 250, 20 to 100, 20 to 50, or 20 to 30) and the polymer is characterized by a peptide ratio of P¹:P² of 13:2. For example, a peptide ratio between the range of 10.4:2 to 15.6:2 or 13:1.6 to 13:2.4.

Aspect C34: The polymer of any one of aspects C1-C31, or any preceding aspect, wherein o is an integer from 1 to 1000 (e.g., 1 to 500, 1 to 250, 1 to 100, 1 to 50, 1 to 30, 2 to 1000, 2 to 500, 2 to 250, 2 to 100, 2 to 50, 2 to 30, 5 to 1000, 5 to 500, 5 to 250, 5 to 100, 5 to 50, 5 to 30, 7 to 1000, 7 to 500, 7 to 250, 7 to 100, 7 to 50, 7 to 30, 20 to 500, 20 to 250, 20 to 100, 20 to 50, or 20 to 30) and the polymer is characterized by a peptide of P¹:P² of 5:1. For example, a peptide ratio between the range of 4:1 to 6:1 or 5:0.8 to 5:1.2.

Aspect C35: The polymer of any one of aspects C1-C31, or any preceding aspect, wherein o is an integer from 1 to 1000 (e.g., 1 to 500, 1 to 250, 1 to 100, 1 to 50, 1 to 30, 2 to 1000, 2 to 500, 2 to 250, 2 to 100, 2 to 50, 2 to 30, 5 to 1000, 5 to 500, 5 to 250, 5 to 100, 5 to 50, 5 to 30, 7 to 1000, 7 to 500, 7 to 250, 7 to 100, 7 to 50, 7 to 30, 20 to 500, 20 to 250, 20 to 100, 20 to 50, or 20 to 30) and the polymer is characterized by a peptide ratio of P¹:P² of 3:1. For example, a peptide ratio between the range of 2.4:1 to 3.6:1 or 3:0.8 to 3:1.2.

Aspect C36: The polymer of any one of aspects A1-C35, wherein:
the tau protein and/or microtubulin protein are in a form of oligomers, protofibrils, amyloid fibrils, cross-β sheet amyloid species, or any combination thereof.

Aspect C37: The polymer of any one of aspects C1-C36, or any preceding aspect, wherein:
at least one P¹ independently, or in combination with other instances of P¹, (i) inhibits aggregation of, (ii) accelerates aggregation of, (iii) binds to, and/or (iv) mimics (a) at least a portion of tau protein when the tau protein is in a liquid-liquid phase separated (LLPS) state.

Aspect C38: The polymer of aspect C37, or any preceding aspect, wherein:
the polymer disrupts and/or prevents further growth of the LLPS state of the tau protein.

Aspect C39: The polymer of any one of aspects A1-C38, wherein:
the polymer is capable of being in an LLPS state under physiological conditions.

Aspect C40: The polymer of any one of aspects A1-C39, wherein:
(a) the at least a portion of tau protein comprises an aggregative region of the tau protein,
(b) the at least a portion of microtubulin protein comprises an aggregative region of the microtubulin protein; or
(c) a combination thereof.

Aspect C41: The polymer of any one of aspects C1-C40, or any preceding aspect, wherein:
at least one P¹ and/or at least one P² is characterized by a net positive charge.

Aspect C42: The polymer of any one of aspects C1-C41, or any preceding aspect, wherein:
at least one P¹ and/or at least one P² is characterized by a net positive charge of 1 to 5 (e.g., a net positive charge of 1, 2, 3, 4, or 5).

Aspect C43: The polymer of any one of aspects C1-C42, or any preceding aspect, wherein:
at least 75% (e.g., at least 75%, at least 80%, at least 85%, at least 90%, at least 95%, or at least 99%) of all instances of P¹ on a number basis are characterized by a net positive charge.

Aspect C44: The polymer of any one of aspects C1-C43, or any preceding aspect, wherein:
at least 75% (e.g., at least 75%, at least 80%, at least 85%, at least 90%, at least 95%, or at least 99%) of all instances of P² on a number basis are characterized by a net positive charge.

Aspect C45: The polymer of any one of aspects C1-C44, or any preceding aspect, wherein:
at least one P¹ and/or at least one P² comprises or further comprises at least one arginine.

Aspect C46: The polymer of any one of aspects C1-C45, or any preceding aspect, wherein:
at least one P¹ and/or at least one P² comprises or further comprises at least one aspartic acid.

Aspect C47: The polymer of any one of aspects C1-C46, or any preceding aspect, wherein:
at least one P¹ and/or at least one P² comprises or further comprises at least one proline.

Aspect C48: The polymer of any one of aspects A1-C47, wherein:
the polymer is metaphilic.

Aspect C49: The polymer of any one of aspects A1-C48, wherein the polymer is characterized by an average degree of polymerization of 2 to 100 (e.g., 2 to 100, 2 to 80, 2 to 60, 2 to 50, 2 to 30, 2 to 20, 7 to 100, 7 to 80, 7 to 60, 7 to 40, 7 to 30, 7 to 20, 15 to 50, 15 to 40, or 15 to 30).

Aspect C50: The polymer of any one of aspects A1-C49, wherein the polymer is characterized by an average degree of polymerization of 2 to 50 (e.g., 2 to 50, 2 to 30, 2 to 20, 7 to 50, 4 to 40, 4 to 30, 4 to 20, 15 to 50, 15 to 40, or 15 to 30).

Aspect C51: The polymer of any one of aspects A1-C50, wherein the polymer is characterized by an average degree of polymerization of 2 to 30 (e.g., 2 to 30, 2 to 20, 5 to 30, 5 to 20, 7 to 30, 7 to 20, 15 to 40, or 15 to 30).

Aspect C52: The polymer of any one of aspects A1-C51, wherein the polymer is characterized by an average degree of polymerization of 7 to 30 (e.g., 7 to 30, 7 to 25, 7 to 20, 7 to 15).

Aspect C53: The polymer of any one of aspects A1-C52, wherein the polymer is characterized by a number average molecular weight of 1 kDa to 50 kDa (e.g., from 1 kDa to 50 kDa, from 1 kDa to 30 kDa, from 5 kDa to 50 kDa, from 5 kDa to 30 kDa, from 7 kDa to 50 kDa, from 7 kDa to 30 kDa, from 10 kDa to 50 kDa, or from 10 kDa to 30 kDa).

Aspect C54: The polymer of any one of aspects A1-C53, wherein the polymer is characterized by a number average molecular weight of 1 kDa to 30 kDa (e.g., from 1 kDa to 30 kDa, from 5 kDa to 30 kDa, from 7 kDa to 30 kDa, or from 10 kDa to 30 kDa).

Aspect C55: The polymer of any one of aspects A1-C54, wherein the polymer is characterized by a brush density of at least 75% (e.g., at least 75%, at least 80%, at least 85%, at least 90%, at least 95%, or at least 99%).

Aspect C56: The polymer of any one of aspects A1-C55, wherein the polymer is characterized by a brush density of at least 80% (e.g., at least 80%, at least 85%, at least 90%, at least 95%, or at least 99%).

Aspect C57: The polymer of any one of aspects C1-C56, or any preceding aspect, wherein the polymer has at least one of the following properties:
(a) P¹ comprises 5-100 amino acids (e.g., 5 to 100, 5 to 50, 5 to 40, 5 to 30, 5 to 20, 6 to 50, 6 to 100, 6 to 40, 6 to 30, or 6 to 20 amino acids);
(b) m is an integer from 2 to 100 (e.g., 2 to 100, 2 to 80, 2 to 60, 2 to 50, 2 to 30, 2 to 20, 2 to 10, 4 to 100, 4 to 80, 4 to 60, 4 to 50, 4 to 30, 4 to 20, 4 to 10, 10 to 50, 10 to 40, 10 to 30, 20 to 50, 20 to 40, or 20 to 30);
(c) n is an integer from 0 to 100 (e.g., 0 to 100, 0 to 80, 0 to 60, 0 to 50, 0 to 30, 0 to 20, 0 to 10, 4 to 100, 4 to 80, 4 to 60, 4 to 50, 4 to 30, 4 to 20, 4 to 10, 10 to 50, 10 to 40, 10 to 30, 20 to 50, 20 to 40, or 20 to 30);
(d) o is an integer from 0 to 100 (e.g., 0 to 100, 0 to 80, 0 to 60, 0 to 50, 0 to 30, 0 to 20, 0 to 10, 4 to 100, 4 to 80, 4 to 60, 4 to 50, 4 to 30, 4 to 20, 4 to 10, 10 to 50, 10 to 40, 10 to 30, 20 to 50, 20 to 40, or 20 to 30);
(d) m is an integer from 2 to 100 (e.g., 2 to 100, 2 to 80, 2 to 60, 2 to 50, 2 to 30, 2 to 20, 2 to 10, 4 to 100, 4 to 80, 4 to 60, 4 to 50, 4 to 30, 4 to 20, 4 to 10, 10 to 50, 10 to 40, 10 to 30, 20 to 50, 20 to 40, or 20 to 30), n is 0, p is 0, and at least one instance of P¹ is different from another instance of P¹;
(e) degree polymerization (m+n+o) is an integer from 2 to 200 (e.g., 2 to 200, 2 to 100, 2 to 80, 2 to 60, 2 to 50, 2 to 30, 2 to 20, 2 to 10, 4 to 200, 4 to 100, 4 to 80, 4 to 60, 4 to 50, 4 to 30, 4 to 20, 4 to 10, 10 to 200, 10 to 50, 10 to 40, 10 to 30, 20 to 50, 20 to 40, or 20 to 30), or 2 to 50 (e.g., 2 to 50, 2 to 30, 2 to 20, 2 to 10, 4 to 50, 4 to 30, 4 to 20, 4 to 10, 10 to 50, 10 to 40, 10 to 30, 20 to 50, 20 to 40, or 20 to 30);
(f) molecular weight of from 1 kDa to 1,000 kDa (e.g., from 1 kDa to 1000 kDa, from 1 kDa to 500 kDa, from 1 kDa to 100 kDa, from 1 kDa to 50 kDa, from 1 kDa to 30 kDa, from 5 kDa to 50 kDa, from 5 kDa to 30 kDa, from 7 kDa to 1000 kDa, from 7 kDa to 50 kDa, from 7 kDa to 30 kDa, from 10 kDa to 1000 kDa, from 10 kDa to 50 kDa, or from 10 kDa to 30 kDa);
(g) the polymer has a P¹ peptide density of at least 50% (e.g., at least 50%, at least 55%, at least 60%, at least 65%, at least 70%, at least 75%, at least 80%, at least 85%, at least 90%, at least 95%, or at least 99%), as defined by equation m/(m+n+o)×100;
(h) a combination thereof,
(i) any combination thereof.

Aspect C58: The polymer of any one of aspects C1-C57, or any preceding aspect, wherein m is an integer from 2 to 100 (e.g., 2 to 100, 2 to 80, 2 to 60, 2 to 50, 2 to 30, 2 to 20, 2 to 10, 4 to 100, 4 to 80, 4 to 60, 4 to 50, 4 to 30, 4 to 20, 4 to 10, 10 to 50, 10 to 40, 10 to 30, 20 to 50, 20 to 40, or 20 to 30), and the polymer binds multivalently via at least two P's to the at least a portion of tau protein and/or the at least a portion of microtubulin protein.

Aspect C59: The polymer of any one of aspects C1-C57, or any preceding aspect, wherein at least one of B¹, B², or B³ is, or all three are, a polymerized monomer comprising an unsaturated monomer.

Aspect C60: The polymer of aspect C59, or any preceding aspect, wherein the unsaturated monomer comprises an ethylenically unsaturated monomer, a norbornene monomer, or a norbornene dicarboxyimide.

Aspect C61: The polymer of any one of aspects A1-C60, wherein the polymer is prepared by a living polymerization method optionally selected from ring-opening metathesis polymerization (ROMP), reversible addition-fragmentation chain transfer polymerization (RAFT), or atom transfer radical polymerization (ATRP).

Aspect C62: The polymer of any one of aspects C1-C61, or any preceding aspect, wherein each instance of a substructure (Sla):

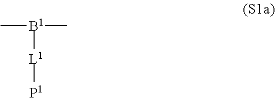

in formula (FX1) independently comprises a substructure (S1b) or a substructure (S1c):

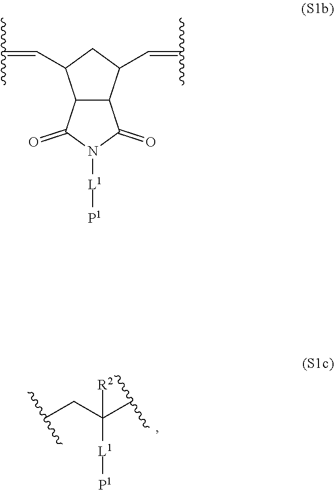

wherein R² is H or C₁-C₃ alkyl.

Aspect C63: The polymer of any one of aspects C1-C62, or any preceding aspect, wherein each instance of a substructure (S2a):

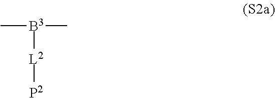

in formula (FX1) independently comprises a substructure (S2b) or a substructure (S2c):

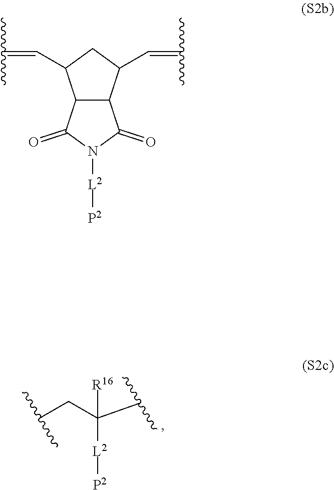

wherein R¹⁶ is H or C₁-C₃ alkyl.

Aspect C64: The polymer of any one of aspects C1-C63, or any preceding aspect, wherein each instance of L¹ and L², if present, independently is a single bond, —O—, —(CH₂CH₂O)ₓ—, C₁-C₁₀ alkyl, C₁-C₁₀ acyl, C₂-C₁₀ alkenyl, C₃-C₁₀ aryl, C₁-C₁₀ alkoxyl, or any combination thereof, wherein x is an integer from 1 to 20, and wherein each L¹ and L², if present, is configured with one or more suitable functional groups to covalently attach B¹ with P¹ and B³ with P².

Aspect C65: The polymer of any one of aspects C1-C64, or any preceding aspect, wherein each of R¹, T¹, and T² independently is hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₁₀ alkynyl, C₅-C₃₀ alkylaryl, —CO₂R³, —CONR⁴R⁵, —COR⁶, —SOR⁷, —OSR⁸, —SO₂R⁹, —OR¹⁰, —SR¹¹, —NR¹²R¹³—NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, a metal complex, a fluorophore-containing moiety, or a contrast agent-containing moiety, wherein each of R³—R¹⁵ independently is H, C₅-C₁₀ aryl, or C₁-C₁₀ alkyl.

Aspect C66: The polymer of any one of aspects C1-C65, or any preceding aspect, wherein at least one P¹, P², R¹, T¹, or T² comprises, or further comprises, a fluorophore-containing moiety or a contrast agent-containing moiety.

Aspect C67: The polymer of any one of aspects C1-C66, or any preceding aspect, wherein at least one P¹, P², R¹, T¹, or T² comprises, or further comprises, rhodamine, fluorescein, Cy5.5, gadoteric acid, or a combination thereof.

Aspect D1: A composition comprising the polymer of any of aspects A1-C67 and a pharmaceutically acceptable carrier.

Aspect E1: A medicament, for use in preventing, disrupting, accelerating, or detecting tau and/or microtubulin protein aggregation, comprising a therapeutically effective amount of a composition having the polymer of any one of aspects A1-C67.

Aspect E2: A medicament, for use in preventing, treating, or detecting a tauopathy-related disease or condition in a subject, comprising a therapeutically effective amount of a composition having the polymer of any one of aspects A1-C67.

Aspect F1: A method for preventing, disrupting, accelerating, or detecting tau and/or microtubulin protein aggregation, the method comprising:
contacting an oligomer, protofibril, amyloid fibril, and/or cross-0 sheet amyloid species of tau and/or microtubulin with a therapeutically effective amount of the polymer of any one of aspects A1-C67 or the composition of aspect D1, or any preceding aspect.

Aspect F2: The method of aspect F1, or any preceding aspect, wherein the oligomer, protofibril, amyloid fibril, and/or cross-0 sheet amyloid species of tau and/or microtubulin is in a patient or in a fluid derived from a subject.

Aspect F3: The method of aspect F1 or aspect F2, or any preceding aspect, wherein at least one R¹ is a fluorophore-containing moiety or a contrast agent-containing moiety, and the method further comprises an imaging step after the contacting step.

Aspect G1: A method for preventing, treating, or detecting a tauopathy-related disease or condition in a subject, the method comprising:
administering to the subject a therapeutically effective amount of the polymer of any one of aspects A1-C67 or the composition of aspect D1, or any preceding aspect.

Aspect G2: The method of aspect G1, or any preceding aspect, wherein the tauopathy-related disease or condition comprises a neurodegenerative disease optionally selected from or associated with Alzheimer's disease (AD), primary age-related tauopathy, chronic traumatic encephalopathy, traumatic brain injury, progressive supranuclear palsy, corticobasal degeneration, dementia, frontotemporal dementia, argyrophilic grain disease, frontotemporal dementia and parkinsonism linked to chromosome 17, Parkinson's disease, parkinsonism, postencephalitic parkinsonism, amyotrophic lateral sclerosis (ALS), Huntington's disease, vacuolar tauopathy, lytico-bodig disease, ganglioglioma, gangliocytoma, meningioangiomatosis, subacute sclerosing panencephalitis, lead encephalopathy, tuberous sclerosis, pantothenate kinase-associated neurodegeneration, lipofuscinosis, Pick's disease, Pick's complex, or any combination thereof.

Aspect G3: The method of aspect G1 or aspect G2, or any preceding aspect, wherein the polymer is administered to the subject's brain, spinal cord, cerebrospinal fluid, or any combination thereof.

Aspect G4: The method of any one of aspects G1-G3, or any preceding aspect, wherein at least one R¹ is a fluorophore-containing moiety or a contrast agent-containing moiety, and the method further comprises an imaging step after the administering step.

Aspect H1: Use of a composition for preventing, disrupting, accelerating, or detecting tau and/or microtubulin protein aggregation, wherein the composition comprises the polymer of any one of aspects A1-C67, or any preceding aspect.

Aspect I1: Use of a composition for preventing, treating, or detecting a tauopathy-related disease or condition in a subject, wherein the composition comprises the polymer of any one of aspects A1-C67, or any preceding aspect.

Aspect J1: A method of making the polymer of any one of aspects A1-C67, or any preceding aspect, the method comprising:
synthesizing the at least one P¹ peptide;
capping the at least one P¹ peptide at a terminal end with a polymerizable monomer that, once polymerized, becomes polymer backbone subunit B¹, thereby forming a polymerizable P¹ monomer;
polymerizing the polymerizable P¹ monomer.

Aspect J2: The method of aspect J1, or any preceding aspect, wherein:
the synthesizing step comprises solid-phase synthesis using protected amino acids;
the polymerizable monomer comprises an ethylenically unsaturated monomer optionally comprising norbornene or a (meth)acrylate; and
the polymerizing step comprises ROMP, RAFT, or ATRP.

Aspect K1: A method of making a medicament for use in preventing, disrupting, accelerating, or detecting Tau and/or microtubulin protein aggregation, the method comprising combining:
a therapeutically effective amount of a composition having the polymer of any one of aspects A1-C67, or any preceding aspect, and
an optional carrier.

Aspect L1: A method of making a medicament for use in preventing, treating, or detecting a tauopathy-related disease or condition in a subject, the method comprising combining:
a therapeutically effective amount of a composition having the polymer of any one of aspects A1-C67, or any preceding aspect, and
an optional carrier.

### EXAMPLES

The invention can be further understood by the following non-limiting examples. The examples are provided to illustrate some of the concepts described within this disclosure. While each example is considered to provide specific individual embodiments of composition and methods of preparation and use, none of the examples should be considered to limit the more general embodiments described herein.

In the following examples, efforts have been made to ensure accuracy with respect to numbers used (e.g., amounts, temperature, etc.) but some experimental error and deviation should be accounted for.

Materials and Methods: The following descriptions of materials and methods apply to one or more of the below Examples. To the extent of any conflict between the materials and methods of these descriptions and those provided in the Examples, the Examples control.

PLPs: The PLPs tested in the below examples are occasionally referred to as “TPX” where “X” denotes a specific peptide sequence. For example, TP1 refers to a PLP generated from peptide monomers characterized by SEQ ID: 16; TP4 refers to a PLP generated from peptide monomers characterized by SEQ ID: 4; TP7 refers to a PLP generated from peptide monomers characterized by SEQ ID: 2; and TP12 refers to a PLP generated from peptide monomers characterized by SEQ ID: 10. Similarly, free peptides (i.e., peptides which have not been incorporated into the PLP platform) in the below examples are occasionally referred to as “PX” where “X” denotes a specific peptide sequence.

Peptide Synthesis: Peptide monomers are synthesized on Rink resin (0.67 mmol/g) using standard Fmoc SPPS procedures on the Liberty Blue Automated Microwave Synthesizer. Peptide monomers are prepared via amide coupling to N-(hexanoic acid)-cis-5-norbornene-exo-dicarboximide (e.g., 3.0 equiv.) in the presence of HBTU (e.g., 2.9 equiv.) and DIPEA (e.g., 6.0 equiv.). Aspects of this process are described in detail in Blum et al., Peptides Displayed as High Density Brush Polymers Resist Proteolysis and Retain Bioactivity, J. Am. Chem. Soc. 2014, 136 (43), 15422-15437, which is incorporated by reference herein in its entirety, and more specifically for methodologies of synthesis, to the extent not inconsistent with the description herein. The peptide monomers are cleaved off the resin by treating the resin with trifluoroacetic acid (TFA):H₂O:triisopropyl silane (95:2.5:2.5 v/v) for 4 h. The crude products are obtained by precipitation in cold diethyl ether.

Peptides are further purified with a Jupiter Proteo 90 APhenomenex column (2050×25.0 mm) on an Armen Glider CPC preparatory phase HPLC to yield 90-95% purity, confirmed by analytical HPLC. For all RP-HPLC purifications, gradient solvent systems utilize Buffer A (water with 0.1% TFA) and Buffer B (acetonitrile with 0.1% TFA). A gradient of 15-45% buffer B over 30 min is used to purify all peptides and monomers. Pure products are then analyzed by electrospray ionization mass spectroscopy (ESI-MS) on a Bruker amaZon SL to confirm molecular weight.

Polymerization: PLPs are achieved by ring-opening metathesis polymerization (ROMP) under nitrogen gas in a glove box. Norbornene conjugated peptide monomers (e.g., 15.0 equiv., 30 mM) are dissolved in degassed DMF with 1M LiCl. Next, the olefin metathesis initiator (IMesH2)(C5H5N)2(Cl)2Ru=CHPh stock solution (e.g., 1.0 equiv., 20 mg/mL in DMF) is quickly added into the monomer solution. The solution is left to stir until the full consumption of monomers. In the case of rhodamine tagged polymers, this is achieved by the addition of 1 eq of a rhodamine linked to norbornene via a six-carbon chain linker with an amide bond. After the polymerization, the polymer solution is precipitated in diethyl ether, and is further purified via dialysis into deionized water. Finally, the polymer product is obtained by lyophilization. Aspects of this process are described in further detail in Kammeyer et al., Polymerization of Protecting-Group-Free Peptides via ROMP, Polym. Chem. 2013, 4 (14), 3929-3933 and Nomura et al., Precise Synthesis of Polymers Containing Functional End Groups by Living Ring-Opening Metathesis Polymerization (ROMP): Efficient Tools for Synthesis of Block Graft Copolymers, Polym. 2010, 51(9), 1861-1881, each of which is incorporated by reference herein in its entirety, and more specifically for methodologies of polymerization, to the extent not inconsistent with the description herein.

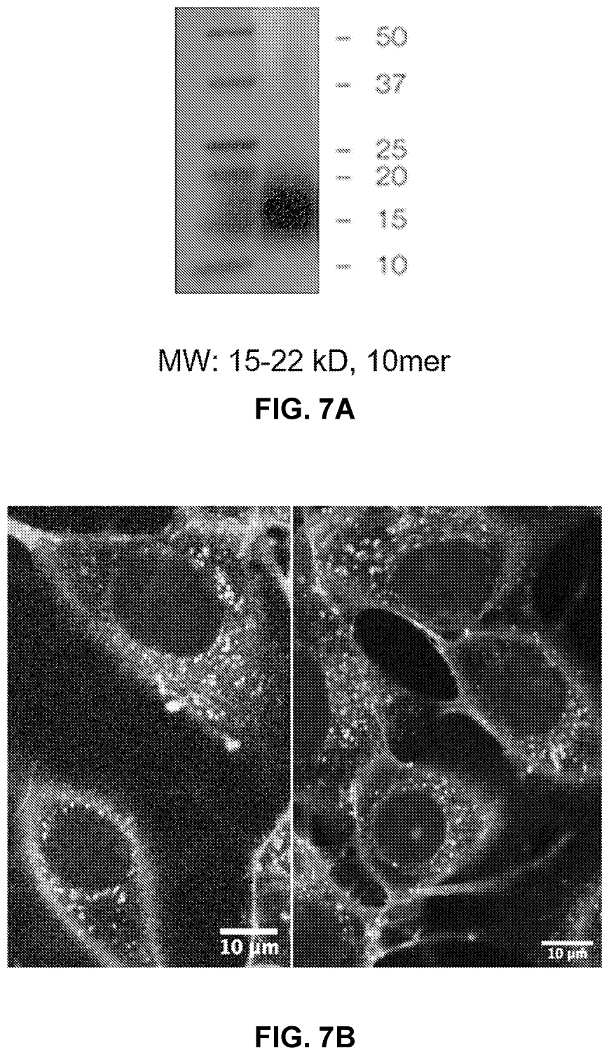

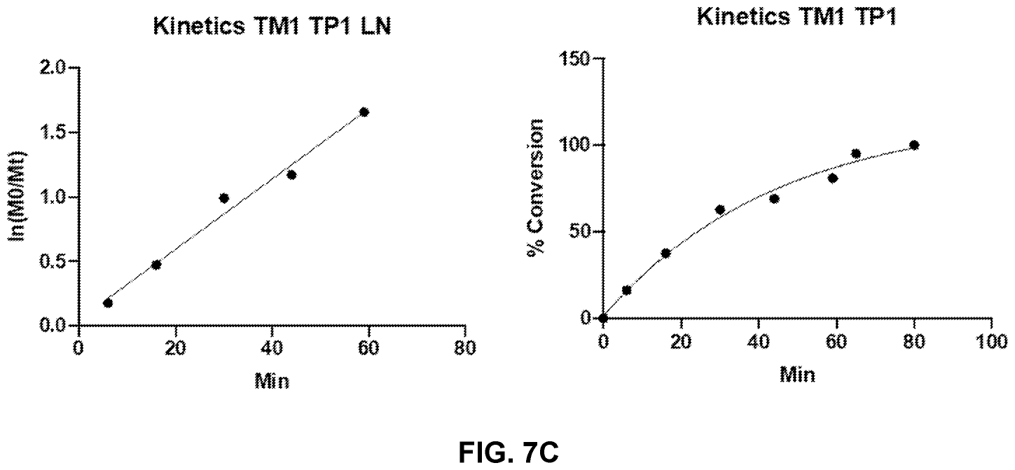

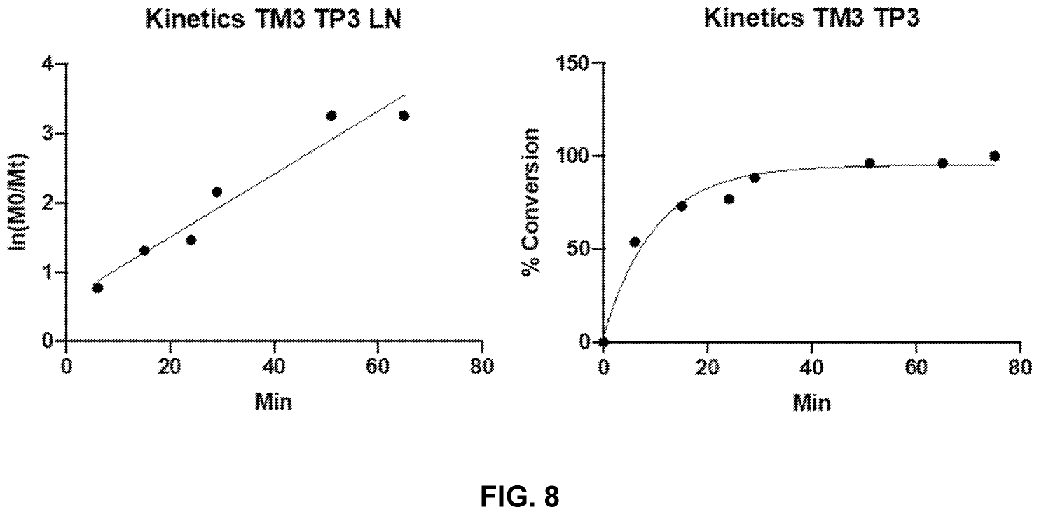

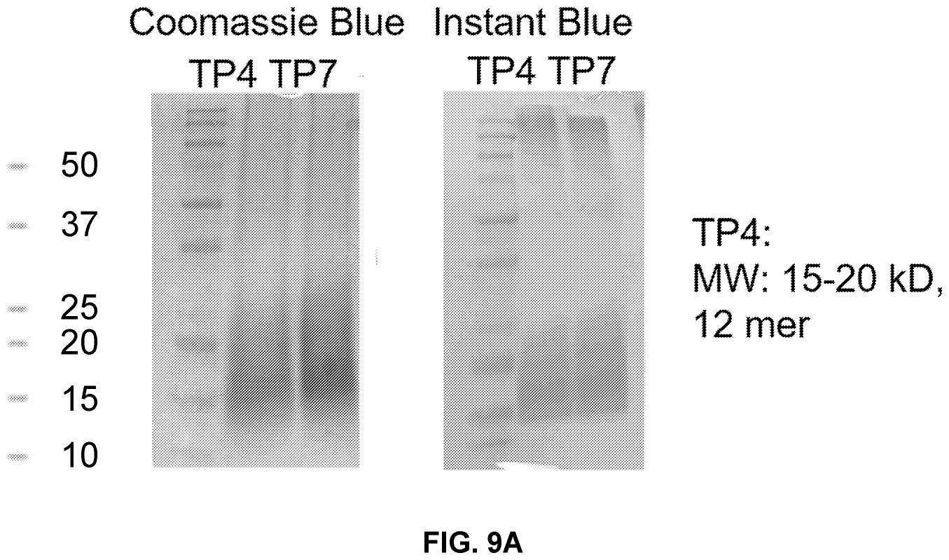

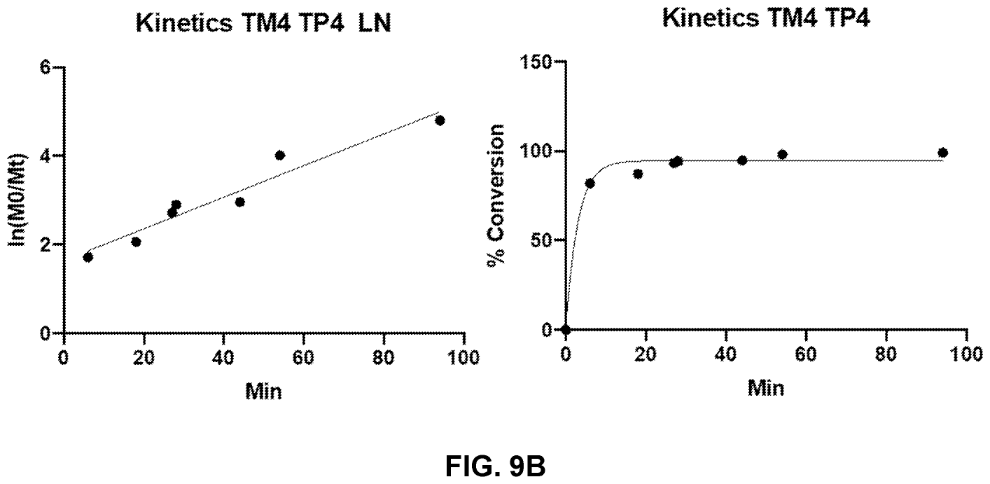

1H NMR is used to confirm complete consumption of the peptide monomer and to determine the time period required to reach completion. The percent conversion of monomer to polymer is tracked over time by comparing a decreasing monomer peak by NMR relative to an internal standard. The linear plot of the NMR reveals the pseudo-first-order kinetics (see e.g., FIG. 7C showing polymerization kinetics using SEQ ID: 16 as the peptide monomer; FIG. 8 using SEQ ID: 1; FIG. 9B using SEQ ID: 4 (“TP4”);

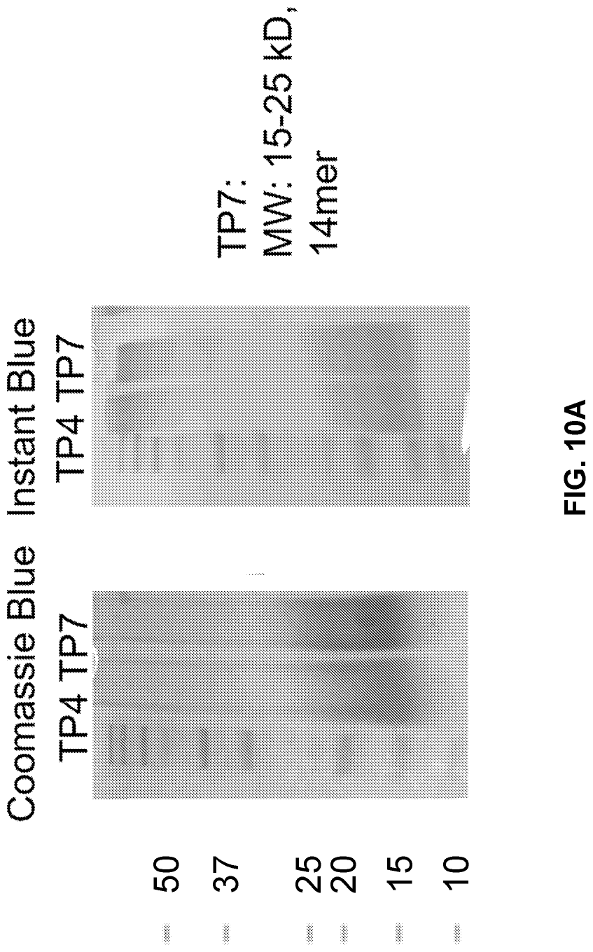

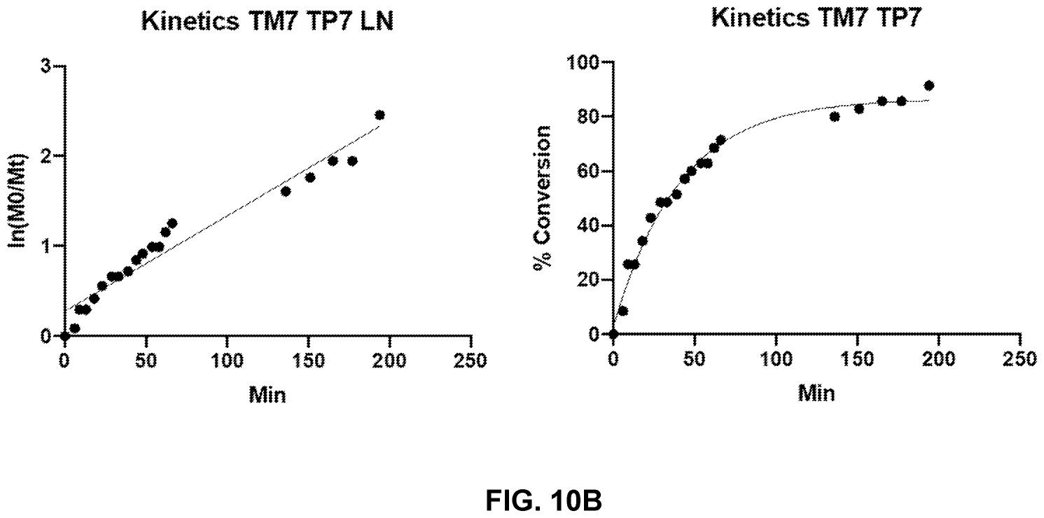

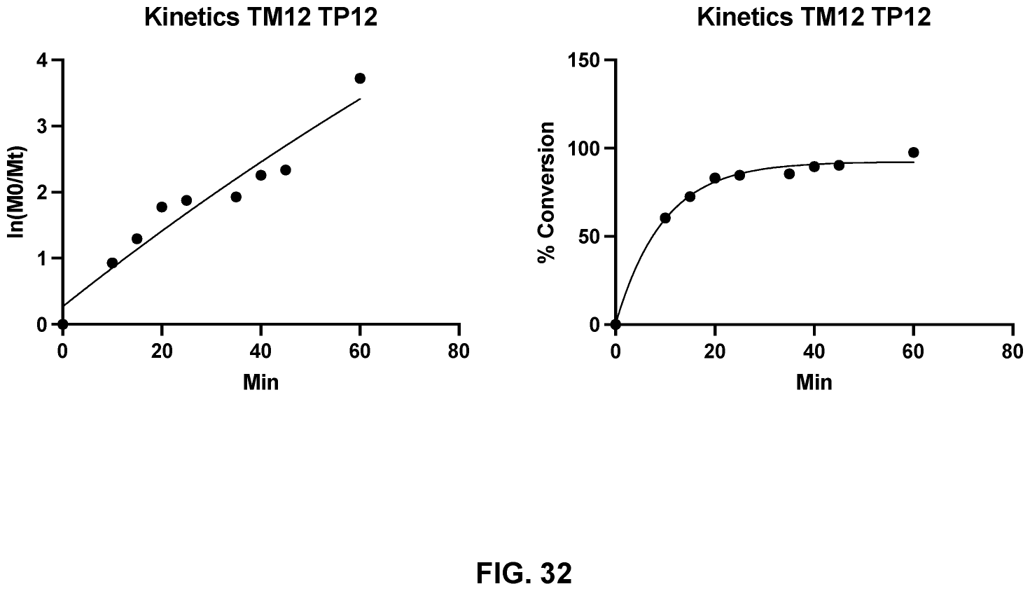

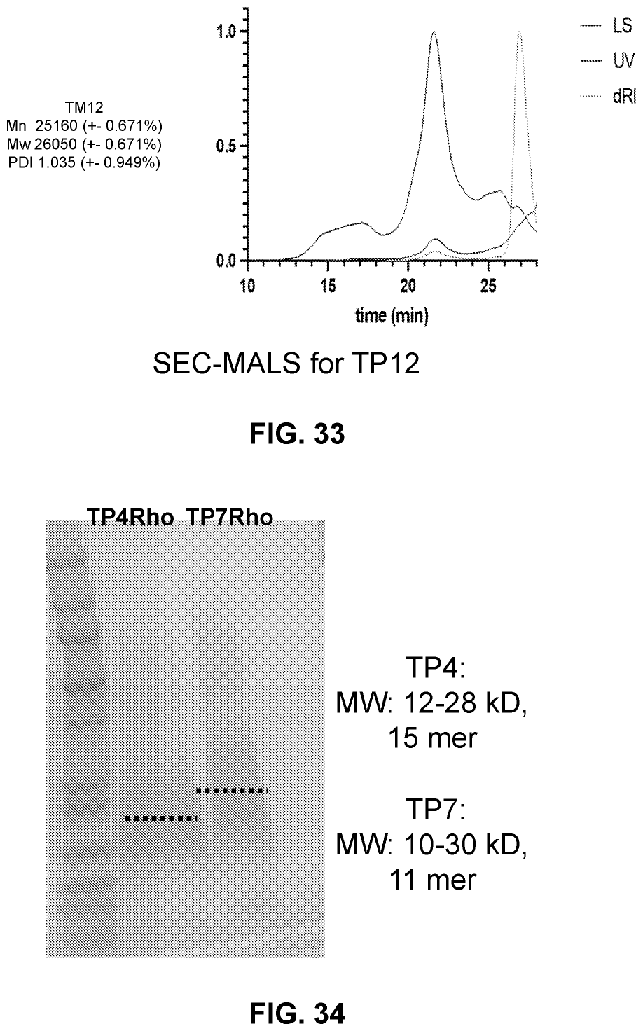

FIG. 10B using SEQ ID: 2 (“TP7”); FIG. 32 using SEQ ID: 10 (“TP12”)). The polymers are terminated with ethyl vinyl ether (10 eq) for 1 h at room temperature, precipitated and washed with cold diethyl ether three times and collected by centrifugation. Polymers molecular weight and polydispersity are determined by SEC MALS (Phenomenex Phenogel 5p 103A, 1K-75K, 300×7.8 mm in series with a Phenomenex Phenogel 5p 103A, 10K-100K, 300×7.8 mm) at 65° C. in 0.05M LiBr in DMF, using a ChromTech Series 1500 pump equipped with a multi-angle light scattering detector (DAWN-HELIOS II, Wyatt Technology) and a refractive index detector (Wyatt Optilab TrEX) normalized to a 30,000 MW polystyrene standard (see e.g., FIG. 33 showing SEC-MALS results for a PLP homopolymer incorporating SEQ ID: 10 (“TP12”)). Molecular weight may also be determined via SDS-PAGE (sodium dodecyl sulfate-polyacrylamide gel electrophoresis). Polymers are run at 2 mg/ml on precast Mini-PROTEAN TGX gels 4-10% and visualized using Instant Blue or Coomasie Blue Stain (see e.g., FIG. 7A showing molecular weight results for a PLP homopolymer (DP 10) using SEQ ID: 16; FIG. 9A using SEQ ID: 4 (“TP4”) and SEQ ID: 2 (“TP7”) at DP 12; FIG. 10A using SEQ ID: 4 (“TP4”) and SEQ ID: 2 (“TP7”) at DP 14).

### Example 1

This example describes the properties, design, and performance of an exemplary PLP for the investigation of protein phase separation.

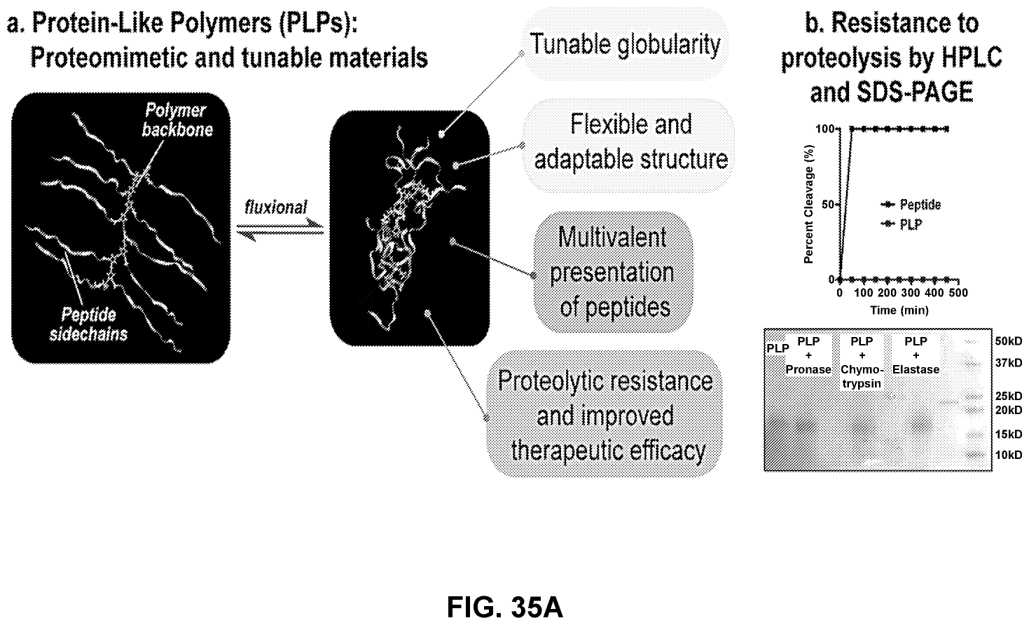

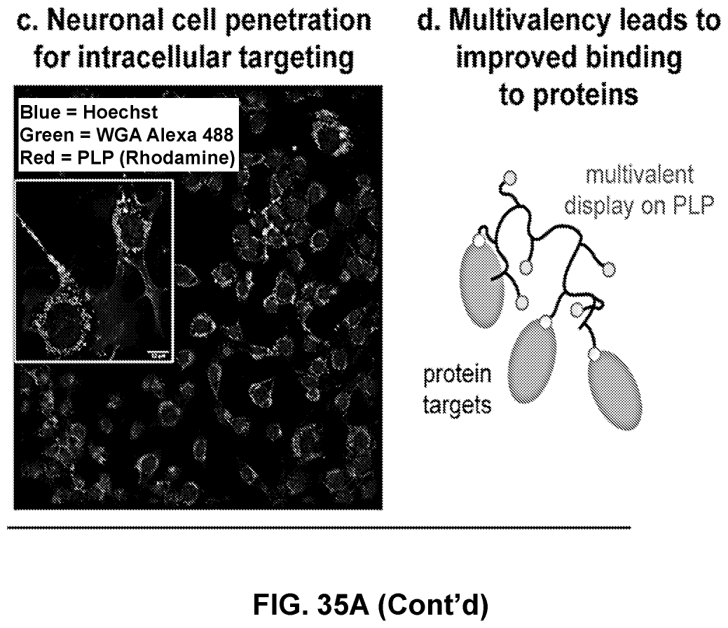

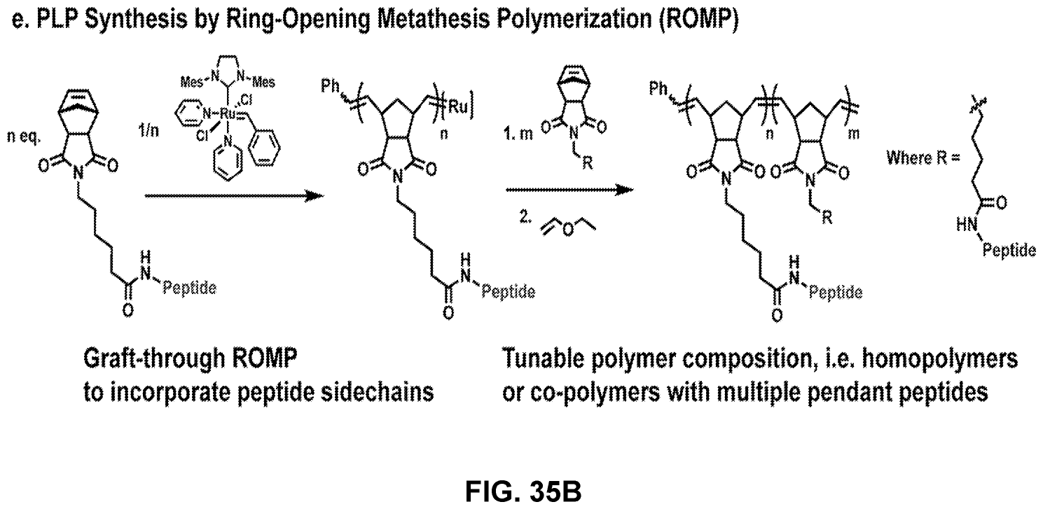

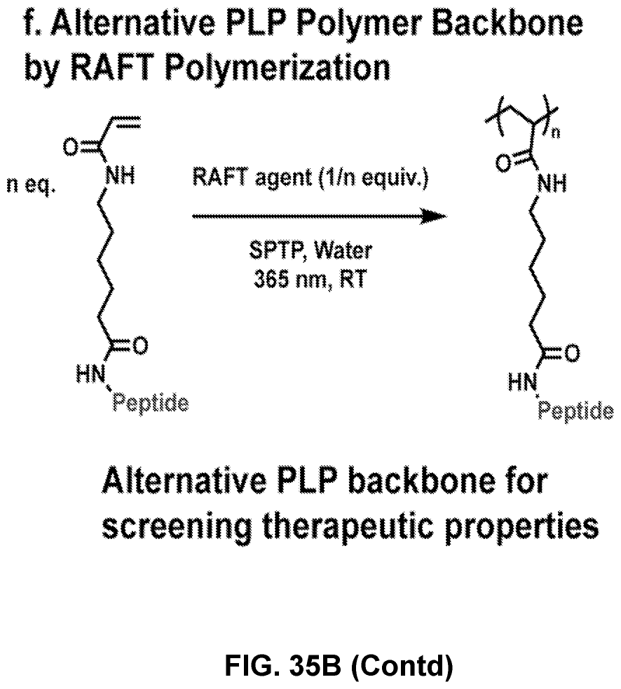

PLP Platform: In PLPs, a target peptide sequence is synthesized via SPPS followed by functionalization of the peptide with a reactive monomer moiety. The resulting monomer thus has a peptide side chain pendant to a polymerizable motif, which results in the formation of a brush polymer with a high density of peptide side chains formed via graft-through polymerization (FIG. 35B). The choice of the polymerizable unit and thus the polymer backbone has been shown to be flexible, although norbornene imide-based monomers are particularly effective due to their rapid polymerization kinetics by ring-opening metathesis polymerization (ROMP) (FIG. 35B). In solution, the hydrophobic backbone of PLPs collapses to form a globular single-chain, with a surface that is decorated with a high density of peptides. The globular structure and densely packed peptide corona minimizes the solvent accessible surface area of the peptides, inhibiting proteolysis, even for L-amino acids (FIG. 35A). Callmann C E et al., Poly(peptide): Synthesis, Structure, and Function of Peptide-Polymer Amphiphiles and Protein-like Polymers. Acc Chem Res 2020; 53:400-13; Gianneschi N C et al., Biomolecular Densely Grafted Brush Polymers: Oligonucleotides, Oligosaccharides and Oligopeptides. Angew Chemie Int Ed 2020; Blum A P, Kammeyer J K, Gianneschi N C. Furthermore, it is believed that the high density of peptides in the PLP creates a high degree of multivalency (FIG. 35A), as demonstrated by a 100-500 fold increase in binding equilibrium constants compared to the individual peptides. In addition, PLPs can be designed to penetrate cells, and have been shown to distribute in the cytosol where they exhibit bioactivity in multiple cell types from cancer cells, to neurons (FIG. 35A) and stem cells.

Peptide Side Chains: Here, the peptide side chain is selected such that it represents the intrinsically disordered region of proteins of high clinical interest. For example, the microtubule-binding region (MBTR) of tau (SEQ ID: 1 (VQIVYK), SEQ ID: 3 (VQIINK)). The peptide can be strategically altered in a bottom-up approach by adding or deleting certain amino acids, allowing the strength of peptide-peptide interactions and solubility to be controllably tuned. The resulting monomer can then be polymerized to form a graft polymer with a high density of peptide side chains, effectively using the synthetic toolkit of polymer chemistry to engineer a high degree of multivalency into a protein mimic. Furthermore, the peptide monomer can also be copolymerized with non-peptidic monomers to create statistical and block copolymers with tunable peptide grafting densities. The ability to tune the grafting density of peptide side chains, essentially the ability to tune the distance between each peptide side chain along the backbone, is expected to modulate the multivalency and, ultimately, the strength of protein-PLP interactions. We predict that the insight gained by performing such a systematic study within the bounds of well-established theories will provide paradigm-shifting perspectives on the key factors that drive LLPS for proteins.

### Example 2

TABLE A
List of peptide sequences for integration into embodiments of the PLPs described
herein. Charge is relative to the average pH in a human body
(i.e., between 7.35-7.45).

| Sequence | Protein | Charge | Expected Function |
|---|---|---|---|
| SEQ ID NO: 1 (VQIVYK) | Tau | 1 | R3 hexapeptide |
| | | | responsible for |
| | | | aggregation |
| SEQ ID NO: 2 | Tau | 3 | Modified R3 peptide |
| (DRVQIVYKRR) | | | responsible for |
| | | | aggregation |
| SEQ ID NO: 3 (VQIINK) | Tau | 1 | R2 hexapeptide |
| | | | responsible for |
| | | | aggregation |
| SEQ ID NO: 4 (VQIINKRR) | Tau | 3 | Modified R2 peptide |
| | | | responsible for |
| | | | aggregation |
| SEQ ID NO: 5 | Tau | 4 | R2 hexapeptide |
| (KVQIINKKLDRR) | | | responsible for |
| | | | aggregation |
| SEQ ID NO: 6 | Microtubulin | −3 | Tau-binding |
| (YQQYQDATADEQG) | | | microtubulin mimic |
| SEQ ID NO: 7 | Microtubulin | 0 | Modified Tau-binding |
| (YQQYQDATADEQGRRR) | | | microtubulin mimic |
| SEQ ID NO: 8 (WMINK) | Tau Inhibitor | 1 | Inhibitor of |
| | | | aggregation |
| SEQ ID NO: 9 (WMINKRR) | Tau Inhibitor | 3 | Modified inhibitor of |
| | | | aggregation |
| SEQ ID NO: 10 (VQPINK) | Tau Inhibitor | 1 | Inhibitor of |
| | | | aggregation |
| SEQ ID NO: 11 (ALAPYIP) | E3 Ligase | 0 | E3 ligase recruiter |
| SEQ ID NO: 12 | E3 Ligase | 2 | Modified E3 ligase |
| (ALAPYIPRR) | | | recruiter |
| SEQ ID NO: 13 | Tau | 3 | Modified R3 peptide |
| (VQIVYKRR) | | | responsible for |
| | | | aggregation |
| SEQ ID NO: 14 | Tau Inhibitor | 3 | Modified inhibitor of |
| (DRWMINKRR) | | | aggregation |
| SEQ ID NO: 15 (VQPINKR) | Tau Inhibitor | 2 | Modified inhibitor of |
| | | | aggregation |
| SEQ ID NO: 16 | Microtubulin | −1 | Modified Tau-binding |
| (YQQYQDATADEQGRR) | | | microtubulin mimic |

### Example 3

This example provides exemplary cell permeability assays for certain PLPs.

TABLE B
Characterizations of PLPs used in cell permeability assays.

| | MW of | | | | | MW of | |
|---|---|---|---|---|---|---|---|
| Peptide | monomer | Monomer | mM in | Target | Actual | polymer | Polymerization |
| monomer | (g/mol) | mg | LiCl | DP | DP | (kD) | time (min) |
| SEQ ID: 4 | 1287.23 | 26.54 | 30 | 15 | 15 | 20 | 90 |
| SEQ ID: 2 | 1593.55 | 21.9 | 30 | 15 | 11 | 20 | 195 |
| SEQ ID: 4 | 1287.23 | 99.51 | 30 | 15 | 15 | 20 | 90 |
| SEQ ID: 2 | 1593.55 | 123.2 | 30 | 15 | 13 | 21 | 195 |
| SEQ ID: 16 | 2089.25 | 46.2 | 30 | 15 | 10 | 21 | 65 |

Table B results derived in part from FIG. 34 showing the SDS-PAGE results for rhodamine labelled PLPs incorporating either SEQ ID: 4 (i.e., “TP4Rho” of FIG. 34) or SEQ ID: 2 (i.e., “TP7Rho” of FIG. 34).

Mouse striatal cells HdhQ7/7 from Coriell Institute were plated in 4-chamber 35 mm round glass-bottom dishes at a density of 50,000 per well. Cells were incubated for 24 hours in a 5% CO₂ atmosphere at 33° C. Rhodamine labeled PLPs, rhodamine dye, and vehicle control in 10% FBS DMEM medium without phenol red were incubated with the cells for 24 hours, respectively. The cell nuclei (stained with Hoechst) were accomplished using a 405 nm laser with a 15% laser power. The cell membrane (stained with Wheat Germ Agglutinin, Alexa Fluor 488 Conjugate) was accomplished using a 488 nm laser with a 12% laser power. Cell imaging for Rhodamine fluorescence was accomplished using a 581 nm laser with an 8% laser power.

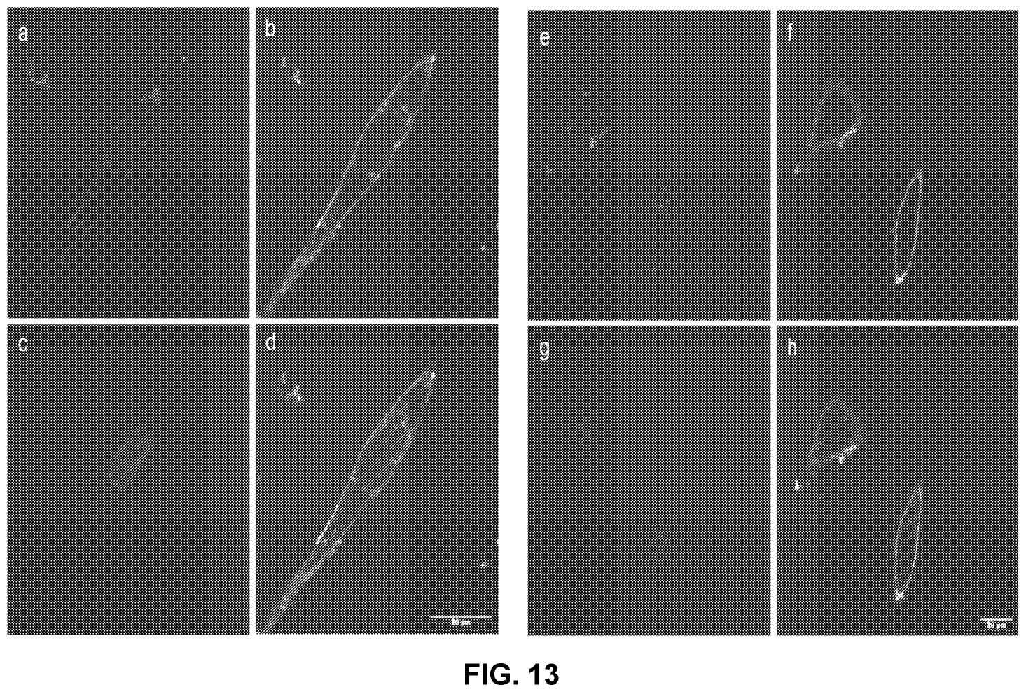

Each of the polymers listed in Table B demonstrated the capability to permeate the striatal cells, as shown in FIG. 7B (SEQ ID: 16) and FIG. 13 (left: SEQ ID: 4; right: SEQ ID: 2). It is hypothesized that permeation is facilitated, in part, by modifying the known structures of tau and microtubulin with additional residues, notably arginine, to aid in solubility.

### Example 4

It is hypothesized that PLPs capable of liquid-liquid phase separation (LLPS) may be better suited to engage with phase separated tau. Accordingly, this example demonstrates LLPS of certain rhodamine labeled PLPs.

Solutions were dissolved in HEPES buffer: 10 mM HEPES buffer (pH 7.4) with 0.1 mM EDTA and 2 mM DTT, Media buffer: Dulbecco Modified Eagles Medium (high glucose) with 2 mM L-glutamine with 10% Fetal Bovine Serum, 1% Penicillin/Streptomycin, or water. Different crowding agents were also tested using HEPES buffer—PEG and heparin. For heparin, stock solutions of 10 μM of each rhodamine labeled PLP (using either SEQ ID: 4 or SEQ ID: 7 as peptide monomers) and 30 μM of heparin sodium salt were prepared in buffer and kept refrigerated. Before experiments, solutions were given at least 30 min to come to room temperature and experiments were carried out at room temperature. In the microscope room, 20 μL of PLP was added to 5 μL of heparin and mixed gently in a microcentrifuge tube. 5 μL of solution was immediately pipetted onto Fisherbrand glass microscope slides and coverslip applied. All reactions were visualized on Olympus BX53 Fluorescent Microscope using the 581 nm laser to monitor the rhodamine, as well as brightfield. LLPS occurred within 60 min of initiation.

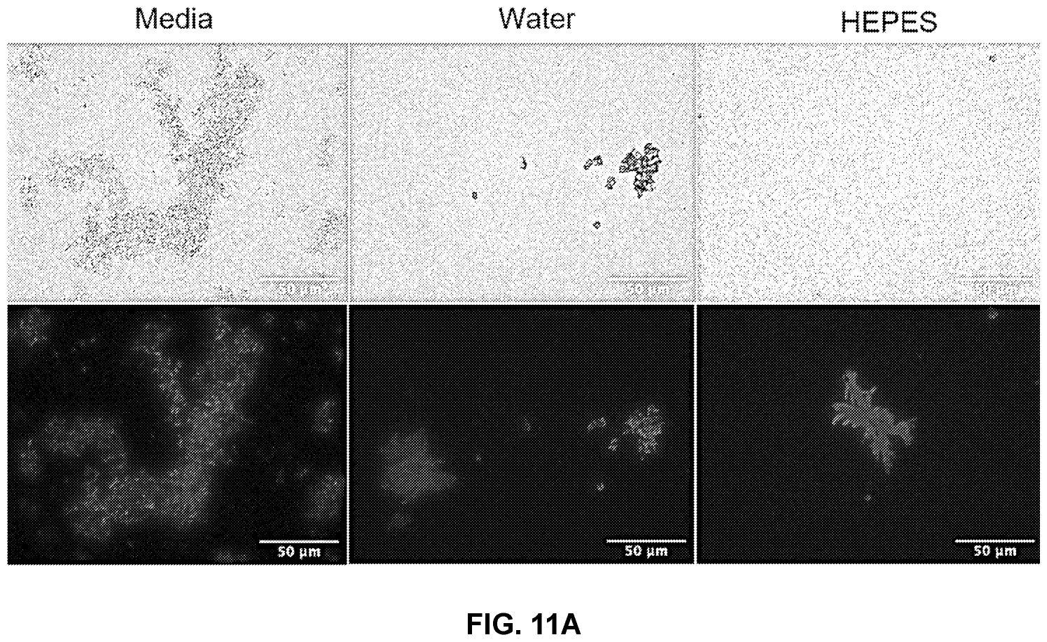

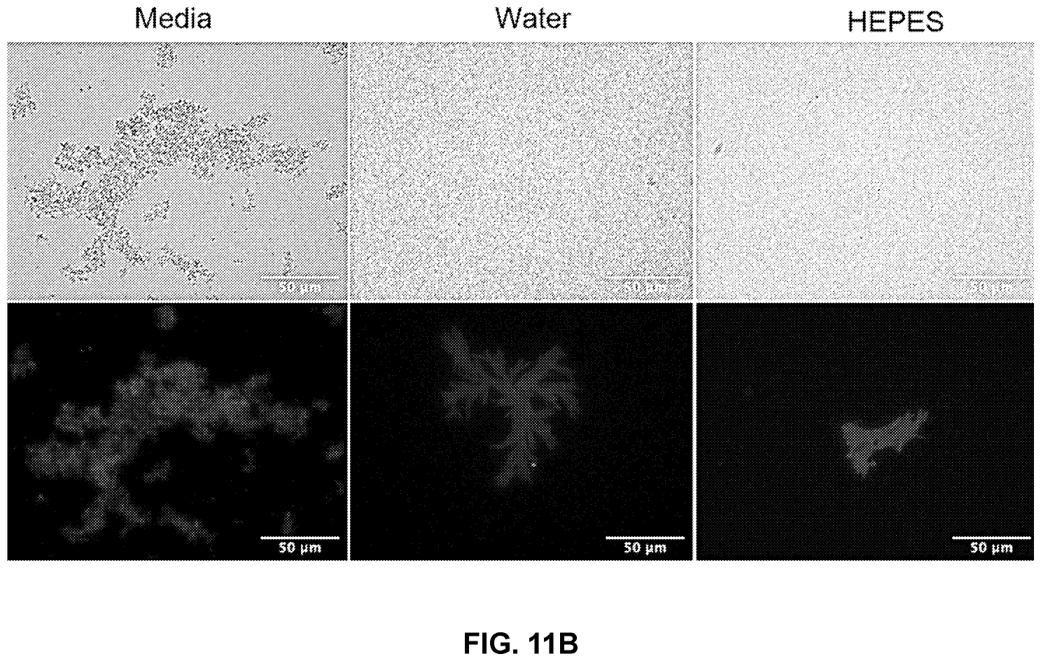

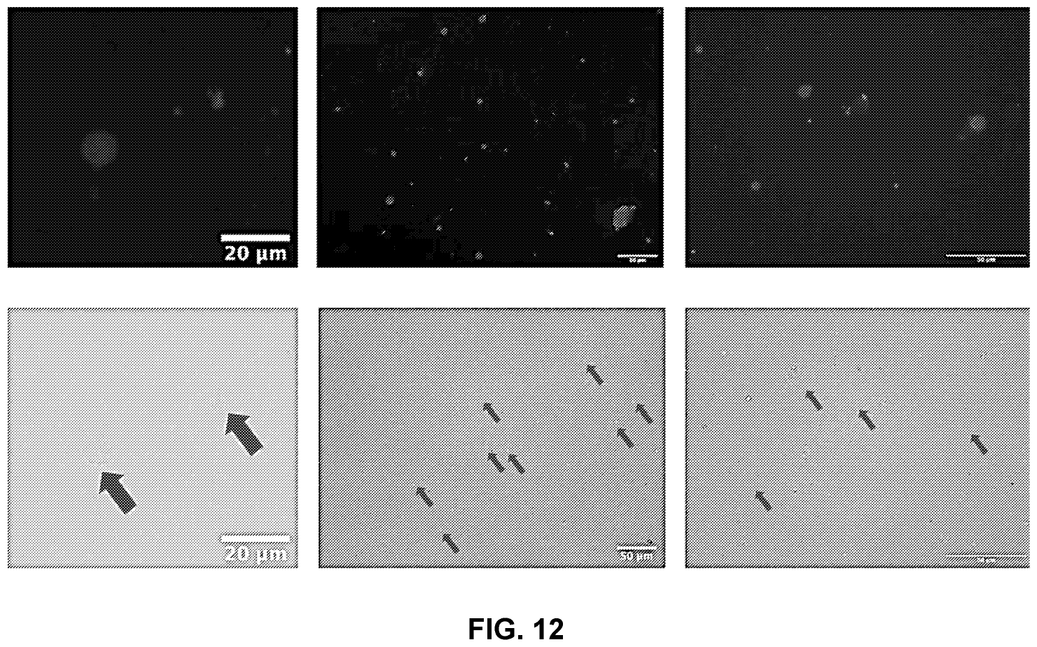

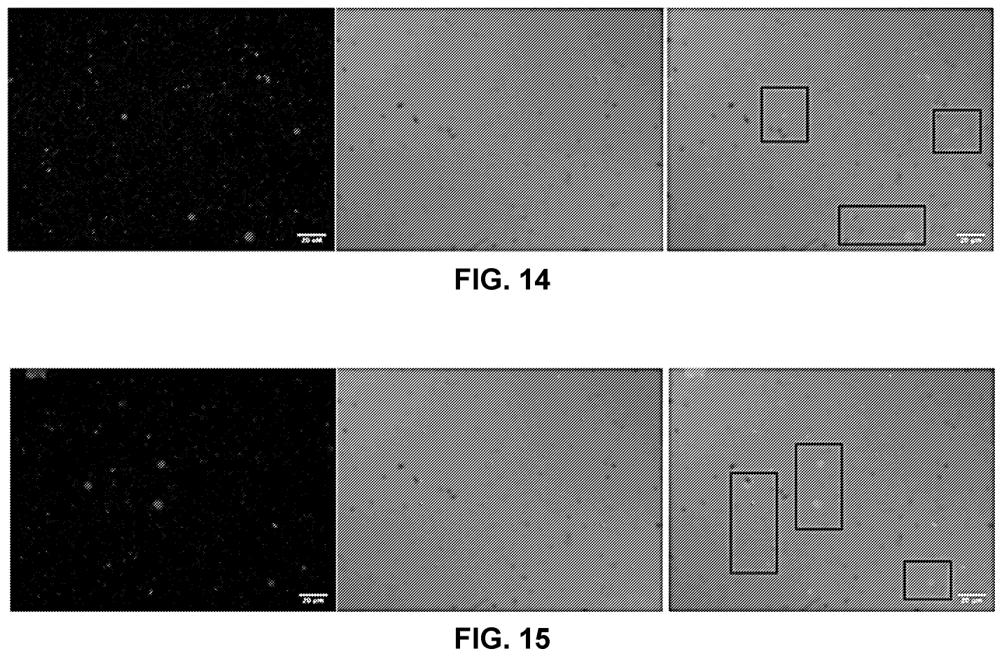

FIG. 11 A-11B demonstrates the impact of different solvents on the LLPS results for TP4 (FIG. 11 A) and TP7 (FIG. 111B)—with increased salt concentration appearing to induce a salting out effect seen in proteins, and HEPES buffer appearing to facilitate aggregates. For samples tested with PEG, the crowding agent appeared to induce LLPS phase condensates of a PLP homopolymer incorporating SEQ ID: 4 as peptide sidechains, as shown in FIG. 12. LLPS results for solutions in HEPES buffer with heparin are shown in FIG. 14 (SEQ ID: 4) and FIG. 15 (SEQ ID: 2) suggest that both PLP homopolymers tested are capable of liquid-liquid phase separation.

### Example 5

Based on the results acquired in Example 4, this Example 5 evaluated whether the same PLPs reacted in the same, or substantially similar, way with thioflavin T as does tau.

All solutions were dissolved in HEPES buffer: 10 mM HEPES buffer (pH 7.4) with 0.1 mM EDTA and 2 mM DTT. Stock solutions of 10 μM of each rhodamine labeled PLP (using either SEQ ID:4 or SEQ ID: 7 as peptide monomers), 30 μM of heparin sodium salt, and 100 μM of Thioflavin T were prepared in HEPES buffer and kept refrigerated. Before experiments, solutions were given at least 30 min to come to room temperature and experiments were carried out at room temperature. 20 μL of PLP was added to 5 μL of heparin and 1 μL of thioflavin T and mixed gently in a microcentrifuge tube. The tube was kept stationary at room temperature for 24 hours, and then 5 μL of solution was pipetted onto Fisherbrand glass microscope slides and coverslip applied. Results were visualized on using the 581 nm laser to monitor the rhodamine, 488 nm laser to visualize the thioflavin T, as well as brightfield. Images were taken on Leica DM6B Fluorescent Microscope as Olympus microscope was removed from facility.

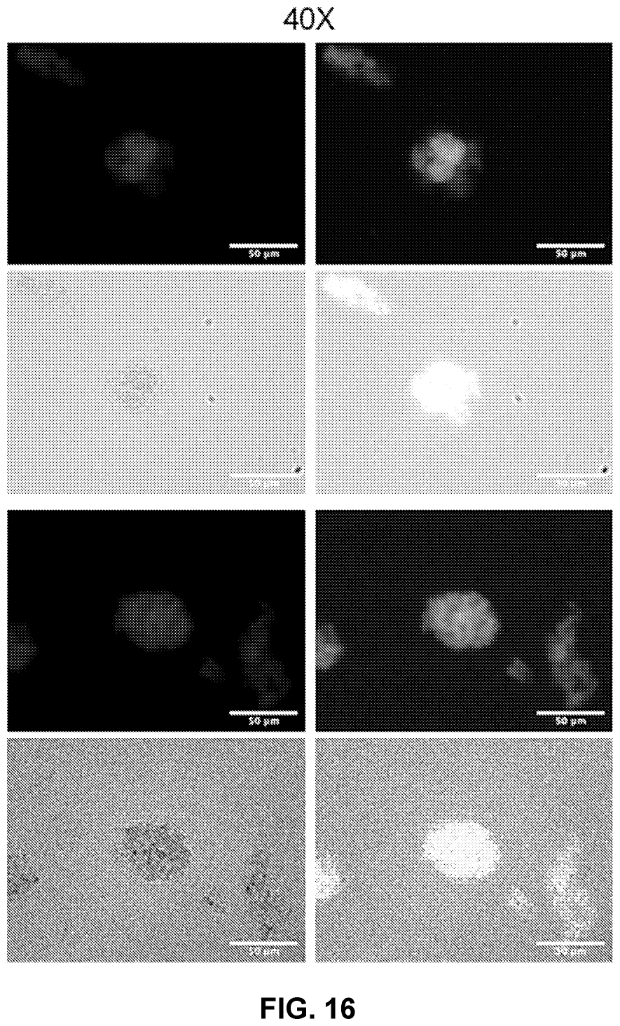

As depicted in FIG. 16, these results suggest that the tested PLPs are capable of reacting with thioflavin T in the same manner as tau, which is through binding to the beta sheet forming residues.

### Example 6

Following from the results of Examples 4 and 5, PLP homopolymers were subjected to additional testing to evaluate their material properties.

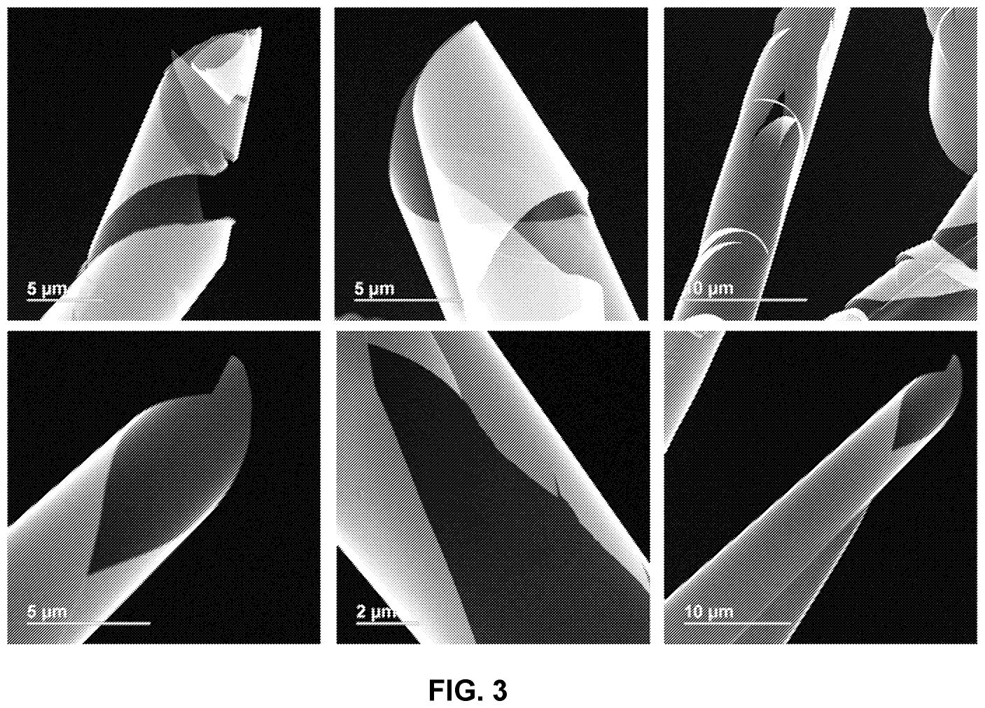

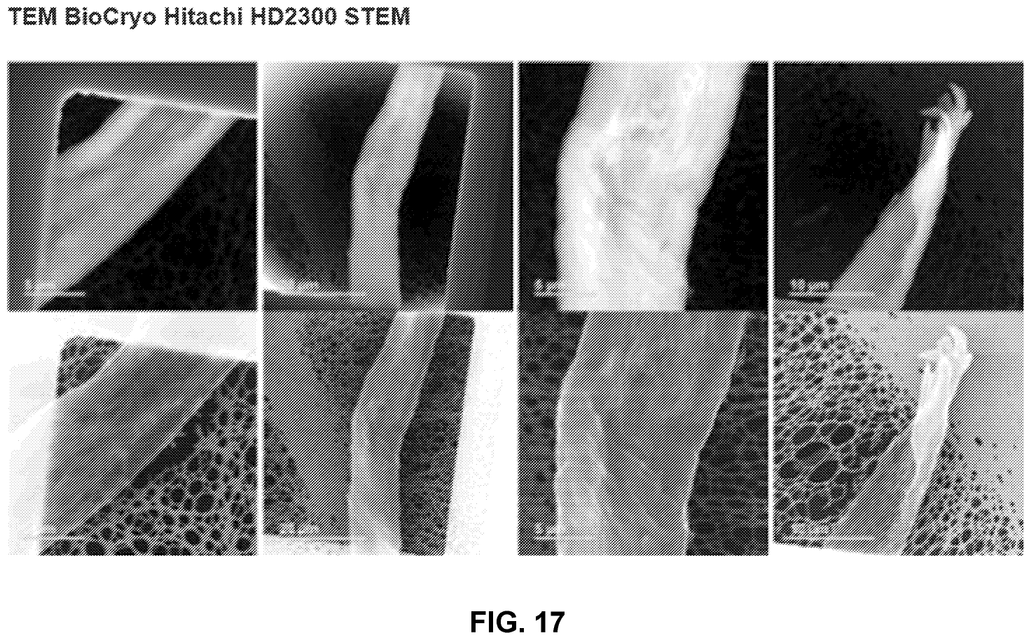

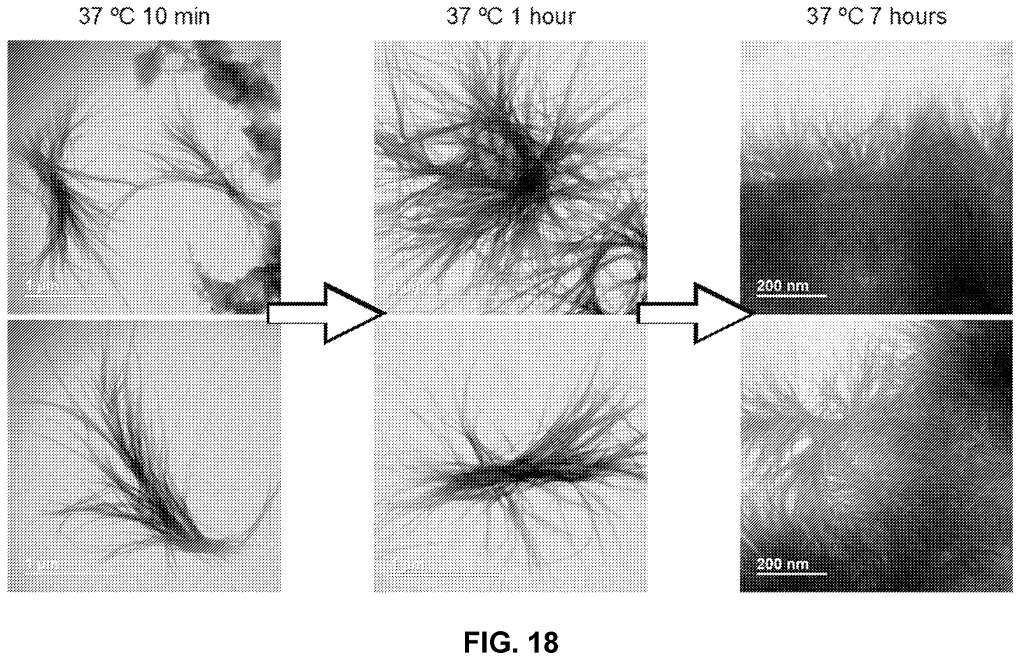

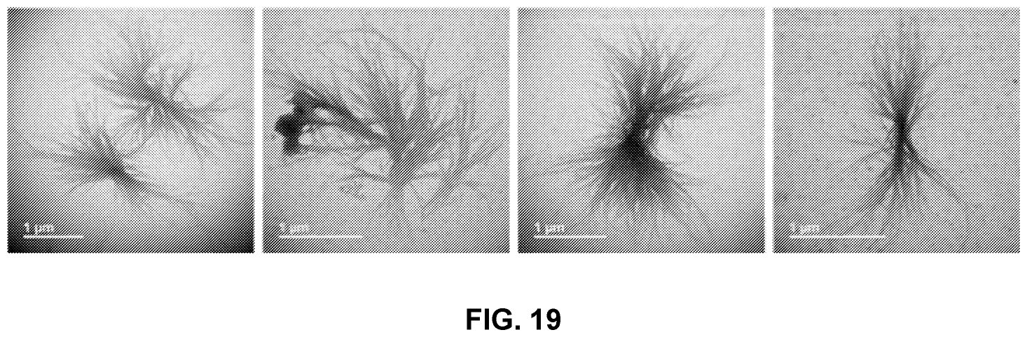

Using standard STEM protocols, PLPs having SEQ ID: 2 or SEQ ID: 4 as peptide monomers were prepared in H₂O and visualized. Initial STEM results in FIG. 3 appear to demonstrate beta sheet conformations (SEQ ID: 4 (top), SEQ ID: 2 (bottom)). Further, from the results depicted in FIG. 17, the homopolymer the beta sheet forming nature was confirmed. After putting the PLPs in HEPES LLPS buffer and heating to 37° C. (i.e., body temperature), a time course established the fibrilization of the polymer on dry state TEM. Images were captured at 10 minutes, 1 hour, and 7 hours as shown in FIG. 18. The same fibrilization was seen on purified K18 Tau protein 24 hours after incubation with heparin in PBS as depicted in FIG. 19. The remarkably similar characteristics was a testament to the mimetic capabilities of the PLP.

The proteomimetic similarities were further investigated using circular dichroism. Samples tested included K18 Tau, PLPs having SEQ ID: 4, SEQ ID: 2, and SEQ ID: 10, as well as said PLPs with K18 Tau. Each of these samples were dissolved in Tris buffer (10 mM Tris, 50 mM NaF, 0.5 mM dithiothreitol) and pH was adjusted to 7.4. Samples were tested with and without the addition of heparin. For samples containing heparin, samples were incubated in the presence of 10 μM heparin sodium salt for 24 hours. Polymers and peptides were run at 37° C., at a concentration of 3 mg/10 ml and K18 Tau was run at 10 uM. Parameters were: 1 mm quartz cuvette, continuous scan at 10 nm/min over 260 to 200 nm, 3 acquisitions averaged for one trace.

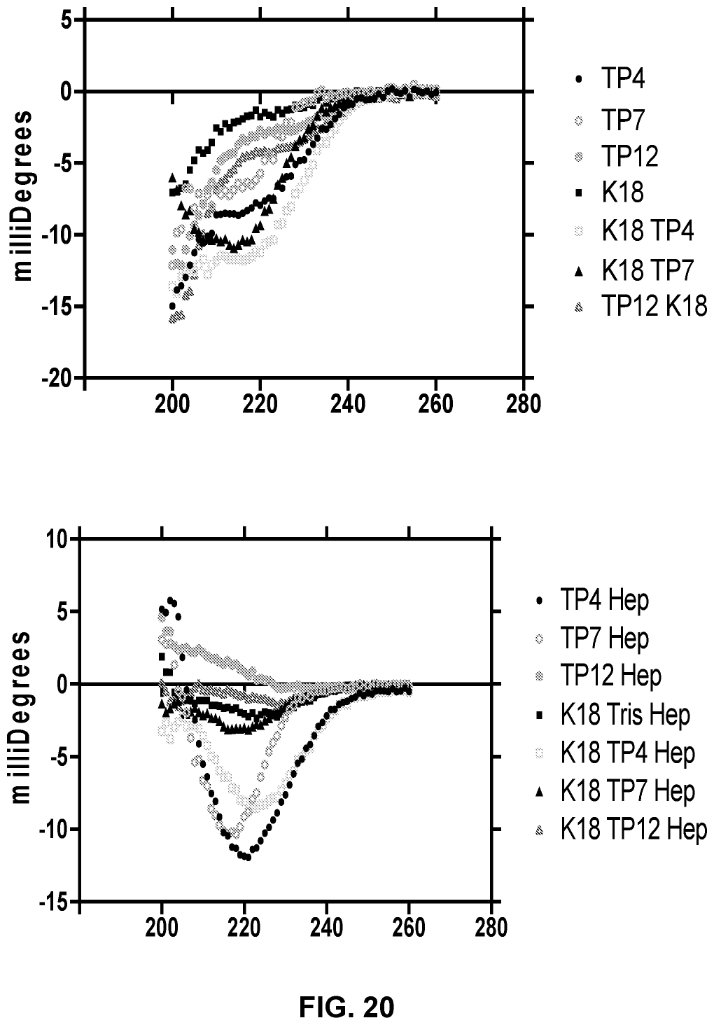

Samples without the addition of heparin displayed CD results consistent with random coil natures (FIG. 20, top). When combined with K18, the PLPs appeared to maintain random coil characteristics (FIG. 20, top). However, for samples including heparin, the CD results depicted beta sheet conformations (FIG. 20, bottom). As evidenced by circular dichroism with a peak near 220 nm for PLP-SEQ ID:4 in FIG. 20, it is believed the heparin induced aggregation and the formation of beta sheets. It is of interest to note that there seems to be an interaction between K18 and polymers where the extent of beta sheet character decreases. This was puzzling and was put aside to explore further in later experimentation (discussed in Example 7 below).

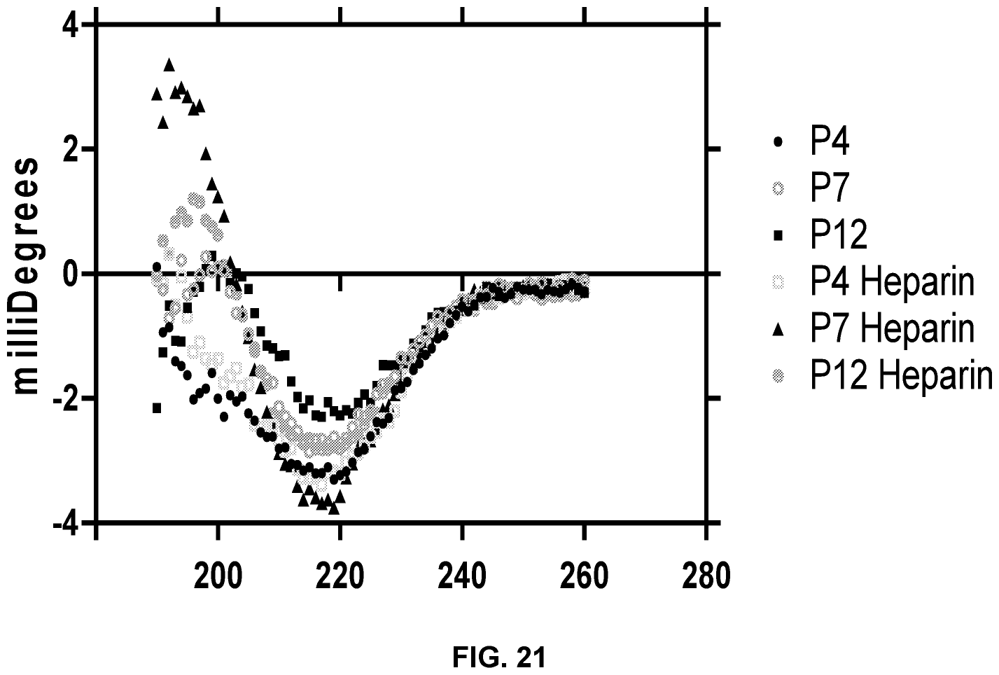

The free peptides (i.e., peptides characterized by SEQ ID: 4, SEQ ID: 2, or SEQ ID: 10) were synthesized as controls and run on circular dichroism. As portrayed in FIG. 21, each of the free peptides resulted in CD characterizations consistent with beta sheet formation—irrespective of the addition of heparin. These results suggested the polymer is inhibiting beta sheet formation unless heparin is introduced as a crowding agent. These results were inconsistent with our hypothesis and would be further examined in the Thioflavin T kinetics assays (discussed in Example 7 below).

### Example 7

Based on the unexpected results derived from Example 6, Thioflavin T kinetics assays were conducted to establish whether the free peptides had the same or substantially similar effects when incorporated into the PLP platform.

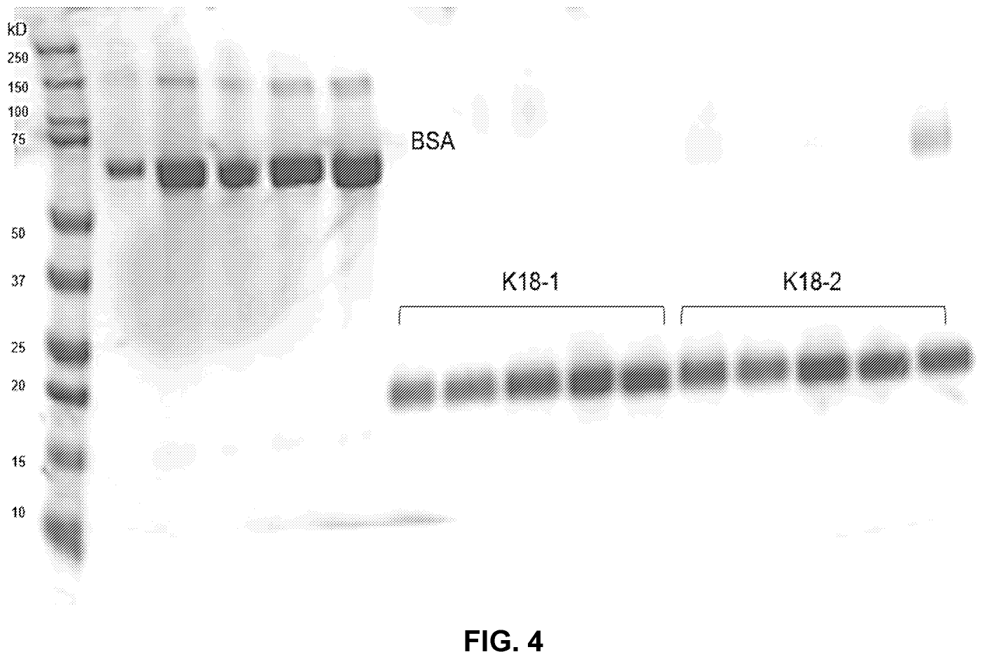

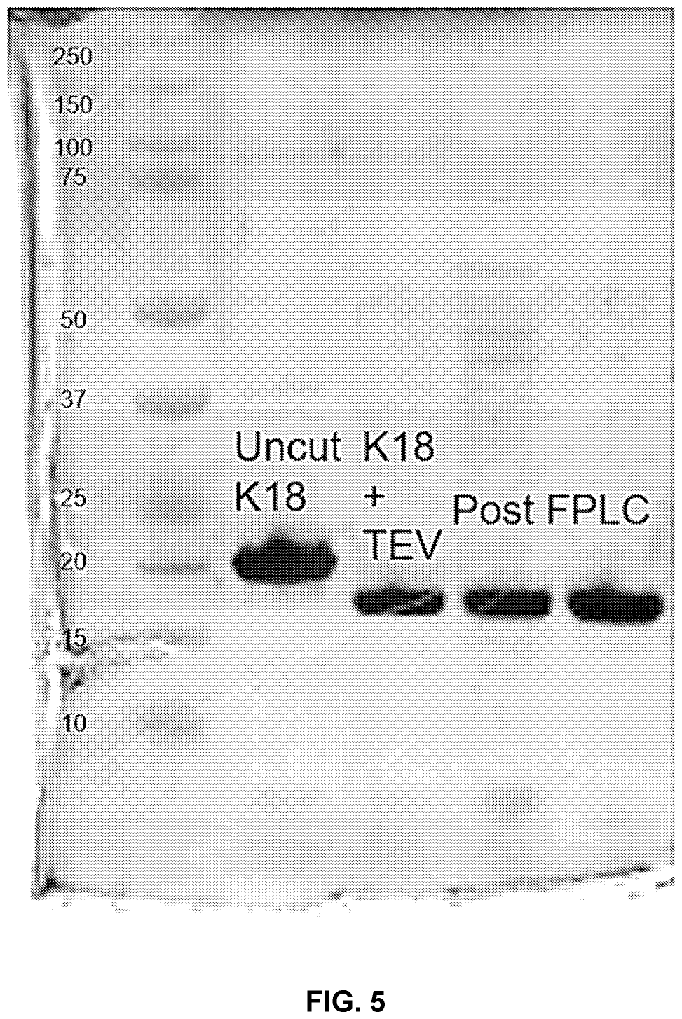

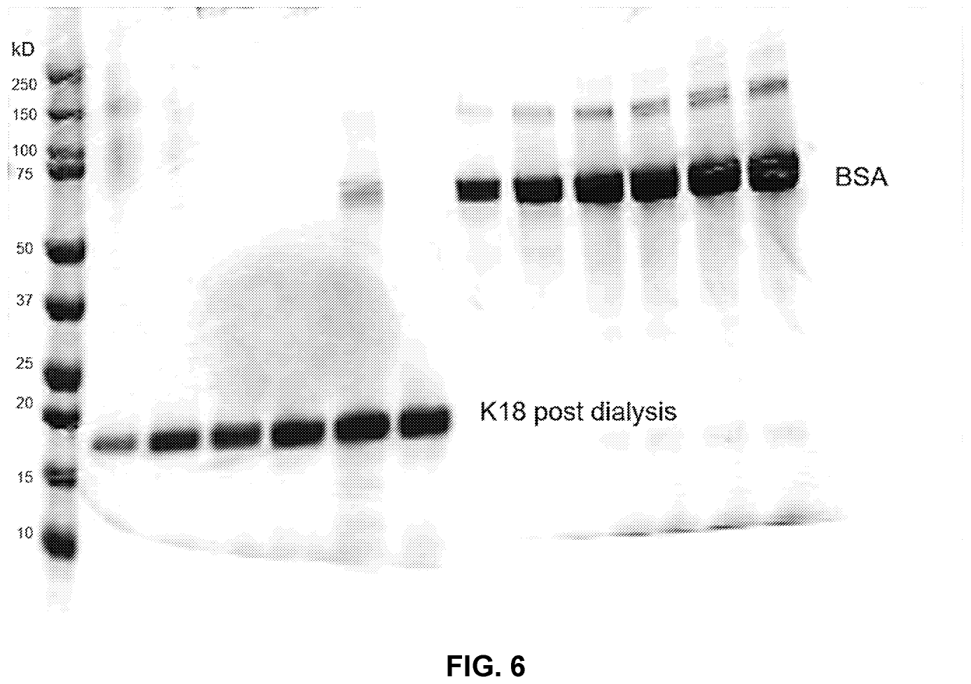

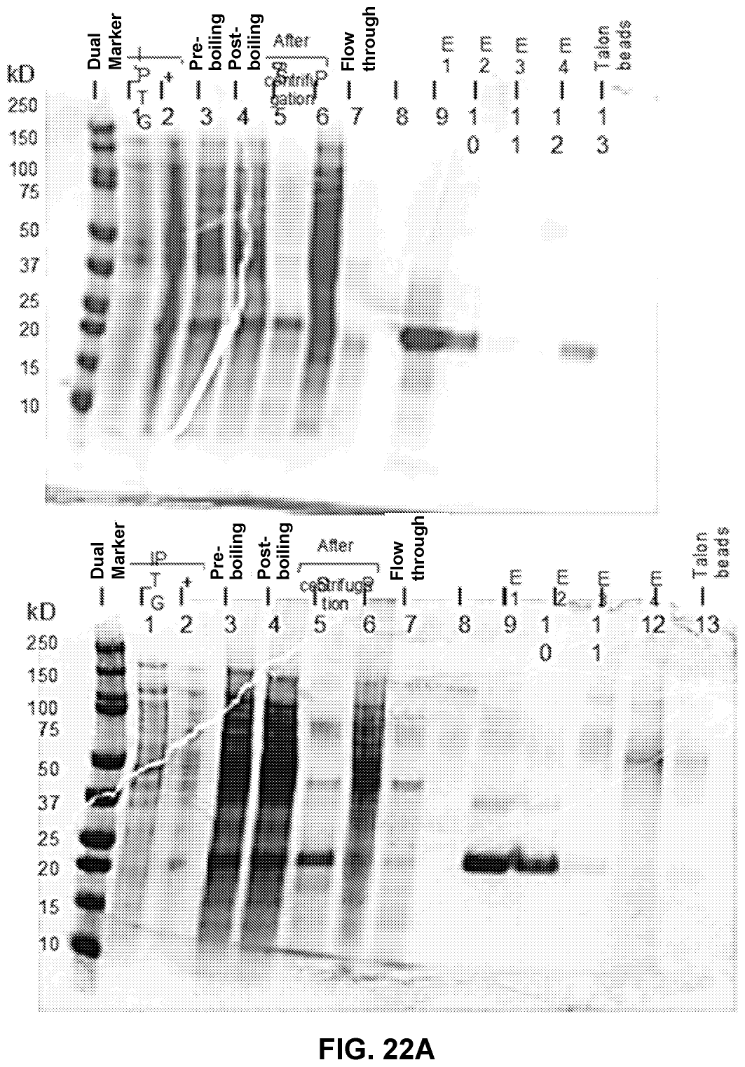

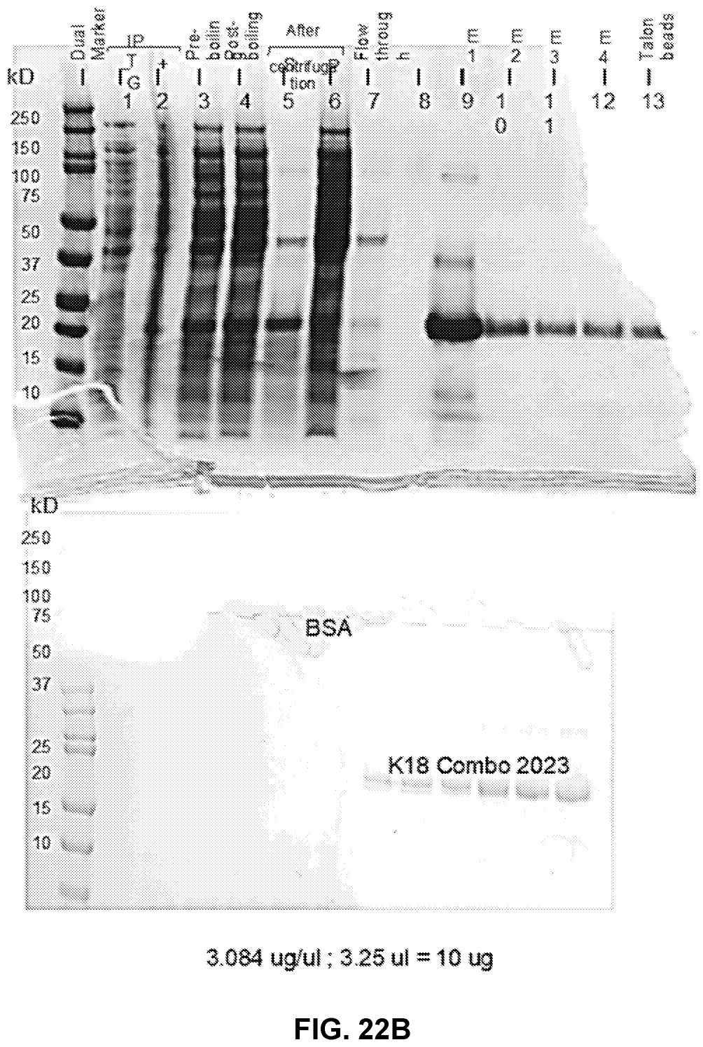

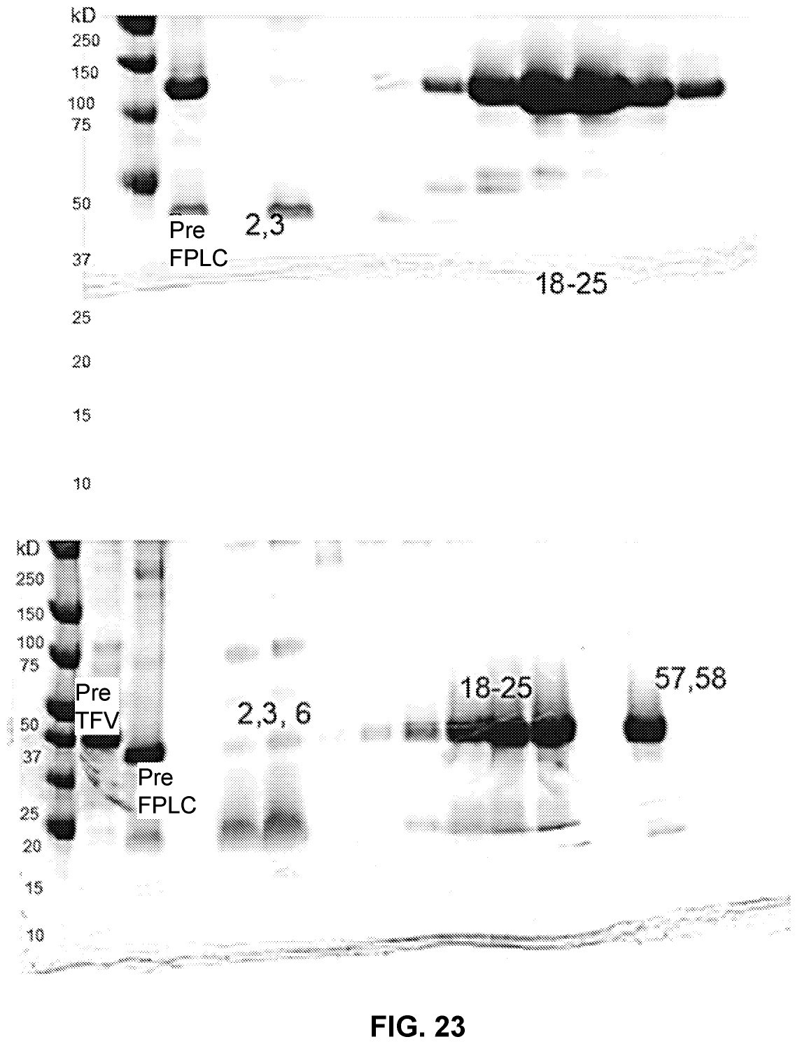

K18 Tau Expression and Purification: Before K18 Tau kinetics could be conducted, K18 Tau was first expressed, collected, and purified. The expression and collection of K18 Tau, combined with multiple days of purification, yielded a large quantity of K18 (see FIG. 22A-22B for purification and quantification results (a previous iteration is shown in FIG. 4)). However, this K18 was tagged with a His tag in order to collect/purify the K18 on a nickel bead column. Accordingly, TEV protease was used to cleave the His tag off the K18. Once cleaved, K18 was run over an FPLC column in two iterations due to size limitations of the column as shown in FIG. 23. The two iterations were then combined and subjected to dialysis according to standard dialysis procedures. The pooled fraction was quantified using BSA quantification (FIG. 6) resulting in 28 mg of purified K18 Tau. FIG. 5 demonstrates a silver stain for purity of K18, where the uncut K18 refers to His-tagged K18.

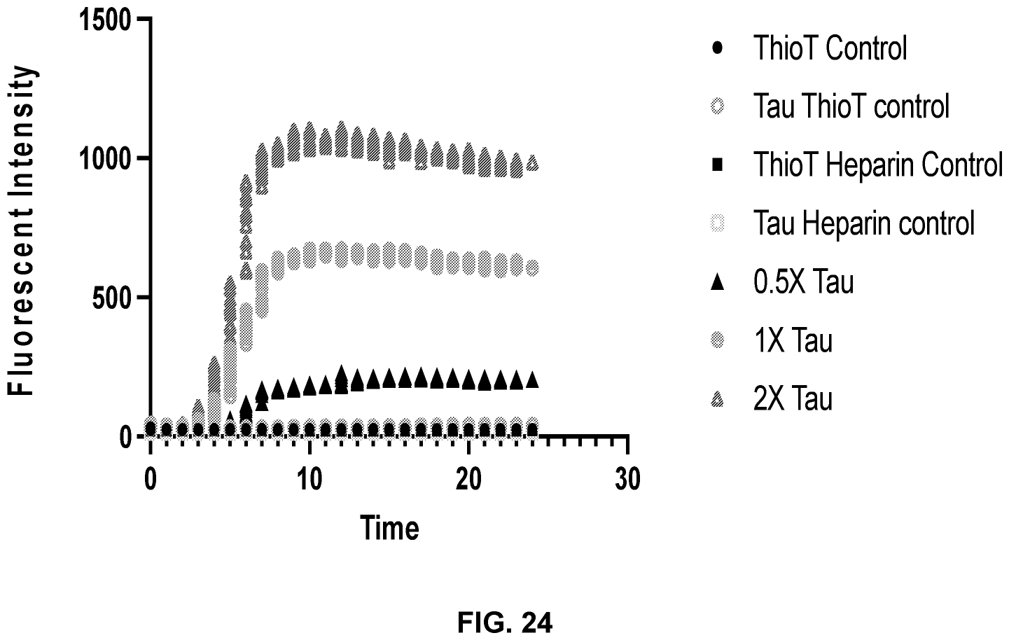

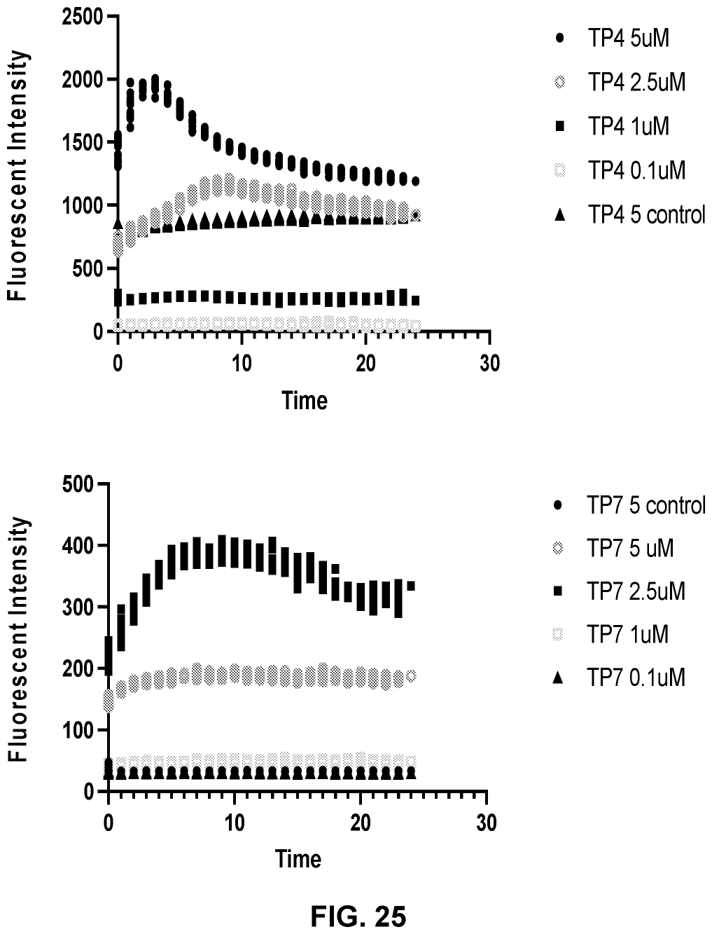

Tau Kinetics Thioflavin T Plate Reader Assay Protocol: First, controls and the effects of varying concentrations of K18 Tau were examined (FIG. 24). In FIG. 24, 1X Tau refers to 10 μM K18 Tau final concentration in the well with 100 μl total volume (i.e., 0.5× Tau refers to 5 μM K18 Tau and 2× Tau refers to 20 μM K18 Tau). Before PLPs were added to K18 Tau, the concentration at which the mimetic aggregative polymers would not activate Thioflavin T was evaluated. Based on the results depicted in FIG. 25, a PLP concentration of 1 μM was selected for further experimentations. It is noted that for TP4, 1 μM still produced a significant signal (FIG. 25, top), especially when compared to 1 μM of TP7 (FIG. 25, bottom) which portrayed negligible signal. Given the goal to investigate effects of the PLPs, it was determined that too high of a concentration could easily be adjusted, whereas too low of a concentration could result in a false negative with respect to impact.

PLPs incorporating SEQ ID: 4 (“TP4”), SEQ ID: 2 (“TP7”), or SEQ ID: 10 (“TP12”) as peptide sidechains were tested. The PLPs, K18 Tau (10 μM) and Thio T (Sigma) at a final concentration of 10 μM was added, and aggregation was induced by the addition of a freshly prepared heparin sodium salt solution (Santa Cruz) at a final concentration of 44 μg/ml. For non-induced controls, assay buffer was added in place of heparin solution to a final volume of 100 μl per well. Aggregation reactions were carried out at 37° C. with continuous shaking and monitored via ThT fluorescence (excitation, 444 nm; emission, 485 nm; cutoff, 480 nm) in a Spectramax M5 microplate reader (Molecular Devices). Readings were taken every 5 min for a minimum of 24 h.

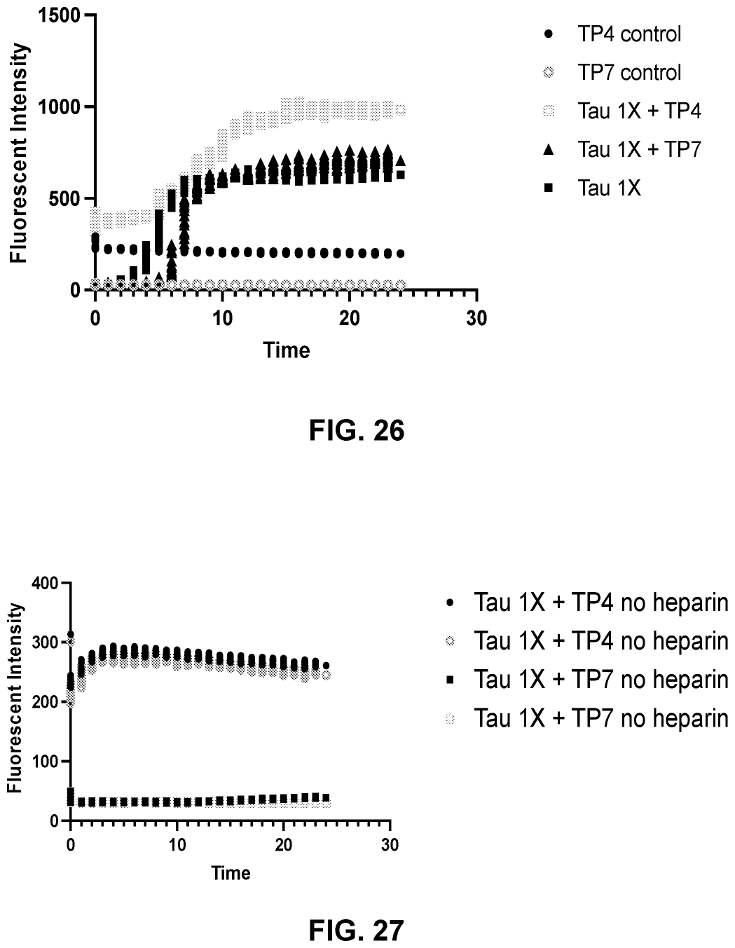

The results depicted in FIG. 26, suggest that mimetic aggregative polymers accelerate or amplify fibril formation of tau.

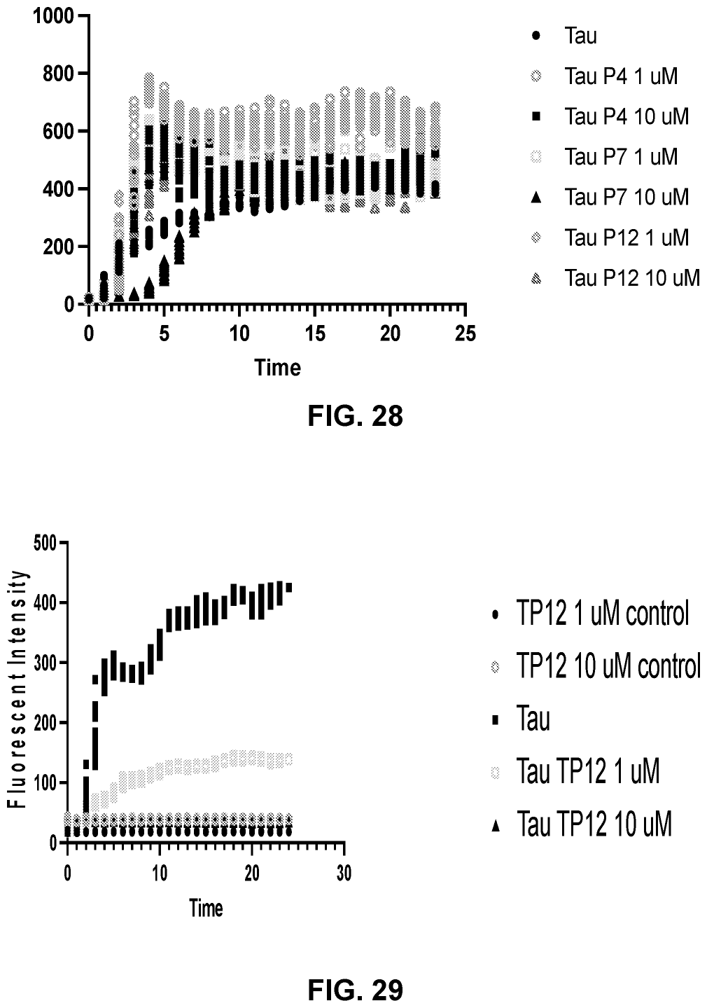

The mimetic polymers were subjected to additional experimentation to determine whether they acted as crowding agents and induced fibrilization of K18 Tau in the absence of heparin. Surprisingly, they did not (FIG. 27), though this is not necessarily relevant to biological systems where crowding is a natural phenomenon. Furthermore, based on the beta sheet forming peptide controls (FIG. 24), the PLPs effect on Tau fibrilization was evaluated at two different concentrations: 1 μM to match the polymer and 10 μM to semi-match the actual concentration of peptide on the polymer. Unfortunately, the amplitude that was “unstable,” and the plotting software GraphPad Prism could not compare if the resulting values shown in FIG. 28 were significant. More replicates are expected to smooth the amplitude and allow for comparison.

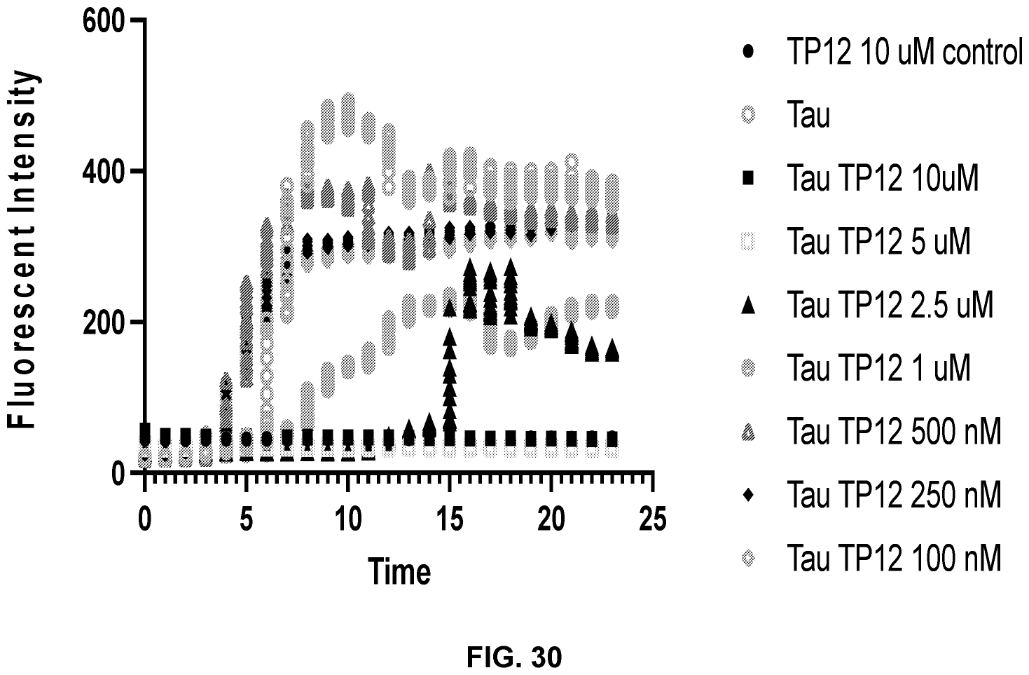

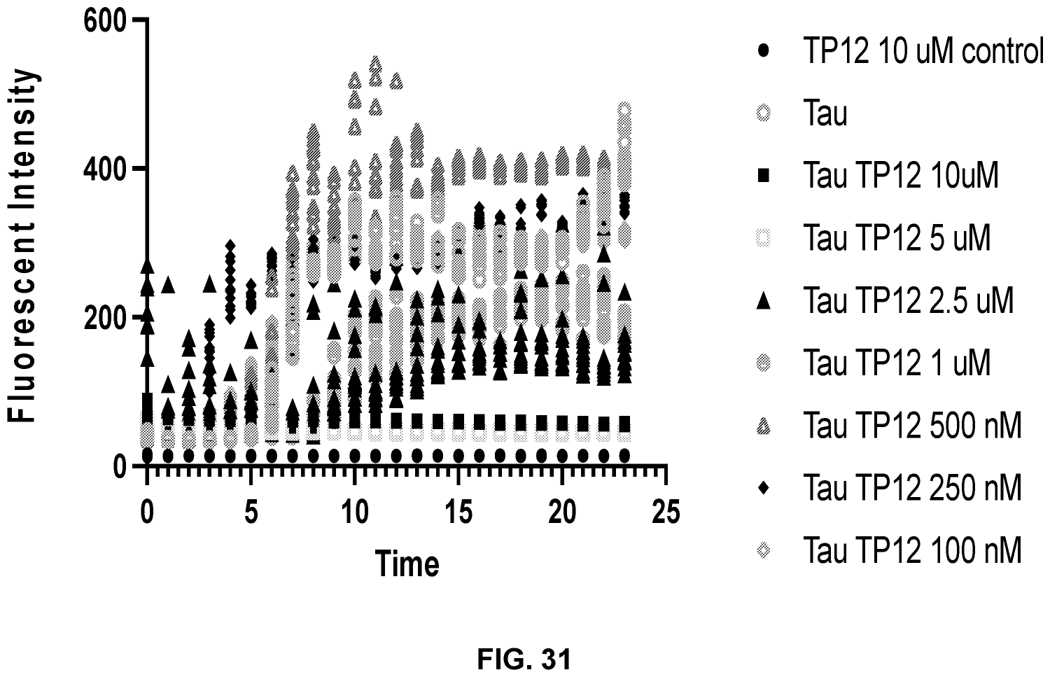

Tau Kinetics Thioflavin T assays were conducted using TP12 (i.e., PLP incorporating SEQ ID: 10—a known inhibitor). Assays were conducted at two concentrations of TP12, 1 μM and 10 μM, and the results (FIG. 29) suggest that the inhibitor polymer significantly prevented fibril formation of K18 Tau. However, recent attempts to perform concentration gradients of TP12 to ascertain the lowest possible dose for inhibition were disrupted by the lag time of the control K18 Tau (FIGS. 30 and 31). Indeed, the lag time of the control K18 Tau was over 5 hours, which is significantly higher than standards of 2.5 hours.

Table C. Summary of results depicted in FIGS. 26, 28, and 29 in the event the graphical representations are difficult to ascertain. Values are rounded to the nearest whole number. Tau 10 hr refers to the average amplitude (fluorescent intensity readout) of an n=6 of the control Tau (meaning no peptide or polymer added, just heparin, thioflavin T, and PBS) trace at 10 hour. The same applies to Tau 15 hr and Tau 20 hr. Avg Tau amplitude refers to the average of the amplitudes at 10, 15, and 20 hr in the previous columns. As Tau is the control for each individual plate, and these readings were done on three separate plates, there are 3 different sets of average amplitudes. Similar to the aforementioned, PLP or Pep 10 hr refers to the average amplitude (n=6 for PLP and n=3 for peptide) of the Tau trace treated with that compound at 10 hours. Avg P amplitude refers to the average of the amplitude at the 3 times, or the average of the 3 previous columns. PLP or Pep/Tau % is the result of dividing the Avg P amplitude by Avg Tau amplitude and multiplying by 100. % amp change refers to the change in amplitude given by subtracting Tau average amplitude from P average amplitude and dividing by Tau average amplitude and multiplying by 100.

TABLE C

| | | Tau | Tau | Tau | Avg Tau | PLP or | PLP or | PLP or | Avg P | PLP or | % amp |
|---|---|---|---|---|---|---|---|---|---|---|---|
| FIG. | PLP/Pep used | 10 hr | 15 hr | 20 hr | Amplitude | Pep 10 hr | Pep 15 hr | Pep 20 hr | Amplitude | Pep/Tau % | change |
| 26 | TP4 1 μM | 578 | 644 | 609 | 611 | 730 | 936 | 961 | 876 | 143 | 44 |
| 26 | TP7 1 μM | 580 | 644 | 609 | 611 | 633 | 715 | 672 | 673 | 110 | 11 |
| 29 | TP12 1 μM | 325 | 410 | 411 | 382 | 116 | 129 | 143 | 129 | 34 | −69 |
| 29 | TP12 10 μM | 325 | 410 | 411 | 382 | 36 | 37 | 36 | 36 | 9 | −91 |
| 28 | P4 1 μM | 242 | 364 | 411 | 339 | 588 | 558 | 657 | 601 | 177 | 46 |
| 28 | P4 10 μM | 242 | 364 | 411 | 339 | 417 | 497 | 395 | 436 | 129 | 6 |
| 28 | P7 1 μM | 242 | 364 | 411 | 339 | 417 | 411 | 545 | 458 | 135 | 11 |
| 28 | P7 10 μM | 242 | 364 | 411 | 339 | 395 | 448 | 438 | 427 | 126 | 4 |
| 28 | P12 1 μM | 242 | 364 | 411 | 339 | 605 | 555 | 693 | 618 | 182 | 50 |
| 28 | P12 10 μM | 242 | 364 | 411 | 339 | 584 | 413 | 424 | 474 | 140 | 15 |

### Example 8

In this example, the toxicity and off-target effect in cell cultures is evaluated.

To measure the biological effect of tau PLP introduction to cellular environments, monitoring assays, including global transcriptional profiling by RT-PCR and RNA-seq, may be used. Each of which allow for monitoring the acute and chronic effects of PLPs on cellular toxicity, stress responses and effects on proteostasis. Understanding the biological impact is important as the proposed therapeutic pathway is not a single protein-protein interaction in a single cell line, but rather a chain of sequences. It will be verified that autophagy is not induced, and that critical cellular processes are not disrupted by cellular process monitoring assays such as via cell viability.

Tau is highly expressed in neurons and has important roles in maintaining neuronal function. Therefore, it is important to titrate the tau PLPs and tau HYDRACs to ensure that neuronal health is not affected as a function of concentration. The selection of tau PLPs and tau HYDRACs that specifically target pathological tau species are expected to reduce toxic effects. Tau-binding PLPs will have different aqueous solubilities and may result in off-target effects. At high local concentrations of PLPs in the cytosol, intrinsic seeding and amplification properties on endogenous MAPT wild type or P301S mutant proteins should be monitored. Depending on the physiochemical kinetics of events, effects on cellular proteostasis may be observed. In the case of accelerated tau aggregation, sequestration of functional cellular proteins such as chaperones and the autophagy and proteasome machinery will be monitored.

### Example 9

In this example, tau-binding PLPs are used to develop a biosensor in order to detect leaky tau before symptom onset.

Fluorophore-tagged PLPs will be used as biosensor that specifically binds free tau collected from blood samples. Various forms of tau will be doped into pooled blood and optimized tau detection conditions. It is believed the tau mimic PLPs with the lowest k_{off} rate, as determined by BLI, may be best suited for this application in capturing tau, as opposed to PLPs that bind less tightly or are inhibitor based. Screening assays involving tau-binding PLPs as biosensors may be used in parallel with brain scans and other techniques to improve early identification of AD.

### Example 10

In this example, the aggregation and protofibril formation of tau in cell culture is regulated.

Tau-directed PLPs that affect amyloid formation or alter phase transitions in cell-free assays, are screened in cells using the human 293T system that conditionally expresses 1N4R tau that forms amyloid upon seeding with brain formed MAPT P301S fibrils. The cell permeability, subcellular localization, and stability is assessed using fluorophore end-tagged polymers. Intracellular tau is visualized by immunostaining with anti-total tau antibodies (DAKO total tau pAb and HT7 mAb). Colocalization image analysis and biochemical pull-down experiments will detect interaction between fluorescent tau PLPs and intracellular tau. The performance of tau PLPs is evaluated to determine whether tau PLPs show higher affinity to MAPT P301S mutant protein than to wild type tau. The appearance of tau inclusions occurs after exposing HEK293 tau P301S cells to Sarkosyl-insoluble tau seeds derived from the brains of TgP301S mice, tau amyloid is monitored using thioflavin T staining and antibody immunostaining for oligomeric and/or aggregated forms of tau (MCi and PG5)—it is expected that these results will mirror each other. Inhibitor PLPs and HYDRACs are expected to reduce aggregation more than tau mimic homopolymer PLPs. Tau-binding PLPs are delivered before, during and after seeding to understand how they affect tau aggregation at the different stages of the tau seeding/amplification process. For example, addition of tau PLPs before seeding is expected to inhibit the kinetics of seed amplification and thus delay tau amyloid formation, or alternatively stimulate seed amplification and amyloid formation. This would lead alternatively to the development of tau PLPs that inhibit or enhance amyloid formation. Likewise, another class of tau PLPs would lead to dissociation of the tau protofibrillar or amyloid state and would be detected upon addition to these respective states and either block further growth or cause their dissolution. The key here is that PLPs are expected to engage with the expected target, tau, with varying avidity. Design rules and mapping interactions are developed, and studies on how PLPs engage tau to either prevent seeding or growth of tau amyloid in the context of complex liquid-liquid phase separated species is performed.

### Example 11

In this example, the ability of PLPs described herein to degrade pre-formed aggregates in cell culture is evaluated.

The overall goal of the tau HYDRAC is the disruption and degradation of tau aggregates before they become neurofibrillary tangles. It is believed that PROTACs generally work by decreasing the proximity of a protein of interest to E3 ligase (via an E3 ligase recruiter, in this case Von Hippel-Lindau protein (VHL)) that facilitates polyubiquitination, thus resulting in the degradation of that protein. The HYDRAC applies the PLP system to this modality, wherein success has previously been shown for tau peptide based PROTACs. The HYDRAC is novel because we will have superior resistance to degradation allowing for lower concentration to be delivered for same therapeutic effect as compared to established peptide PROTACs.

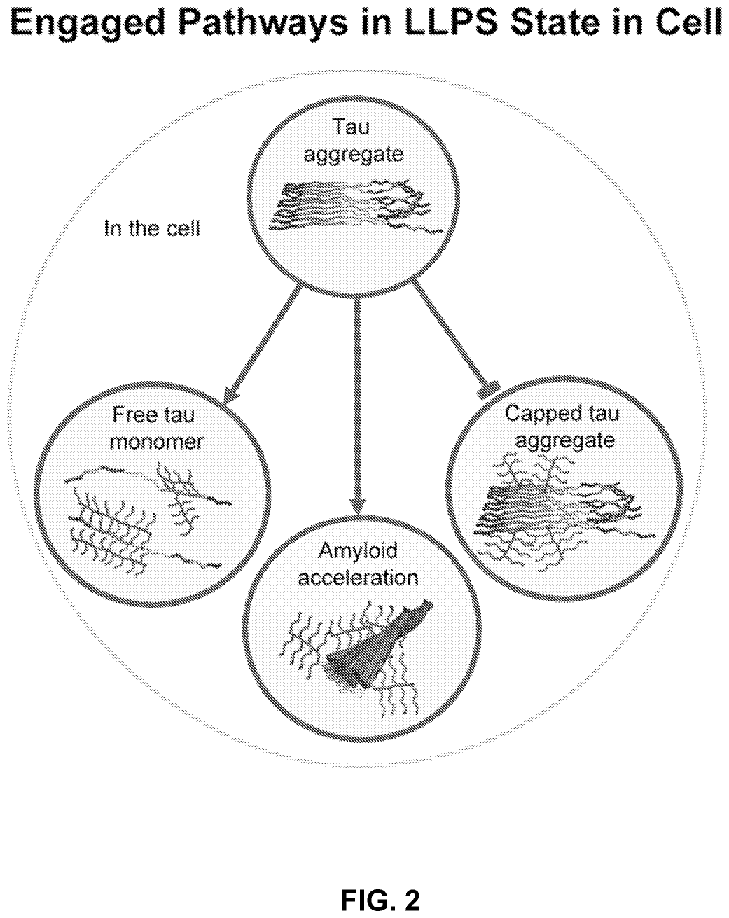

Tau-binding peptides are copolymerized with a peptide motif binding to E3 ubiquitin ligase to create a tau HYDRAC. Tau-targeting HYDRACs are synthesized similar to previously described polymers, wherein a block of targeting peptide sequences will be polymerized first, and upon coming to completion, the addition of the E3 ligase recruiter motif (e.g., SEQ ID: 11 (ALAPYIP) or SEQ ID: 12 (ALAPYIPRR)) will form the second block. The block copolymer will be characterized via both SEC MALS and SDS PAGE, in case it does not run on SEC MALS given the aggregative nature of the targeting motif. As much of the pathogenic tau is collected in the LLPS condensate state (FIG. 2), HYDRACs will be evaluated for their ability to phase separate, likely being heavily influenced on their targeting motif. Lack of LLPS is not necessarily a detriment, as some free monomeric tau and aggregates exist outside these condensates (FIG. 2).

Tau turnover rate is assessed using pulse-chase methods in HEK 293 MAPT WT and P301S cells, following treatment with tau HYDRACs. It is examined whether increased proteasome degradation is specific for tau protein by following Ub G76V-GFP, a proteasome degradation reporter co-expressed in HEK293 cells. The consequence of tau HYDRACs is quantified using the tau seeding/aggregation assay. A positive result is considered to be any substantial change in aggregation rate measured on the plate reader with the expectation that HYDRACs will significantly disrupt and destroy aggregates. It is hypothesized that the HYDRAC offers a multivalent degradation strategy, a proof of concept to be extended to many other desirable protein targets in neurodegenerative disease and beyond.

With the tau-binding and HYDRAC PLPs initially characterized in the human 293T conditional system, candidates (i.e., PLPs having peptide side chains comprising one or more of SEQ ID: 1-10, SEQ ID: 13-16; and one or more of SEQ ID: 11-12, or any other suitable degrader agent) are assessed using patient derived MAPT direct differentiated induced cortical neurons. The induced cortical neurons from tauopathy patient fibroblasts express high levels of the 4R isoform of tau similar to the Alzheimer's brain and distinct from normal brains that express all 6 tau isoforms with 4R/3R ratio of ˜1. The direct induced neurons retain the epigenetic age-related signature which makes the induced neurons an ideal cellular system to study human tauopathies. See e.g., Huh C J et al., Maintenance of age in human neurons generated by microRNA-based neuronal conversion of fibroblasts. Elife 2016; 5:1-14. Total tau isoform expression and 4R/3R tau ratio is determined biochemically following PLPs treatment. Similarly, thioflavin T staining is used to monitor amyloid formation. The PLPs effect on neuronal cell viability and on restoration of proteostasis pathways is evaluated. This allows an assessment of the broad efficiency of tau-targeting PLPs in neurons harboring a variety of disease-associated tau mutations. As a result, PLPs are established as generalizable chemical biology tools in understanding how to inhibit aggregation, and in providing a new polymer-based platform technology for mimicking and enabling new therapeutic modalities in an otherwise unpreventable disease.

Success depends, in part, on the ability of the HYDRACS to permeate tau-expressing cells, whether they be augmented HEK293 or SH-SY5Y cells. The addition of cationic residues (e.g., arginine residues) to peptide sidechains is expected to facilitate this. Arginine-modified PLPs, which primarily enter cells through direct membrane penetration, are expected to allow for efficient cytosolic targeting of tau. However, it is possible that the cell surface interaction of arginine-rich peptides may activate small GTPase Rac, leading to actin organization, lamellipodia formation, and macropinocytic uptake. If this membrane-bound uptake route is taken, tau-binding PLPs could be mislocalized to the endocytic vesicular trafficking pathway, which would affect the efficiency of tau targeting. The entry mechanism of tau-targeting PLPs will be examined and if necessary, additional peptide modifications (e.g., utilizing endosomal escape peptides or acid sensitive functional groups) may be made to increase their vesicular escape.

Upon permeating cells, HYDRACS are expected to localize with tau. The experiments discussed in Examples 6 and 7 will be assessed with fluorophore labeled polymers and visualized via confocal microscopy, specifically looking for the signal of the labeled HYDRAC to be within the confines of the cell membrane and in another experiment, for it to overlap with the tau staining. Localization with tau also indicates whether polymers will induce degradation when tau signal is quantified in cells via antibody staining or Thioflavin T staining.

The HYDRAC mechanism of action will be investigate further by probing this pathway by inhibiting proteasomes (e.g. via MG132) and autophagy (e.g. via bafilomycin A1) to determine if the polyubiquitination guided by the HYDRAC is indeed the main source of degradation of tau. Western blots probed for aggregated tau with specific antibodies will elucidate the levels of tau in cell samples, but also in tissue samples for in vivo studies. Treated and non-treated transgenic mice brains will be collected and lysed so total tau aggregate concentration can be quantified by Western blot and compared to non-treated and treated wild type controls.

### Example 12

In this example, in vivo pharmacokinetics, biodistribution, and disease model efficacy is evaluated.

The PLP platform lends itself to facile labelling with fluorophores, biotin, and rare earth metal complexes for sensitive detection. Previously, such labels have been used to aid in quantifying PLPs in blood and organs for pharmacokinetic (PK) and biodistribution (BD) analysis. Furthermore, PLP elimination half-lives of 152 hours have been observed, with the compound being detectable in the blood and brain one week after a single injection. See e.g., Blum A P, et al. Peptides displayed as high density brush polymers resist proteolysis and retain bioactivity. J Am Chem Soc 2014; 136:15422-37; Blum A P, et al., Activating peptides for cellular uptake via polymerization into high density brushes. Chem Sci 2016; 7:989-94; Thompson M P et al., Labelling polymers and micellar nanoparticles via initiation, propagation and termination with ROMP. Polym Chem 2014; 5:1954-64, each of which is incorporated by reference herein in its entirety, and more specifically to demonstrate the facile labelling of PLPs and detection capabilities, to the extent not inconsistent with the description herein. Biotin tags will be added to the most successful tau PLP and tau HYDRAC to examine PK and BD profile following weekly tail vein (IV) injections in Balb/c mice for an 8 week treatment. Successful tau PLPs and tau HYDRAC are chosen by each PLP's utility in degrading aggregates to rescue cells (HYDRAC) or disrupting/accelerating aggregates to ameliorate tau pathology.

Elimination half-life of the biotin-tagged-tau PLP and tau HYDRAC leads are measured separately following sacrifice and exsanguination and preparation for biotin quantification. It is expected this will provide a quantification via the streptavidin-biotin detection complex in a commercially available kit. Standard curves from whole blood doped with each PLP are made to ensure accurate quantification is obtained from the workup. Additional organs, such as the brain, liver, and kidneys, are collected for maceration and biotin quantification and pathology. 18 timepoints spanning an 8 week treatment period are expected to allow full characterization of the PK/BD profile and confirm that tau PLPs not only reach the brain, but have no toxic effect on the mice. To address scientific rigor, both female and male mice will be assessed in these studies.

For validation following human neuronal cell experiments, the corresponding MAPT P301S tau mutation transgenic mice are treated with lead tau PLP and tau HYDRAC for behavioral and neuropathological efficacy. Animal motor skills and memory are assessed with rotarod and open fielding testing and weekly measures of body weight. Neuropathology is assessed upon sacrifice with western blot and immunohistochemistry detection of tau in the brain. Both female and male mice will be assessed in these studies, which will include 55 male and 55 female mice, distributed across various wild-type and disease groups.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX—YY” (wherein XX and YY refer to claim numbers) is intended to provide a multiple dependent claim in the alternative form, and in some embodiments is interchangeable with the expression “as in any one of claims XX—YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A polymer comprising a first repeating unit comprising a first polymer backbone subunit directly or indirectly covalently linked to a first functional sidechain comprising a peptide, which (i) inhibits aggregation of, (ii) accelerates aggregation of, (iii) binds to, and/or (iv) mimics at least a portion of Tau protein.

2. A polymer comprising a first repeating unit comprising a first polymer backbone subunit directly or indirectly covalently linked to a first functional sidechain comprising a peptide, which (i) inhibits aggregation of, (ii) accelerates aggregation of, (iii) binds to, and/or (iv) mimics at least a portion of microtubulin protein.

3. A polymer characterized by a formula (FX1): 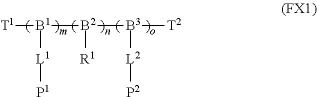
wherein:
each P¹ independently comprises a peptide;
each P² independently comprises a peptide, and each instance of P² is different from each instance of P¹;
at least one P¹ independently, or in combination with other instances of P¹, (i) inhibits aggregation of, (ii) accelerates aggregation of, (iii) binds to, and/or (iv) mimics:
(a) at least a portion of Tau protein, and/or
(b) at least a portion of microtubulin protein;
T¹ and T² are each independently polymer backbone terminating groups that can be the same or different;
B¹, B², and B³ are each independently a polymer backbone subunit;
L¹ and L² are each independently a linking group;
R¹ is independently a substituent;
m is an integer from 2 to 1000;
n is an integer from 0 to 1000;
o is an integer from 0 to 1000;
each connecting line in the formula (FX1) represents a covalent linkage comprising at least one of a single bond, a double bond, one or more atoms, or any combination thereof, optionally wherein the one or more atoms comprise carbon, nitrogen, and/or oxygen atoms;
each instance of B¹, B², B³, L¹, L², R¹, P¹, and P² is the same as or different from any other instance of B¹, B², B³, L¹, L², R¹, P¹, and P², respectively; and
when (i) n is an integer from 1 to 1000, o is an integer from 1 to 1000, at least one instance of P¹ is different from another instance of P¹, and/or at least one instance of P² is different from another instance of P², then (ii) the polymer is a block copolymer or a statistical copolymer.

4. The polymer of claim 3, wherein:
each instance of P¹ independently comprises from 5 to 50 amino acids.

5. The polymer of claim 3 or 4, wherein:
at least one P¹ comprises a sequence having 75% or greater sequence identity of:
SEQ ID NO: 1
(VQIVYK);
SEQ ID NO: 2
(DRVQIVYKRR);
SEQ ID NO: 3
(VQIINK);
SEQ ID NO: 4
(VQIINKRR);
SEQ ID NO: 5
(KVQIINKKLDRR);
SEQ ID NO: 6
(YQQYQDATADEQG);
SEQ ID NO: 7
(YQQYQDATADEQGRRR);
SEQ ID NO: 8
(WMINK);
SEQ ID NO: 9
(WMINKRR);
SEQ ID NO: 10
(VQPINK);
SEQ ID NO: 13
(VQIVYKRR);
SEQ ID NO: 14
(DRWMINKRR);
SEQ ID NO: 15
(VQPINKR);
or
SEQ ID NO: 16
(YQQYQDATADEQGRR).

6. The polymer of any one of claims 3-5, wherein:
at least one P¹ comprises a sequence having 85% or greater sequence identity of:
SEQ ID NO: 1
(VQIVYK);
SEQ ID NO: 2
(DRVQIVYKRR);
SEQ ID NO: 3
(VQIINK);
SEQ ID NO: 4
(VQIINKRR);
SEQ ID NO: 5
(KVQIINKKLDRR);
SEQ ID NO: 6
(YQQYQDATADEQG);
SEQ ID NO: 7
(YQQYQDATADEQGRRR);
SEQ ID NO: 8
(WMINK);
SEQ ID NO: 9
(WMINKRR);
SEQ ID NO: 10
(VQPINK);
SEQ ID NO: 13
(VQIVYKRR);
SEQ ID NO: 14
(DRWMINKRR);
SEQ ID NO: 15
(VQPINKR);
or
SEQ ID NO: 16
(YQQYQDATADEQGRR).

7. The polymer of any one of claims 3-6, wherein:
at least one P¹ comprises:
SEQ ID NO: 1
(VQIVYK);
SEQ ID NO: 2
(DRVQIVYKRR);
SEQ ID NO: 3
(VQIINK);
SEQ ID NO: 4
(VQIINKRR);
SEQ ID NO: 5
(KVQIINKKLDRR);
SEQ ID NO: 6
(YQQYQDATADEQG);
SEQ ID NO: 7
(YQQYQDATADEQGRRR);
SEQ ID NO: 8
(WMINK);
SEQ ID NO: 9
(WMINKRR);
SEQ ID NO: 10
(VQPINK);
SEQ ID NO: 13
(VQIVYKRR);
SEQ ID NO: 14
(DRWMINKRR);
SEQ ID NO: 15
(VQPINKR);
or
SEQ ID NO: 16
(YQQYQDATADEQGRR).

8. The polymer of any one of claims 3-7, wherein:
at least a portion of each instance of P¹ independently comprises:
SEQ ID NO: 1
(VQIVYK);
SEQ ID NO: 2
(DRVQIVYKRR);
SEQ ID NO: 3
(VQIINK);
SEQ ID NO: 4
(VQIINKRR);
SEQ ID NO: 5
(KVQIINKKLDRR);
SEQ ID NO: 6
(YQQYQDATADEQG);
SEQ ID NO: 7
(YQQYQDATADEQGRRR);
SEQ ID NO: 8
(WMINK);
SEQ ID NO: 9
(WMINKRR);
SEQ ID NO: 10
(VQPINK);
SEQ ID NO: 13
(VQIVYKRR);
SEQ ID NO: 14
(DRWMINKRR);
SEQ ID NO: 15
(VQPINKR);
or
SEQ ID NO: 16
(YQQYQDATADEQGRR).
or
any combination thereof.

9. The polymer of any one of claims 3-8, wherein:
at least 75% of all instances of P¹ on a number basis independently comprise:
SEQ ID NO: 1
(VQIVYK);
SEQ ID NO: 2
(DRVQIVYKRR);
SEQ ID NO: 3
(VQIINK);
SEQ ID NO: 4
(VQIINKRR);
SEQ ID NO: 5
(KVQIINKKLDRR);
SEQ ID NO: 6
(YQQYQDATADEQG);
SEQ ID NO: 7
(YQQYQDATADEQGRRR);
SEQ ID NO: 8
(WMINK);
SEQ ID NO: 9
(WMINKRR);
SEQ ID NO: 10
(VQPINK);
SEQ ID NO: 13
(VQIVYKRR);
SEQ ID NO: 14
(DRWMINKRR);
SEQ ID NO: 15
(VQPINKR);
or
SEQ ID NO: 16
(YQQYQDATADEQGRR);
or
any combination thereof.

10. The polymer of any one of claims 3-9, wherein:
at least one P¹ comprises a sequence having 75% or greater sequence identity of:
SEQ ID NO: 2
(DRVQIVYKRR);
SEQ ID NO: 4
(VQIINKRR);
SEQ ID NO: 10
(VQPINK);
or
SEQ ID NO: 15
(VQPINKR).

11. The polymer of any one of claims 3-10, wherein:
at least one P¹ comprises a sequence having 85% or greater sequence identity of:
SEQ ID NO: 2
(DRVQIVYKRR);
SEQ ID NO: 4
(VQIINKRR);
SEQ ID NO: 10
(VQPINK);
or
SEQ ID NO: 15
(VQPINKR).

12. The polymer of any one of claims 3-11, wherein:
at least one P¹ comprises:
SEQ ID NO: 2
(DRVQIVYKRR);
SEQ ID NO: 4
(VQIINKRR);
SEQ ID NO: 10
(VQPINK);
or
SEQ ID NO: 15
(VQPINKR).

13. The polymer of any one of claims 3-12, wherein:
at least a portion of each instance of P¹ independently comprises:
SEQ ID NO: 2
(DRVQIVYKRR);
SEQ ID NO: 4
(VQIINKRR);
SEQ ID NO: 10
(VQPINK);
or
SEQ ID NO: 15
(VQPINKR);
or
any combination thereof.

14. The polymer of any one of claims 3-13, wherein:
at least 75% of all instances of P¹ on a number basis comprise:
SEQ ID NO: 2
(DRVQIVYKRR);
SEQ ID NO: 4
(VQIINKRR);
SEQ ID NO: 10
(VQPINK);
or
SEQ ID NO: 15
(VQPINKR);
or
any combination thereof

15. The polymer of any one of claims 3-14, wherein:
at least one P¹ comprises:
SEQ ID NO: 1
(VQIVYK)
or
SEQ ID NO: 2
(DRVQIVYKRR);
and
at least one other P¹ comprises:
SEQ ID NO: 8
(WMINK)
or
SEQ ID NO: 9
(WMINKRR).

16. The polymer of claim 15, wherein the polymer is characterized by proportionate amounts of the at least one P¹ and the at least one other P¹.

17. The polymer of claim 15, wherein the polymer is characterized by a peptide ratio of the at least one P¹: the at least one other P¹ of 1:14.

18. The polymer of claim 15, wherein the polymer is characterized by a peptide ratio of the at least one P¹: the at least one other P¹ of 7:8.

19. The polymer of claim 15, wherein the polymer is characterized by a peptide ratio of the at least one P¹: the at least one other P¹ of 14:1.

20. The polymer of any one of claims 3-19, wherein:
at least one P¹ comprises:
SEQ ID NO: 4
(VQIINKRR)
or
SEQ ID NO: 3
(VQIINK);
and
at least one other P¹ comprises:
SEQ ID NO: 8
(WMINK)
or
SEQ ID NO: 9
(WMINKRR).

21. The polymer of claim 20, wherein the polymer is characterized by proportionate amounts of the at least one P¹ and the at least one other P¹.

22. The polymer of claim 20, wherein the polymer is characterized by a peptide ratio of the at least one P¹: the at least one other P¹ of 1:14.

23. The polymer of claim 20, wherein the polymer is characterized by a peptide ratio of the at least one P¹: the at least one other P¹ of 7:8.

24. The polymer of claim 20, wherein the polymer is characterized by a peptide ratio of the at least one P¹: the at least one other P¹ of 14:1.

25. The polymer of any one of claims 3-24, wherein:
at least one P¹ comprises:
SEQ ID NO: 10
(VQPINK)
or
SEQ ID NO: 15
(VQPINKR);
and
at least one other P¹ comprises:
SEQ ID NO: 3
(VQIINK),
SEQ ID NO: 4
(VQIINKRR),
SEQ ID NO: 5
(KVQIINKKLDRR),
SEQ ID NO: 1
(VQIVYK),
or
SEQ ID NO: 2
(DRVQIVYKRR).

26. The polymer of claim 25, wherein the polymer is characterized by proportionate amounts of the at least one P¹ and the at least one other P¹.

27. The polymer of claim 25, wherein the polymer is characterized by a peptide ratio of the at least one P¹: the at least one other P¹ of 1:14.

28. The polymer of claim 25, wherein the polymer is characterized by a peptide ratio of the at least one P¹: the at least one other P¹ of 8:7.

29. The polymer of claim 25, wherein the polymer is characterized by a peptide ratio of the at least one P¹: the at least one other P¹ of 14:1.

30. The polymer of any one of claims 3-29, wherein:
each instance of P², if present, independently comprises 5 to 50 amino acids.

31. The polymer of any one of claims 3-30, wherein:
o is an integer from 1 to 1000, and
at least one P² comprises a sequence that is capable of triggering an organism's ubiquitination cellular machinery, optionally wherein the amino acid sequence binds to E3 ubiquitin ligase.

32. The polymer of claim 31, wherein:
the sequence that is capable of triggering an organism's ubiquitination cellular machinery (i) has at least 75% or at least 90% amino acid composition similarity to, (ii) has at least 60% or at least 80% sequence identity to, or (iii) comprises:
SEQ ID NO: 11
(ALAPYIP)
or
SEQ ID NO: 12
(ALAPYIPRR).

33. The polymer of claim 31, wherein:
at least one P¹ comprises SEQ ID NO: 6 (YQQYQDATADEQG), SEQ ID NO: 7 (YQQYQDATADEQGRRR), SEQ ID NO: 16 (YQQYQDATADEQGRR), SEQ ID NO: 3 (VQIINK), SEQ ID NO: 4 (VQIINKRR), SEQ ID NO: 5 (KVQIINKKLDRR), SEQ ID NO: 1 (VQIVYK), or SEQ ID NO: 2 (DRVQIVYKRR); and
at least one P² comprises SEQ ID NO: 11 (ALAPYIP) or SEQ ID NO: 12 (ALAPYIPRR).

34. The polymer of any one of claims 3-33, wherein o is an integer from 1 to 1000 and the polymer is characterized by a peptide ratio of P¹:P² of 14:1.

35. The polymer of any one of claims 3-34, wherein o is an integer from 1 to 1000 and the polymer is characterized by a peptide ratio of P¹:P² of 13:2.

36. The polymer of any one of claims 3-35, wherein o is an integer from 1 to 1000 and the polymer is characterized by a peptide ratio of P¹:P² of 5:1.

37. The polymer of any one of claims 3-36, wherein o is an integer from 1 to 1000 and the polymer is characterized by a peptide ratio of P¹:P² of 3:1.

38. The polymer of any one of claims 3-37, wherein:
the tau protein and/or microtubulin protein are in a form of oligomers, protofibrils, amyloid fibrils, cross-β sheet amyloid species, or any combination thereof.

39. The polymer of any one of claims 3-38, wherein:
at least one P¹ independently, or in combination with other instances of P¹, (i) inhibits aggregation of, (ii) accelerates aggregation of, (iii) binds to, and/or (iv) mimics (a) at least a portion of tau protein when the tau protein is in a liquid-liquid phase separated (LLPS) state.

40. The polymer of claim 39, wherein:
the polymer disrupts and/or prevents further growth of the LLPS state of the tau protein.

41. The polymer of any one of claims 3-40, wherein:
the polymer is capable of being in an LLPS state under physiological conditions.

42. The polymer of any one of claims 3-41, wherein:
(a) the at least a portion of tau protein comprises an aggregative region of the tau protein,
(b) the at least a portion of microtubulin protein comprises an aggregative region of the microtubulin protein; or
(c) a combination thereof.

43. The polymer of any one of claims 3-42, wherein:
at least one P¹ and/or at least one P² is characterized by a net positive charge.

44. The polymer of any one of claims 3-43, wherein:
at least one P¹ and/or at least one P² is characterized by a net positive charge of 1 to 5.

45. The polymer of any one of claims 3-44, wherein:
at least 75% of all instances of P¹ on a number basis are characterized by a net positive charge.

46. The polymer of any one of claims 3-45, wherein:
at least 75% of all instances of P² on a number basis are characterized by a net positive charge.

47. The polymer of any one of claims 3-46, wherein:
at least one P¹ and/or at least one P² comprises or further comprises at least one arginine.

48. The polymer of any one of claims 3-47, wherein:
at least one P¹ and/or at least one P² comprises or further comprises at least one aspartic acid.

49. The polymer of any one of claims 3-48, wherein:
at least one P¹ and/or at least one P² comprises or further comprises at least one proline.

50. The polymer of any one of claims 1-49, wherein:
the polymer is metaphilic.

51. The polymer of any one of claims 1-50, wherein the polymer is characterized by an average degree of polymerization of 2 to 100.

52. The polymer of any one of claims 1-51, wherein the polymer is characterized by an average degree of polymerization of 2 to 50.

53. The polymer of any one of claims 1-52, wherein the polymer is characterized by an average degree of polymerization of 2 to 30.

54. The polymer of any one of claims 1-53, wherein the polymer is characterized by an average degree of polymerization of 5 to 30.

55. The polymer of any one of claims 1-54, wherein the polymer is characterized by a number average molecular weight of 1 kDa to 50 kDa.

56. The polymer of any one of claims 1-55, wherein the polymer is characterized by a number average molecular weight of 1 kDa to 30 kDa.

57. The polymer of any one of claims 1-56, wherein the polymer is characterized by a brush density of at least 75%.

58. The polymer of any one of claims 1-57, wherein the polymer is characterized by a brush density of at least 80%.

59. The polymer of any one of claims 3-58, wherein the polymer has at least one of the following properties:
(a) P¹ comprises 5-100 amino acids;
(b) m is an integer from 2 to 100;
(c) n is an integer from 0 to 100;
(d) o is an integer from 0 to 100;
(d) m is an integer from 2 to 100, n is 0, p is 0, and at least one instance of P¹ is different from another instance of P¹;
(e) degree polymerization (m+n+o) is an integer from 2 to 200, or 2 to 50;
(f) molecular weight of from 1 kDa to 1,000 kDa;
(g) the polymer has a P¹ peptide density of at least 50%, as defined by equation m/(m+n+o)×100;
(h) a combination thereof,
(i) any combination thereof.

60. The polymer of any one of claims 3-59, wherein m is an integer from 2 to 100, and the polymer binds multivalently via at least two P's to the at least a portion of tau protein and/or the at least a portion of microtubulin protein.

61. The polymer of any one of claims 3-60, wherein at least one of B¹, B², or B³ is, or all three are, a polymerized monomer comprising an unsaturated monomer.

62. The polymer of claim 61, wherein the unsaturated monomer comprises an ethylenically unsaturated monomer, a norbornene monomer, or a norbornene dicarboxyimide.

63. The polymer of any one of claims 1-62, wherein the polymer is prepared by a living polymerization method optionally selected from ring-opening metathesis polymerization (ROMP), reversible addition-fragmentation chain transfer polymerization (RAFT), or atom transfer radical polymerization (ATRP).

64. The polymer of any one of claims 3-63, wherein each instance of a substructure (Sla): 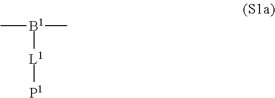
in formula (FX1) independently comprises a substructure (S1b) or a substructure (S1c): 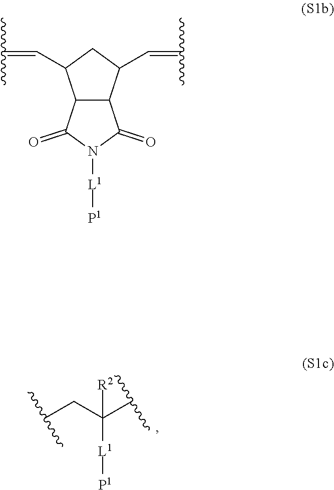
wherein R² is H or C₁-C₃ alkyl.

65. The polymer of any one of claims 3-64, wherein each instance of a substructure (S2a): 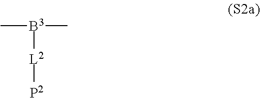
in formula (FX1) independently comprises a substructure (S2b) or a substructure (S2c): 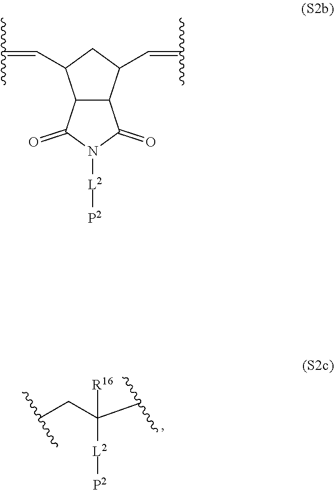
wherein R¹⁶ is H or C₁-C₃ alkyl.

66. The polymer of any one of claims 3-65, wherein each instance of L and L², if present, independently is a single bond, —O—, —(CH₂CH₂O)ₓ—, C₁-C₁₀ alkyl, C₁-C₁₀ acyl, C₂-C₁₀ alkenyl, C₃-C₁₀ aryl, C₁-C₁₀ alkoxyl, or any combination thereof, wherein x is an integer from 1 to 20, and wherein each L¹ and L², if present, is configured with one or more suitable functional groups to covalently attach B¹ with P¹ and B³ with P².

67. The polymer of any one of claims 3-66, wherein each of R¹, T¹, and T² independently is hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₁₀ alkynyl, C₅-C₃₀ alkylaryl, —CO₂R³, —CONR⁴R⁵, —COR⁶, —SOR⁷, —OSR⁸, —SO₂R⁹, —OR¹⁰, —SR¹¹, —NR²R¹³, —NR¹⁴COR¹, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, a metal complex, a fluorophore-containing moiety, or a contrast agent-containing moiety, wherein each of R³—R¹⁵ independently is H, C₅-C₁₀ aryl, or C₁-C₁₀ alkyl.

68. The polymer of any one of claims 3-67, wherein at least one P¹, P², R¹, T¹, or T² comprises, or further comprises, a fluorophore-containing moiety or a contrast agent-containing moiety.

69. The polymer of any one of claims 3-68, wherein at least one P¹, P², R¹, T¹, or T² comprises, or further comprises, rhodamine, fluorescein, Cy5.5, gadoteric acid, or a combination thereof.

70. A composition comprising the polymer of any one of claims 1-69 and a pharmaceutically acceptable carrier.

71. A medicament, for use in preventing, disrupting, accelerating, or detecting tau and/or microtubulin protein aggregation, comprising a therapeutically effective amount of a composition having the polymer of any one of claims 1-69.

72. A medicament, for use in preventing, treating, or detecting a tauopathy-related disease or condition in a subject, comprising a therapeutically effective amount of a composition having the polymer of any one of claims 1-69.

73. A method for preventing, disrupting, accelerating, or detecting tau and/or microtubulin protein aggregation, the method comprising:
contacting an oligomer, protofibril, amyloid fibril, and/or cross-0 sheet amyloid species of tau and/or microtubulin with a therapeutically effective amount of the polymer of any one of claims 1-69 or the composition of claim 70.

74. The method of claim 73, wherein the oligomer, protofibril, amyloid fibril, and/or cross-β sheet amyloid species of tau and/or microtubulin is in a patient or in a fluid derived from a subject.

75. The method of claim 73, wherein at least one R¹ is a fluorophore-containing moiety or a contrast agent-containing moiety, and the method further comprises an imaging step after the contacting step.

76. A method for preventing, treating, or detecting a tauopathy-related disease or condition in a subject, the method comprising:
administering to the subject a therapeutically effective amount of the polymer of any one of claims 0-69 or the composition of claim 70.

77. The method of claim 76, wherein the tauopathy-related disease or condition comprises a neurodegenerative disease optionally selected from or associated with Alzheimer's disease (AD), primary age-related tauopathy, chronic traumatic encephalopathy, traumatic brain injury, progressive supranuclear palsy, corticobasal degeneration, dementia, frontotemporal dementia, argyrophilic grain disease, frontotemporal dementia and parkinsonism linked to chromosome 17, Parkinson's disease, parkinsonism, postencephalitic parkinsonism, amyotrophic lateral sclerosis (ALS), Huntington's disease, vacuolar tauopathy, lytico-bodig disease, ganglioglioma, gangliocytoma, meningioangiomatosis, subacute sclerosing panencephalitis, lead encephalopathy, tuberous sclerosis, pantothenate kinase-associated neurodegeneration, lipofuscinosis, Pick's disease, Pick's complex, or any combination thereof.

78. The method of claim 76, wherein the polymer is administered to the subject's brain, spinal cord, cerebrospinal fluid, or any combination thereof.

79. The method of any one of claims 76-78, wherein at least one R¹ is a fluorophore-containing moiety or a contrast agent-containing moiety, and the method further comprises an imaging step after the administering step.

80. Use of a composition for preventing, disrupting, accelerating, or detecting tau and/or microtubulin protein aggregation, wherein the composition comprises the polymer of any one of claims 1-69.

81. Use of a composition for preventing, treating, or detecting a tauopathy-related disease or condition in a subject, wherein the composition comprises the polymer of any one of claims 1-69.

82. A method of making the polymer of any one of claims 0-69, the method comprising:
synthesizing the at least one P¹ peptide;
capping the at least one P¹ peptide at a terminal end with a polymerizable monomer that, once polymerized, becomes polymer backbone subunit B¹, thereby forming a polymerizable P¹ monomer;
polymerizing the polymerizable P¹ monomer.

83. The method of claim 82, wherein:
the synthesizing step comprises solid-phase synthesis using protected amino acids;
the polymerizable monomer comprises an ethylenically unsaturated monomer optionally comprising norbornene or a (meth)acrylate; and
the polymerizing step comprises ROMP, RAFT, or ATRP.

84. A method of making a medicament for use in preventing, disrupting, accelerating, or detecting tau and/or microtubulin protein aggregation, the method comprising combining:
a therapeutically effective amount of a composition having the polymer of any one of claims 1-69, and
an optional carrier.

85. A method of making a medicament for use in preventing, treating, or detecting a tauopathy-related disease or condition in a subject, the method comprising combining:
a therapeutically effective amount of a composition having the polymer of any one of claims 1-69, and
an optional carrier.


SEQUENCE LISTING (16 sequences)

SEQ ID NO: 1 - protein, 6 aa, synthetic construct
VQIVYK

SEQ ID NO: 2 - protein, 10 aa, synthetic construct
DRVQIVYKRR

SEQ ID NO: 3 - protein, 6 aa, synthetic construct
VQIINK

SEQ ID NO: 4 - protein, 8 aa, synthetic construct
VQIINKRR

SEQ ID NO: 5 - protein, 12 aa, synthetic construct
KVQIINKKLDRR

SEQ ID NO: 6 - protein, 13 aa, synthetic construct
YQQYQDATADEQG

SEQ ID NO: 7 - protein, 16 aa, synthetic construct
YQQYQDATADEQGRRR

SEQ ID NO: 8 - protein, 5 aa, synthetic construct
WMINK

SEQ ID NO: 9 - protein, 7 aa, synthetic construct
WMINKRR

SEQ ID NO: 10 - protein, 6 aa, synthetic construct
VQPINK

SEQ ID NO: 11 - protein, 7 aa, synthetic construct
ALAPYIP

SEQ ID NO: 12 - protein, 9 aa, synthetic construct
ALAPYIPRR

SEQ ID NO: 13 - protein, 8 aa, synthetic construct
VQIVYKRR

SEQ ID NO: 14 - protein, 9 aa, synthetic construct
DRWMINKRR

SEQ ID NO: 15 - protein, 7 aa, synthetic construct
VQPINKR

SEQ ID NO: 16 - protein, 15 aa, synthetic construct
YQQYQDATADEQGRR